(19) United States
(12) Patent Application Publication
(10) Pub. No.: US 2009/0258025 A1
(43) Pub. Date: Oct. 15, 2009

(54) TREATMENTS AND DIAGNOSTICS FOR CANCER, INFLAMMATORY DISORDERS AND AUTOIMMUNE DISORDERS

(75) Inventors: Paul J. Godowski, Hillsborough, CA (US); Joachim Lehmann, Basel (CH); Ganesh A. Kolumam, San Mateo, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/171,576

(22) Filed: Jul. 11, 2008

Related U.S. Application Data
(60) Provisional application No. 61/003,499, filed on Nov. 16, 2007.
(60) Provisional application No. 60/959,726, filed on Jul. 13, 2007.

(51) Int. Cl. A61K 39/395; C12Q 1/02; G01N 33/567; C12N 5/06

(52) U.S. Cl. 424/158.1; 435/29; 435/7.21; 435/373

(57) ABSTRACT

Methods for the treatment of cancer with therapies targeting tumor-associated macrophage activities are provided. Methods for the treatment of cancer, inflammatory and autoimmune disorders with therapies using tumor-associated macrophages and adipose tissue macrophages are also provided.

### RELATED APPLICATIONS

This application is a nonprovisional application claiming priority under 35 USC 119(e) to provisional application No. 60/959,726, filed Jul. 13, 2007, and to provisional application No. 61/003,499, filed Nov. 16, 2007, the contents of which are incorporated herein by reference.

### FIELD OF THE INVENTION

The invention relates to the field of tumor growth. The invention relates to activities and characteristics of tumor-associated macrophages, and uses of such for the diagnosis and treatment of cancer and tumor growth. The invention also relates to the field of immunology and uses of tumor-associated macrophage and adipose tissue macrophage activities and characteristics for treating autoimmune and inflammatory disorders.

### BACKGROUND

Malignant tumors (cancers) are a leading cause of death in the United States, after heart disease. Cancer is characterized by the increase in the number of abnormal, or neoplastic, cells derived from a normal tissue which proliferate to form a tumor mass, the invasion of adjacent tissues by these neoplastic tumor cells, and the generation of malignant cells which eventually spread via the blood or lymphatic system to regional lymph nodes and to distant sites via a process called metastasis. In a cancerous state, a cell proliferates under conditions in which normal cells would not grow. Cancer manifests itself in a wide variety of forms, characterized by different degrees of invasiveness and aggressiveness.

Human tumors are comprised of both malignant and non-malignant cells. This latter category includes stromal fibroblasts, endothelial cells and leukocytes. Tumor-associated macrophages (“TAM”) are a prominent component of the leukocytic infiltrate in most solid tumors. In some instances, TAM can comprise up to 50% of the total tumor mass (Kelly et al. 1988; O'Sullivan and Lewis 1994; Leek et al. 1994; Bingle et al. 2002). High levels of macrophage infiltrates in breast carcinomas and other human tumors have been correlated with poor prognosis. Analysis of murine models of mammary cancer supports the view that TAM promote growth and metastasis of tumors. For example, inhibition of TAM differentiation in a genetic model of mammary cancer reduces the rate of tumor progression and dramatically reduces metastasis formation in the lung (Lin et al. 2001).

One proposed mechanism by which TAM may contribute to the growth of human breast cancer is by the production of angiogenic factors such as vascular endothelial growth factor; high levels of TAM have been correlated with increased vascular density within breast tumors (Leek et al. 1996; Lin et al. 2006). Myeloid lineage hematopoietic cells, including TAMs, have been shown to stimulate angiogenesis either directly by secreting angiogenic factors or indirectly by producing extracellular matrix-degrading proteases, which in turn release sequestered angiogenic factors (reviewed in Lewis, C. E. & Pollard, J. W. Distinct role of macrophages in different tumor microenvironments. Cancer Research 66:605-612 (2006); and, Naldini, A. & Carraro, F. Role of inflammatory mediators in angiogenesis. Curr Drug Targets Inflamm Allergy 4:3-8 (2005)). Among the myeloid cell lineages, CD11b⁺Gr1⁺ progenitor cells isolated from the spleens of tumor-bearing mice promoted angiogenesis when co-injected with tumor cells (see, e.g., Yang, L. et al. Expansion of myeloid immune suppressor Gr⁺CD11b⁺ cells in tumor-bearing host directly promotes tumor angiogenesis. Cancer Cell 6:409-21 (2004)) and tumor-infiltrating macrophage numbers correlated with poor prognosis in some human tumors (reviewed in Balkwill et al. in Balkwill, F., Charles, K. A. & Mantovani, A. Smoldering and polarized inflammation in the initiation and promotion of malignant disease. Cancer Cell 7:211-7 (2005)). However, in another study, macrophages inhibited growth of experimental tumors in mice, suggesting their potential as anticancer therapy. See, e.g., Kohchi, C. et al. Utilization of macrophages in anticancer therapy: the macrophage network theory. Anticancer Res 24:3311-20 (2004).

It has been suggested that, in addition to promoting angiogenesis, TAM may also contribute to tumor growth by promoting inflammation, matrix remodeling, tumor cell invasion, intravasation and seeding at distant sites (Lewis et al. 2000; Lewis and Poloard 2006; Pollard 2004; Hiratsuka et al. 2002; Lin et al. 2001; Sica et al. 2006).

TAM are derived from circulating monocytes, which are recruited to the malignant tissue by tumor-derived chemokines. Monocytes are distinguished by their versatility and plasticity and, depending upon their specific microenvironment, can differentiate into macrophages with a variety of activation stages. These activation ranges are operationally defined across two distinct polarization states, M1 and M2. While these states have been defined in vitro, it is thought that tissue macrophages exist along a continuum of M1 and M2. In an environment dominated by pro-inflammatory stimuli and type I cytokines, monocytes differentiate into M1 macrophages that express high levels of pro-inflammatory cytokines, promote Th1 immune responses and mediate resistance to intracellular parasites. Conversely, an environment in which Type II cytokines (i.e. IL-4 and IL-13) predominate promotes the generation of M2 or “trophic” macrophages. M2 macrophages are immuno-regulatory and promote tissue repair and remodeling.

It has been proposed that TAM as “trophic” M2 macrophages have an indirect role in inducing tolerance by secreting certain cytokines such as IL-6, CSF-1, IL-10 and TGFβ which are thought to inhibit the maturation of dendritic cells (“DC”) in tumors (Mantovani et al. 2002; Pollard 2004). DC are professional antigen presenting cells with the ability to induce and regulate immune responses, and usually undergo maturation after antigen capture in tissue. They upregulate MHC II expression and co-stimulatory molecules and migrate to the draining lymph nodes, where they can induce a potent T cell response. DC that capture antigen under non-inflammatory conditions (i.e. in tumor tissue) may not fully mature and thus be impaired in antigen presentation. These immature or semi-mature DC express low levels of co-stimulatory proteins and potentially generate regulatory T lymphocytes that potentiate tolerogenic responses (Steinman et al. 2003).

Several subsets of regulatory T cells have been defined based on site of origin, expression of phenotypic markers, and suppressive mechanism. A particularly well-characterized subset is the naturally occurring thymus-derived CD4⁺ CD25⁺ T regulatory cells. Such cells express high levels of FoxP3 and GITR and mediate immune suppression through a cell contact-dependent mechanism. A second CD4⁺ subset, Trl cells, is induced in peripheral tissue and mediates immune suppression in a contact-independent manner via the secretion of IL-10 and/or TGFβ. Increasing evidence supports the importance of regulatory T cells in inhibiting the immune response to tumors. Several reports document the existence of elevated numbers of regulatory FoxP3⁺ CD4⁺ T cells (Leong et al. 2006; Liyanage et al. 2002) and IL-10⁺ CD4⁺ Trl cells (Marshall et al. 2004; Seo et al. 2001) in solid tumors. Furthermore, elevated levels of FoxP3⁺ CD4⁺ T cells in human breast cancer samples correlate with reduced overall survival rates (Curiel et al. 2004; Bates et al. 2006).

Despite the presence of TAM in tumor infiltrate and their potential to produce angiogenic factors, their role in tumor growth and development remains unclear. There is a need to discover and understand the biological functions of TAM, and the factors that they produce. The present invention addresses these and other needs, as will be apparent upon review of the following disclosure.

### SUMMARY OF THE INVENTION

In one embodiment, the invention provides a method of identifying inflammation-related tissue macrophages (IRTM) within a sample, comprising contacting the sample with an IRTM binding agent and determining the presence of one or more cells to which the IRTM binding agent is associated. In one aspect, the sample is a tissue sample. In another aspect, the sample is human. In another aspect, the IRTM binding agent is an antibody or antigen-binding fragment thereof. In another aspect, the IRTM are tumor associated macrophages (TAM). In another aspect, the IRTM are adipose tissue macrophages (ATM).

In another embodiment, the invention provides a method of identifying inflammation-related tissue macrophages (IRTM) within a sample, comprising contacting the sample with at least one first agent that specifically recognizes a cell surface marker specific for macrophages and at least one second agent that specifically recognizes a cell surface marker specific for dendritic cells and determining the presence of cells recognized by both the at least one first agent and the at least one second agent. In one aspect, the at least one first agent and/or the at least one second agent specifically bind to the cell surface marker specific for macrophages or the cell surface marker specific for dendritic cells. In another such aspect, the at least one first agent and/or the at least one second agent are antibodies or antigen-binding fragments thereof. In another aspect, the at least one first agent and the at least one second agent are the same molecule. In another such aspect, the molecule is selected from the group consisting of a bispecific antibody, a trispecific antibody, an antibody with greater than three different specificities, and an antigen-binding fragment of any of the recited antibodies. In another aspect, the cell surface marker specific for macrophages is F4/80. In another aspect, the cell surface marker specific for dendritic cells is CD11c. In another aspect, determining the presence of cells recognized by both the at least one first agent and the at least one second agent comprises at least one method selected from the group consisting of immunohistochemistry, fluorescence-activated cell sorting, magnetic cell sorting, affinity chromatography, fluorescent in situ hybridization, and immunomicroscopy. In another aspect, the cell sample is a tumor sample. In another aspect, the IRTM is a TAM. In another aspect, the IRTM is an ATM.

In another embodiment, the invention provides a method of isolating TAM from a mixture of cells, comprising (a) contacting the cell sample with at least one first agent that specifically recognizes a cell surface marker specific for macrophages and at least one second agent that specifically recognizes a cell surface marker specific for dendritic cells, and (b) isolating cells recognized by both the at least one first agent and the at least one second agent. In one aspect, the at least one first agent and/or the at least one second agent specifically bind to the cell surface marker specific for macrophages or the cell surface marker specific for dendritic cells. In another aspect, the at least one first agent and/or the at least one second agent are antibodies or antigen-binding fragments thereof. In another aspect, the at least one first agent and the at least one second agent are the same molecule. In another such aspect, the molecule is selected from the group consisting of a bispecific antibody, a trispecific antibody, an antibody with greater than three different specificities, and an antigen-binding fragment of any of the recited antibodies. In another aspect, the cell surface marker specific for macrophages is F4/80. In another aspect, the cell surface marker specific for dendritic cells is CD11c. In another aspect, the isolating step comprises at least one of fluorescence-activated cell sorting, affinity chromatography, and magnetic cell sorting.

In another embodiment, the invention provides a method of diagnosing a proliferative disorder in a subject, comprising determining the presence and/or activity of TAM in the subject. In one aspect, the determining step comprises contacting a sample of cells from the subject with at least one first agent that specifically recognizes a cell surface marker specific for macrophages and at least one second agent that specifically recognizes a cell surface marker specific for dendritic cells, and identifying cells recognized by both the at least one first agent and the at least one second agent. In another aspect, the proliferative disorder is breast cancer. In another aspect, the at least one first agent and/or the at least one second agent specifically bind to the cell surface marker specific for macrophages or the cell surface marker specific for dendritic cells. In one such aspect, the at least one first agent and/or the at least one second agent are antibodies or antigen-binding fragments thereof. In another such aspect, the at least one first agent and the at least one second agent are the same molecule. In another such aspect, the molecule is selected from the group consisting of a bispecific antibody, a trispecific antibody, an antibody with greater than three different specificities, and an antigen-binding fragment of any of the recited antibodies. In another such aspect, the cell surface marker specific for macrophages is F4/80. In another such aspect, the cell surface marker specific for dendritic cells is CD11c. In another such aspect, the identifying step comprises at least one method selected from the group consisting of immunohistochemistry, fluorescence-activated cell sorting, magnetic cell sorting, affinity chromatography, fluorescence in situ hybridization, and immunomicroscopy.

In another embodiment, the invention provides method of staging a tumor in a subject, comprising determining the presence and/or activity of TAM in the subject. In one aspect, the determining step comprises contacting a sample of cells from the subject with at least one first agent that specifically recognizes a cell surface marker specific for macrophages and at least one second agent that specifically recognizes a cell surface marker specific for dendritic cells, and identifying cells recognized by both the at least one first agent and the at least one second agent. In another aspect, the tumor is a breast cancer tumor. In another aspect, the at least one first agent and/or the at least one second agent specifically bind to the cell surface marker specific for macrophages or the cell surface marker specific for dendritic cells. In another such aspect, the at least one first agent and/or the at least one second agent are antibodies or antigen-binding fragments thereof. In another such aspect, the at least one first agent and the at least one second agent are the same molecule. In another such aspect, the molecule is selected from the group consisting of a bispecific antibody, a trispecific antibody, an antibody with greater than three different specificities, and an antigen-binding fragment of any of the recited antibodies. In another such aspect, the cell surface marker specific for macrophages is F4/80. In another such aspect, the cell surface marker specific for dendritic cells is CD11c. In another aspect, the identifying step comprises at least one method selected from the group consisting of immunohistochemistry, fluorescence-activated cell sorting, magnetic cell sorting, affinity chromatography, fluorescence in situ hybridization, and immunomicroscopy.

In another embodiment, the invention provides a method of treating a tumor in a subject, comprising modulating TAM viability or activity. In one aspect, modulating TAM viability or activity comprises selective removal of TAM from a tumor cell population or tumor sample. In one such aspect, the selective removal of TAM comprises (a) contacting the population or sample with a TAM binding agent and (b) selectively removing those cells specifically bound to the TAM binding agent from the population or sample. In another such aspect, the TAM binding agent comprises at least one antibody and the selective removal step is selected from antibody-mediated clearance, protein A chromatography, affinity chromatography, fluorescence activated cell sorting, and magnetic cell sorting. In another aspect, modulating TAM viability or activity comprises selectively killing TAM within a tumor cell population or tumor sample. In one such aspect, selectively killing TAM comprises (a) contacting the population or sample with a TAM binding agent and (b) selectively killing those cells specifically bound to the TAM binding agent from the population or sample. In another such aspect, the TAM binding agent comprises at least one antibody and the selective killing step is complement-mediated cytotoxicity. In another such aspect, the TAM binding agent comprises at least one antibody and the selective killing step is mediated by a cytotoxic molecule conjugated to the antibody. In another aspect, modulating TAM viability or activity comprises inhibiting TAM activity within a tumor cell population or tumor sample. In one such aspect, inhibiting TAM activity comprises inhibiting secretion or activity of one or more TAM-secreted cytokine or TAM-secreted chemokine in the population or sample. In another such aspect, the TAM-secreted cytokine is TGFβ. In another such aspect, inhibiting secretion or activity of one or more TAM-secreted cytokine or TAM-secreted chemokine comprises administering a TAM-secreted cytokine/chemokine binding agent. In another such aspect, the TAM-secreted cytokine/chemokine binding agent is selected from an antibody or antigen-binding fragment, a receptor specific for the cytokine or chemokine, or a small molecule inhibitory to the activity of the cytokine/chemokine. In another aspect, inhibiting secretion or activity of one or more TAM-secreted cytokine or TAM-secreted chemokine comprises administering an antagonist of a TAM-secreted cytokine/chemokine. In another aspect, the subject is a human subject. In another aspect, the method further comprises co-administration or sequential administration of one or more additional therapeutic agents selected from the group consisting of a chemotherapeutic agent, a cytokine, a chemokine, an anti-angiogenic agent, an immunosuppressive agent, a cytotoxic agent, an anti-inflammatory agent, and a growth inhibitory agent.

In another embodiment, the invention provides a method of treating an autoimmune disorder in a subject, comprising modulating TAM viability or activity. In one aspect, modulating TAM viability or activity comprises stimulating TAM activity. In one such aspect, stimulating TAM activity comprises administering one or more compounds selected from the group consisting of a TAM agonist and an agonist of TAM-secreted cytokine/chemokine. In another such aspect, stimulating TAM activity results in induction of at least one of FoxP3+CD4⁺ T regulatory cells, IL-10⁺CD4⁺ T regulatory cells, and inflammatory TH₁₇ cells. In another aspect, the subject is a human subject. In another aspect, the method further comprises co-administration or sequential administration of one or more additional therapeutic agents selected from the group consisting of a cytokine, a chemokine, a cytotoxic agent, and an immunosuppressive agent.

In another embodiment, the invention provides a method of inhibiting tolerogenesis in a subject, comprising modulating TAM viability or activity. In one aspect, modulating TAM viability or activity comprises selective removal of TAM. In one such aspect, the selective removal of TAM comprises (a) administering a TAM binding agent and (b) selectively removing those cells specifically bound to the TAM binding agent. In another such aspect, the TAM binding agent comprises at least one antibody and the selective removal step is antibody-mediated clearance. In another aspect, modulating TAM viability or activity comprises selectively killing TAM. In one such aspect, selectively killing TAM comprises (a) administering a TAM binding agent and (b) selectively killing those cells specifically bound to the TAM binding agent. In another such aspect, the TAM binding agent comprises at least one antibody and the selective killing step is complement-mediated cytotoxicity. In another such aspect, the TAM binding agent comprises at least one antibody or antigen-binding fragment and the selective killing step is mediated by a cytotoxic molecule conjugated to the antibody or antigen-binding fragment. In another aspect, modulating TAM viability or activity comprises inhibiting TAM activity. In one such aspect, inhibiting TAM activity comprises inhibiting secretion or activity of one or more TAM-secreted cytokine or TAM-secreted chemokine. In one such aspect, the TAM-secreted cytokine is TGFβ. In another such aspect, inhibiting secretion or activity of one or more TAM-secreted cytokine or TAM-secreted chemokine comprises administering a TAM-secreted cytokine/chemokine binding agent. In one such aspect, the TAM-secreted cytokine/chemokine binding agent is selected from an antibody or antigen-binding fragment, a receptor specific for the cytokine or chemokine, or a small molecule inhibitory to the activity of the cytokine/chemokine. In another aspect, inhibiting secretion or activity of one or more TAM-secreted cytokine or TAM-secreted chemokine comprises administering an antagonist of a TAM-secreted cytokine/chemokine. In another aspect, the subject is a human subject. In another aspect, the method further comprises co-administration or sequential administration of one or more additional therapeutic agents selected from the group consisting of a cytokine, a chemokine, a cytotoxic agent, an anti-inflammatory, and an immunosuppressive agent.

In another embodiment, the invention provides a method for selectively inducing growth and/or proliferation of FoxP3⁺ CD4⁺ T regulatory cells, IL-10⁺CD4⁺ Trl cells, or inflammatory TH₁₇ cells, comprising administering IRTM to naïve T cells or otherwise exposing naïve T cells to IRTM under conditions appropriate for normal cell growth. In one aspect, the IRTM is a TAM. In another aspect, the IRTM is an ATM. In one aspect, the method further comprises administering one or more compounds selected from a TAM and/or ATM agonist and an agonist of TAM and/or ATM-secreted cytokine/chemokines. In another aspect, the method further comprises isolating the induced FoxP3⁺ CD4⁺ T regulatory cells, IL-10⁺CD4⁺ Trl cells, or inflammatory TH₁₇ cells.

In another embodiment, the invention provides a method of treating an inflammatory disorder in a subject, comprising modulating IRTM viability or activity. In one aspect, modulating IRTM viability or activity comprises stimulating IRTM activity. In another aspect, stimulating IRTM activity comprises administering one or more compounds selected from the group consisting of an IRTM agonist and an agonist of an IRTM-secreted cytokine/chemokine. In another aspect, stimulating IRTM activity results in induction of at least one of FoxP3⁺ CD4⁺ T regulatory cells, IL-10⁺CD4⁺ Trl cells, or inflammatory TH₁₇ cells. In another aspect, the subject is a human subject. In another aspect, the method further comprises co-administration or sequential administration of one or more additional therapeutic agents selected from the group consisting of a cytokine, a chemokine, a cytotoxic agent, an anti-inflammatory, and an immunosuppressive agent. In another aspect, modulating IRTM viability or activity comprises selective removal of IRTM. In another aspect, the selective removal of IRTM comprises (a) administering an IRTM binding agent and (b) selectively removing those cells specifically bound to the IRTM binding agent. In another aspect, the IRTM binding agent comprises at least one antibody and the selective removal step is antibody-mediated clearance. In another aspect, modulating IRTM viability or activity comprises selectively killing IRTM. In another aspect, selectively killing IRTM comprises (a) administering an IRTM binding agent and (b) selectively killing those cells specifically bound to the IRTM binding agent. In another aspect, the IRTM binding agent comprises at least one antibody and the selective killing step is complement-mediated cytotoxicity. In another aspect, the IRTM binding agent comprises at least one antibody or antigen-binding fragment and the selective killing step is mediated by a cytotoxic molecule conjugated to the antibody or antigen-binding fragment. In another aspect, modulating IRTM viability or activity comprises inhibiting IRTM activity. In another aspect, inhibiting IRTM activity comprises inhibiting secretion or activity of one or more IRTM-secreted cytokine or IRTM-secreted chemokine. IN another aspect, the IRTM-secreted cytokine is TGFβ. In another aspect, inhibiting secretion or activity of one or more IRTM-secreted cytokine or IRTM-secreted chemokine comprises administering an IRTM-secreted cytokine/chemokine binding agent. In another aspect, the IRTM-secreted cytokine/chemokine binding agent is selected from an antibody or antigen-binding fragment, a receptor specific for the cytokine or chemokine, or a small molecule inhibitory to the activity of the cytokine/chemokine. In another aspect, inhibiting secretion or activity of one or more IRTM-secreted cytokine or IRTM-secreted chemokine comprises administering an antagonist of an IRTM-secreted cytokine/chemokine. In another aspect, the IRTM is selected from TAM and ATM. In another aspect, the subject is a human subject. In another aspect, the method further comprises co-administration or sequential administration of one or more additional therapeutic agents selected from the group consisting of a cytokine, a chemokine, a cytotoxic agent, an anti-inflammatory, and an immunosuppressive agent.

In another embodiment, the invention provides a method for selectively inducing growth and/or proliferation of FoxP3⁺ CD4⁺ T regulatory cells, IL-10⁺CD4⁺ Trl cells, and/or inflammatory TH₁₇ cells comprising exposing naïve T cells to TAM and/or ATM under conditions appropriate for normal cell growth. In one aspect, the method further comprises administering one or more compounds selected from a TAM agonist, an ATM agonist, an agonist of TAM-secreted cytokine/chemokines, and an agonist of ATM-secreted cytokine/chemokines. In another aspect, the method further comprises isolating the induced FoxP3⁺ CD4⁺ T regulatory cells, IL-10⁺CD4⁺ Trl cells, or inflammatory TH₁₇ cells.

### BRIEF DESCRIPTION OF THE FIGURES

FIGS. 1A-1H depict the results of immunohistochemical analyses of tumor samples, as described in Example 1. FIG. 1A depicts anti-CD45 antibody staining of tumor tissue showing a prominent leukocyte infiltrate. FIG. 1B depicts anti-F4/80 antibody staining of tumor tissue to identify macrophages. FIG. 1C depicts anti-CD3 antibody staining of tumor tissue to identify T cells. FIG. 1D is a graph showing the relative proportions of immune cells in the CD45⁺ lymphoid tumor infiltrate. FIG. 1E is a graph showing the relative proportions of immune cells in the NK1.1⁻ DX5⁻ CD11b⁺ tumor myeloid infiltrate. FIG. 1F depicts tumor samples stained with both anti-F4/80 and anti-CD31 antibodies to show the localization of TAM in the tumor tissue relative to endothelial cells. FIG. 1G depicts tumor samples stained with both anti-Ly-6G and anti-CD31 antibodies to show the localization of neutrophils in the tumor tissue relative to endothelial cells. FIG. 1H depicts tumor samples stained with both anti-Ly-6C and anti-CD31 antibodies to show the localization of inflammatory monocytes (Mo^{IF}) in the tumor tissue relative to endothelial cells. The data in FIGS. 1F-1H represents 2 to 3 repetitions and 6-7 individual tumors.

FIGS. 2A-2C depict graphically the results of FACS analyses assessing the leukocyte composition of MMTV-PyMT tumors. In all three figures, FVB control samples are shown in white and PyMT^{tg} samples are shown in stripes. FIG. 2A shows a 2.3-fold increase in the total number of peripheral blood mononuclear cells (PBMC) in PyMT-induced tumors as compared to tumor free control FVB mouse samples. FIG. 2B shows an increase in CD11b⁺ myeloid PBMC (Nk1.1⁻DX5⁻) cells in tumor-bearing mice as compared to tumor-free control FVB mice. FIG. 2C shows an increase in the neutrophil:monocyte ratio in PyMT tumor mice as compared to control mice. The notation “*” indicates that the data was significant with p≦0.05; the notation “**” indicated that the data was significant with p≦0.01.

FIGS. 3A-3E depict the results of experiments described in Example 2A showing that TAM have features of both macrophages and dendritic cells. FIG. 3A depicts the results of a gene expression analysis showing the CD11c mRNA expression levels of bmDC (white bar), peritoneal macrophages (black bar) and PyMT^{tg}-derived TAM (striped bar) (left-most panel). FIG. 3A also depicts the results of FACS analyses showing that TAM express high levels of CD11c, and F4/80, whereas bmDC or peritoneal macrophages express either CD11c or F4/80 (rightmost three panels). FIGS. 3B-3C depict the results of immunohistochemical analyses showing that frozen sections of PyMT^{tg}-derived tumors (FIG. 3B) or isolated F4/80⁺ TAM cultured for 60 hours in vitro (FIG. 3C) express the dendritic cell marker CD11c. FIG. 3D depicts a gene expression analysis of CD207 mRNA expression levels of bmDC (white bars), peritoneal macrophages (striped bar) and PyMT^{tg}-derived TAM (spotted bar) (leftmost panel). FIG. 3D also depicts the results of FACS analyses showing that CD11b⁺F4/80⁺CD11c⁺ TAM express langerin (CD207). The data in FIG. 3D is representative of four experiments. FIG. 3E depicts the results of real-time PCR experiments showing the expression levels of TGFβ RI, Runx3, and IRF-8 in bmDC (white bars), peritoneal macrophages (striped bars) and PyMT^{tg}-derived TAM (spotted bars).

FIGS. 4A-4C depict the results of experiments described in Example 2A showing the immune cell composition of tumor draining axillary and brachial lymph nodes of PyMT^{tg} mice as compared to tumor-free FVB mice. FIG. 4A shows graphically that elevated number of CD11b⁺ cells were identified in the lymph nodes from the tumor-containing mice. FIG. 4B shows FACS results indicating that increased numbers of CD11b⁺ cells coexpressing CD11c and F4/80 were identified in the lymph nodes from the tumor-containing mice. FIG. 4C depicts photomicrograms showing that the morphology of TAM is closer to that of bmDC than it is to macrophages.

FIGS. 5A-5C depict the results of microarray analyses of expressed genes in TAM, peritoneal macrophages, and bmDC, as described in Example 2A. FIG. 5A shows a heatmap image of expressed genes in those three cell populations, where white coloration indicates a minimum level of relative expression and black coloration indicates a maximal level of relative expression, with grey indicating relative expression of 1. FIG. 5B shows a statistical PC analysis of the gene expression profiling of TAM, peritoneal macrophages, and bmDC, showing close relations between TAM and peritoneal macrophages (left panel) and a graphic visualization of degrees of importance of the individual principal components analyzed (right panel). FIG. 5C depicts the results of a statistical PC analysis of the gene expression profiling of PyMT^{tg}-derived TAM and Her2^{tg}-derived TAM, demonstrating the unique gene profile of TAM as compared to other tissue macrophages (peritoneal macrophages and splenic macrophages and Kupffer cells) (left panel) and a graphic visualization of degrees of importance of the individual principal components analyzed (right panel). The data in FIGS. 5B and 5C average 3-5 mRNA preparations from individually isolated populations.

FIGS. 6A-6C show several FACS analyses assessing TAM surface expression of MHC II and costimulatory molecules CD80, CD83 and CD86, as described in Example 2B. FIG. 6A depicts FACS results for TAM expression of MHC II, CD80, CD83, and CD86. FIG. 6B depicts FACS results for peritoneal macrophage expression of MHC II, CD80, CD83, and CD86. FIG. 6C depicts FACS results for bmDC expression of MHC II, CD80, CD83, and CD86.

FIGS. 7A-7B depict the results of microarray analyses of chemokine and cytokine expression in TAM versus peritoneal macrophages, as described in Example 3. FIG. 7A shows the expression levels of chemokines CCL2, CXCL10, CCL3, CCL5, and KC in both cell populations. FIG. 7B shows the expression levels of cytokines IL-1α, IL-1β, TNFα, IL-10, and IL-6 in both cell populations. FIG. 7C depicts the results of real-time RT-PCR experiments showing the expression levels of TGFβ₁ in bmDC (white bar), peritoneal macrophages (spotted bar), PyMT^{tg}-derived TAM (lightly striped bar) and tumor cells (boldly striped bar). Data shown are the average of 3-5 independent experiments.

FIGS. 8A-8C depict the results of FACS analyses assessing TAM effects on naïve T cells, as described in Example 4. FIG. 8A shows graphically the relative amounts of the cytokines IL-10, IL-4, IL-2 and IL-17 produced by naïve T cell cultures stimulated with TAM, peritoneal macrophages, or bmDC. FIG. 8B depicts FACS results showing that TAM-activated T cells produce IL-10 and IL-17. FIG. 8C graphically depicts the results of immunostaining experiments showing that cytokine secretion from TAM-stimulated CD4⁺ T cells was dependent on TGFβ secretion by TAM.

FIGS. 9A-9D depict the results of FACS analyses described in Example 4 to investigate FoxP3⁺ regulatory T cell induction by TAM. FIG. 9A depicts FACS analyses showing the differences in FoxP3⁺ T cell induction in cultures treated with either TAM or bmDC. FIG. 9B depicts the results of FACS analyses showing the effect of inclusion of TGFβRII on TAM-induction of FoxP3⁺ T cells. FIG. 9C depicts the results of FACS analyses assessing the presence of GITR on the TAM-induced FoxP3⁺ T cells as a marker for regulatory T cells. FIG. 9D depicts the results of FACS analyses assessing the expression of CD103 on TAM-induced FoxP3⁺ T cells as a marker for peripherally-induced regulatory T cells.

FIGS. 10A-10D depict the results of experiments to confirm that TAM induced FoxP3⁺ T cells as opposed to stimulating clonal expansion of preexisting FoxP3⁺ T cells, as described in Example 4. FIG. 10A depicts the results of a FACS analysis assessing the amount of FoxP3⁺ T cells in the preparation of naïve CD4⁺ T cells used herein. FIG. 10B shows the results of experiments analyzing the stimulatory capacity of bmDC, TAM, and peritoneal macrophages on CFSE-labeled naïve CD4⁺ T cells. FIG. 10C depicts the results of FACS analyses assessing the pool of FoxP3⁺ T cells in whole splenocytes (FIG. 10C). FIG. 10D depicts the results of FACS analyses assessing the pool of FoxP3⁺ T cells upon isolation from purified CD103⁺CD25⁺CD69⁺ T cells (FIG. 10D) and retreatment with TAM.

FIGS. 11A-11C depict the results of experiments assessing the in vivo incidence of IL-10⁺ and FoxP3⁺ regulatory T cells in PyMT mice (FIG. 11A) versus control mice (FIG. 11B), as described in Example 5. FIG. 11C shows graphically the relative amounts and/or absolute numbers of FoxP3⁺ CD4⁺ T cells found in tumor draining lymph nodes (leftmost two panels), spleens (center and center right panels), and tumors (rightmost panel) from PyMT mice (striped bars and black circles) versus control mice (white bars and circles).

FIGS. 12A-G depict the results of experiments performed on adipose tissue macrophages (ATM), as described in Example 6. FIG. 12A depicts the results of FACS analysis showing CD11b⁺ cell content. Data are representative of 20 individual fat tissue isolations. FIG. 12B depicts the results of FACS analyses showing the expression of CD11c, MHC II and CD86 in F4/80⁺ ATM. Data are representative of 14 (CD11c) or 5 (MHC II or CD86) individual fat tissue isolations from several different experiments. FIG. 12C depicts the results of FACS analyses showing expression of CD14, ICOS L and TIM3 expression in single cell ATM suspensions derived from epididymal fat of male C57BI/6 mice kept under HFD. Data are representative of 5 individual fat tissue isolations from several different experiments. FIG. 12D depicts the results of FACS analyses showing expression of CD14, ICOS L and TIM3 expression in TAM. Data are representative of 5 individual fat tissue isolations from several different experiments. FIG. 12E depicts the cytokine profile of ATM derived from epididymal fat tissue (striped bars), C57BI/6 wildtype peritoneal macrophages (white bars) or lean tissue macrophages (spotted bars). Data are representative of 6 mice from two experiments. FIG. 12F depicts the results of real-time RT-PCR analyses of the expression levels of TGFβ₁ and TGFβRI in TAM (white bars) and ATM (striped bars). Data are representative of 8 TAM and 3 ATM individual RNA probes from 1-3 experiments or 4 individually isolated pools of macrophages. FIG. 12G shows the morphology of freshly isolated ATM, TAM, and peritoneal macrophages stained with H&E.

FIGS. 13A-J show the results of experiments testing the ability of fat tissue, lymph node tissue, and purified ATM to induce FoxP3⁺ regulatory T cells, as described in Example 7. FIG. 13A depicts the results of FACS analyses for FoxP3 expression in CD4⁺ T cells activated with ATM (left panel), lean fat macrophages (LTM) (center panel) or peritoneal macrophages (right panel). Data shown represent two individual mice in a single experiment. FIG. 13B depicts the results of FACS analyses for TGF-β influence on FoxP3 induction by TAM in T cell cultures supplemented with recombinant TGFβRII-Fc. Data shown represent two individual mice in a single experiment. FIGS. 13C and 13D show the results of FACS analyses assessing the relative amount of FoxP3⁺ T regulatory cells in epididymal fat (FIG. 13C) or splenic tissue (FIG. 13D) in CD4⁺ T cells from male Db/Db mice and age-matched C57BI/6 mice. Data shown represent four individual mice in a single experiment. FIG. 13E depicts the results of experiments assessing cytokine production by ATM-activated T cells. White bars correspond to T cells treated with peritoneal macrophages and black bars correspond to T cells treated with ATM. Data shown represent two experiments using two mice each. FIG. 13F depicts the results of FACS analyses assessing the presence of Trl and TH17 cells in T cell populations activated by TAM. Data shown represent two experiments using two mice each. FIGS. 13G-H depict the results of experiments assessing the population of CD4⁺ T cells from tumor draining lymph nodes in C57BI/6 mice fed a high fat diet (FIG. 13G) or wildtype C57BI/6 mice (FIG. 13H) restimulated with PMA/ionomycin, showing the existence of pronounced populations of IL-10⁺ Trl and TH₁₇ T cells in obese mice. FIGS. 13I and 13J show bar graphs depicting the results of experiments assessing the percentage of FoxP3⁺ CD4⁺ T cells in fat tissue (FIG. 13I) or draining lymph node tissue (FIG. 13J) of age-matched control FVB mice (white circles) or male HFD obese C57BI/6 mice (black circles). ** indicates that the experiment has a p≦0.01.

FIGS. 14A-F depict gene expression profiles in selected immune cell and tumor cell populations. FIGS. 14A-C and E depict heatmap profiles of differential expression of cytokines (FIG. 14A), cytokine receptors (FIG. 14B), chemokines (FIG. 14C) and chemokine receptors (FIG. 14E) in tumor cells, PyMT^{tg}-derived TAM, peritoneal macrophages from wild-type FVB mice, and bmDC. FIG. 14D shows the results of experiments to confirm the differential expression of CCL2, CCL3, CCL5 and CXCL10 in peritoneal macrophages (white bars) or PyMT^{tg}-derived TAM (striped bars). The ** indicates a p<0.01. FIG. 14F depicts the results of real-time RT-PCR analysis of CCR6 gene expression in bmDC (white bar), peritoneal macrophages (striped bar) and PyMT^{tg}-derived TAM (spotted bar). In each figure, the data shows gene profiling from 3-5 independent samples or the average of 3-5 independent experiments. In FIGS. 14A-C and E, the lowest expression levels are shown in dark grey, and the highest levels of expression are indicated by light grey; white squares indicate that the data for that particular analysis was not available.

FIGS. 15A-B depict heatmap profiles of differential expression of M1 (FIG. 15A) and M2 (FIG. 15B) marker gene mRNAs from tumor cells, PyMT^{tg}-derived TAM, peritoneal macrophages from wild-type FVB mice, and bmDC. The data shows gene profiling results from 3-5 independent samples. The lowest expression levels are shown in dark grey and the highest levels of expression are indicated by light grey; white squares indicate that the data for that particular analysis was not available.

FIGS. 16A-B depict the results of experiments showing the cytokine and chemokine profiles of naïve T cells activated by certain immune cell populations. FIG. 16A shows TNFα, IL-5, IL-13, and CCL3 expression in naïve T cells stimulated by bmDC (white bars), peritoneal macrophages from FVB mice (striped bars), or TAM (spotted bars). FIG. 16B shows TNFα, IL-5, and IL-13 expression in naïve T cells stimulated by peritoneal macrophages from C57BI/6 mice (white bars) or ATM (striped bars).

FIG. 17 depicts the results of a statistical PC analysis of the gene expression profiling of certain immune cell populations. The left panel shows a graph demonstrating that ATM, CD11c⁻ ATM, and CD11c⁺ ATM have similar gene expression profiles, but possess distinct gene expression profiles from PyMT^{tg} TAM, Her2^{tg} TAM, and peritoneal macrophages (“PF”).

FIG. 18 depicts the cytokine/chemokine profiles of CD11c⁻ ATM (white bars) or CD11c⁺ ATM (striped bars), as described in Example 8. * indicates that the experiment has a p≦0.05; ** indicates that the experiment has a p≦0.01.

FIGS. 19A and 19B depict the cytokine/chemokine profiles of T cells activated by CD11c⁻ ATM (white bars) or CD11c ATM (striped bars), as described in Example 8.

FIG. 20A shows graphs demonstrating that CD11c⁻ ATM have significantly higher mRNA levels encoding CD209a, CD209b, and CD209c (white bars) as compared to CD11c⁺ ATM (striped bars), as described in Example 9. FIG. 20B depicts FACS analyses of CD209b/SIGN-R1 and CD11c on ATM derived from epididymal fat tissue of either non-obese male C57BL/6 mice (8 weeks old) or obese male C57BL/6 mice (24 weeks old, 20 weeks on HFD), as described in Example 9. The data represent an average of three arrays from individually isolated populations of 4-6 independent ATM isolations. * indicates p≦0.05; ** indicates p≦0.01.

### DETAILED DESCRIPTION

### Definitions

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “inflammation-related tissue macrophages” or “IRTM” when used herein refers to a class of immune cells derived from monocytes that are associated with inflammation and one or more disease states. Examples of IRTM include, but are not limited to, tumor-associated macrophages and adipose tissue macrophages. In certain embodiments, IRTM may also include, but not be limited to, alveolar macrophages and macrophages found in the central nervous system in experimental autoimmune encephalomyelitis (EAE).

The term “IRTM binding protein” when used herein refers to a molecule that specifically binds to an IRTM. IRTM binding proteins include, but are not limited to, antibodies or antigen-binding fragments thereof, proteins, peptides, glycoproteins, glycopeptides, glycolipids, polysaccharides, oligosaccharides, bioorganic molecules, peptidomimetics, pharmacological agents and their metabolites, fusion proteins, and receptor molecules that bind to IRTM. Such binding may be, e.g., to a protein at the IRTM cell surface or to some other IRTM cell surface molecule.

The term “tumor-associated macrophage” or “TAM” when used herein refers to a cell derived from a monocyte that can be found in the immune infiltrate associated with a tumor. As shown herein, TAM express both certain macrophage cell surface markers and certain dendritic cell surface markers.

The term “TAM binding protein” when used herein refers to a molecule that specifically binds to TAM. TAM binding proteins include, but are not limited to, antibodies or antigen-binding fragments thereof, proteins, peptides, glycoproteins, glycopeptides, glycolipids, polysaccharides, oligosaccharides, bioorganic molecules, peptidomimetics, pharmacological agents and their metabolites, fusion proteins, and receptor molecules that bind to TAM. Such binding may be, e.g., to a protein at the TAM cell surface or to some other TAM cell surface molecule.

The term “adipose tissue macrophage” or “ATM” when used herein refers to a cell derived from a monocyte that can be found in the immune infiltrate associated with adipose tissue in obese subjects. As shown herein, ATM express both certain macrophage cell surface markers and certain dendritic cell surface markers.

The term “ATM binding protein” when used herein refers to a molecule that specifically binds to ATM. ATM binding proteins include, but are not limited to, antibodies or antigen-binding fragments thereof, proteins, peptides, glycoproteins, glycopeptides, glycolipids, polysaccharides, oligosaccharides, bioorganic molecules, peptidomimetics, pharmacological agents and their metabolites, fusion proteins, and receptor molecules that bind to ATM. Such binding may be, e.g., to a protein at the ATM cell surface or to some other ATM cell surface molecule.

The abbreviations “Mf” and “MΦ” when used herein refer to macrophages. The abbreviations “pMf” and “pMΦ” refer to peritoneal macrophages.

The term “antagonist” when used herein refers to a molecule capable of neutralizing, blocking, inhibiting, abrogating, reducing or interfering with the activities of a protein of the invention including its binding to one or more receptors in the case of a ligand or binding to one or more ligands in case of a receptor. Antagonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Antagonists also include small molecule inhibitors of a protein of the invention, and fusion proteins, receptor molecules and derivatives which bind specifically to protein thereby sequestering its binding to its target, antagonist variants of the protein, antisense molecules directed to a protein of the invention, RNA aptamers, and ribozymes against a protein of the invention.

A “blocking” antibody or an “antagonist” antibody is one which inhibits or reduces biological activity of the antigen it binds. Certain blocking antibodies or antagonist antibodies substantially or completely inhibit the biological activity of the antigen.

The term “IRTM antagonist” when used herein refers to a molecule which binds to an IRTM and inhibits or substantially reduces a biological activity of an IRTM. Non-limiting examples of IRTM antagonists include antibodies, proteins, peptides, glycoproteins, glycopeptides, glycolipids, polysaccharides, oligosaccharides, nucleic acids, bioorganic molecules, peptidomimetics, small molecules, pharmacological agents and their metabolites, transcriptional and translation control sequences, and the like. In one embodiment of the invention, the IRTM antagonist is an antibody, especially an anti-IRTM cell surface marker antibody which binds human IRTM.

The term “TAM antagonist” when used herein refers to a molecule which binds to TAM and inhibits or substantially reduces a biological activity of TAM. Non-limiting examples of TAM antagonists include antibodies, proteins, peptides, glycoproteins, glycopeptides, glycolipids, polysaccharides, oligosaccharides, nucleic acids, bioorganic molecules, peptidomimetics, small molecules, pharmacological agents and their metabolites, transcriptional and translation control sequences, and the like. In one embodiment of the invention, the TAM antagonist is an antibody, especially an anti-TAM cell surface marker antibody which binds human TAM.

The term “ATM antagonist” when used herein refers to a molecule which binds to ATM and inhibits or substantially reduces a biological activity of ATM. Non-limiting examples of ATM antagonists include antibodies, proteins, peptides, glycoproteins, glycopeptides, glycolipids, polysaccharides, oligosaccharides, nucleic acids, bioorganic molecules, peptidomimetics, small molecules, pharmacological agents and their metabolites, transcriptional and translation control sequences, and the like. In one embodiment of the invention, the TAM antagonist is an antibody, especially an anti-ATM cell surface marker antibody which binds human ATM.

The term “F4/80 antagonist” when used herein refers to a molecule which binds to F4/80 and inhibits or substantially reduces a biological activity of F4/80. Non-limiting examples of F4/80 antagonists include antibodies, proteins, peptides, glycoproteins, glycopeptides, glycolipids, polysaccharides, oligosaccharides, nucleic acids, bioorganic molecules, peptidomimetics, small molecules, pharmacological agents and their metabolites, transcriptional and translation control sequences, and the like. In one embodiment of the invention, the F4/80 antagonist is an antibody, especially an anti-F4/80 antibody which binds human F4/80.

The term “CD11c antagonist” when used herein refers to a molecule which binds to CD11c and inhibits or substantially reduces a biological activity of CD11c. Non-limiting examples of CD11c antagonists include antibodies, proteins, peptides, glycoproteins, glycopeptides, glycolipids, polysaccharides, oligosaccharides, nucleic acids, bioorganic molecules, peptidomimetics, small molecules, pharmacological agents and their metabolites, transcriptional and translation control sequences, and the like. In one embodiment of the invention, the CD11c antagonist is an antibody, especially an anti-CD11c antibody which binds human CD11c.

The term “langerin antagonist” when used herein refers to a molecule which binds to langerin (preferably human langerin) and inhibits or substantially reduces a biological activity of langerin. Non-limiting examples of langerin antagonists include antibodies, proteins, peptides, glycoproteins, glycopeptides, glycolipids, polysaccharides, oligosaccharides, nucleic acids, bioorganic molecules, peptidomimetics, small molecules, pharmacological agents and their metabolites, transcriptional and translation control sequences, and the like. In one embodiment of the invention, the langerin antagonist is an antibody, especially an anti-langerin antibody which binds human langerin intracellularly. In another embodiment of the invention, the langerin antagonist is a small molecule that binds human langerin.

The term “agonist” refers to a molecule capable of stimulating, activating, or otherwise enhancing the activities of a protein of the invention including its binding to one or more receptors in the case of a ligand or binding to one or more ligands in case of a receptor. Agonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Agonists also include small molecule activators of a protein of the invention, and fusion proteins, receptor molecules and derivatives which bind specifically to a protein and in so doing enhance the protein's activity to, e.g., bind to its target, agonist variants of the protein, antisense molecules directed to an inhibitor of the protein of the invention, RNA aptamers specific for an inhibitor of the protein of the invention, and ribozymes against an inhibitor of a protein of the invention. The term “IRTM agonist” refers to a molecule capable of stimulating, activating, or otherwise enhancing the activities of IRTM, e.g., by binding to one or more IRTM receptors and stimulating IRTM activity, or by binding to one or more IRTM inhibitors and preventing interaction of the inhibitor with IRTM. Agonists include, but are not limited to, antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, small molecules, fusion proteins, receptor molecules and derivatives, as well as antisense molecules, RNA aptamers, and ribozymes directed to an IRTM inhibitor.

The term “TAM agonist” refers to a molecule capable of stimulating, activating, or otherwise enhancing the activities of TAM, e.g., by binding to one or more TAM receptors and stimulating TAM activity, or by binding to one or more TAM inhibitors and preventing interaction of the inhibitor with TAM. Agonists include, but are not limited to, antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, small molecules, fusion proteins, receptor molecules and derivatives, as well as antisense molecules, RNA aptamers, and ribozymes directed to a TAM inhibitor.

The term “ATM agonist” refers to a molecule capable of stimulating, activating, or otherwise enhancing the activities of ATM, e.g., by binding to one or more ATM receptors and stimulating ATM activity, or by binding to one or more ATM inhibitors and preventing interaction of the inhibitor with ATM. Agonists include, but are not limited to, antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, small molecules, fusion proteins, receptor molecules and derivatives, as well as antisense molecules, RNA aptamers, and ribozymes directed to a ATM inhibitor.

A “native sequence” polypeptide comprises a polypeptide having the same amino acid sequence as a polypeptide derived from nature. Thus, a native sequence polypeptide can have the amino acid sequence of naturally occurring polypeptide from any mammal. Such native sequence polypeptide can be isolated from nature or can be produced by recombinant or synthetic means. The term “native sequence” polypeptide specifically encompasses naturally occurring truncated or secreted forms of the polypeptide (e.g., an extracellular domain sequence), naturally occurring variant forms (e.g., alternatively spliced forms) and naturally occurring allelic variants of the polypeptide.

A “polypeptide chain” is a polypeptide wherein each of the domains thereof is joined to other domain(s) by peptide bond(s), as opposed to non-covalent interactions or disulfide bonds.

A polypeptide “variant” means a biologically active polypeptide having at least about 80% amino acid sequence identity with the corresponding native sequence polypeptide. Such variants include, for instance, polypeptides wherein one or more amino acid (naturally occurring amino acid and/or a non-naturally occurring amino acid) residues are added, or deleted, at the N- and/or C-terminus of the polypeptide. Ordinarily, a variant will have at least about 80% amino acid sequence identity, or at least about 90% amino acid sequence identity, or at least about 95% or more amino acid sequence identity with the native sequence polypeptide. Variants also include polypeptide fragments (e.g., subsequences, truncations, etc.), typically biologically active, of the native sequence.

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2. The ALIGN-2 sequence comparison computer program was authored by Genentech, Inc. has been filed with user documentation in the U.S. Copyright Office, Washington D.C., 20559, where it is registered under U.S. Copyright Registration No. TXU510087, and is publicly available through Genentech, Inc., South San Francisco, Calif. The ALIGN-2 program should be compiled for use on a UNIX operating system, e.g., digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

The term “protein variant” as used herein refers to a variant as described above and/or a protein which includes one or more amino acid mutations in the native protein sequence. Optionally, the one or more amino acid mutations include amino acid substitution(s). Protein and variants thereof for use in the invention can be prepared by a variety of methods well known in the art. Amino acid sequence variants of a protein can be prepared by mutations in the protein DNA. Such variants include, for example, deletions from, insertions into or substitutions of residues within the amino acid sequence of protein. Any combination of deletion, insertion, and substitution may be made to arrive at the final construct having the desired activity. The mutations that will be made in the DNA encoding the variant must not place the sequence out of reading frame and preferably will not create complementary regions that could produce secondary mRNA structure.

The protein variants optionally are prepared by site-directed mutagenesis of nucleotides in the DNA encoding the native protein or phage display techniques, thereby producing DNA encoding the variant, and thereafter expressing the DNA in recombinant cell culture.

While the site for introducing an amino acid sequence variation is predetermined, the mutation per se need not be predetermined. For example, to optimize the performance of a mutation at a given site, random mutagenesis may be conducted at the target codon or region and the expressed protein variants screened for the optimal combination of desired activity. Techniques for making substitution mutations at predetermined sites in DNA having a known sequence are well-known, such as, for example, site-specific mutagenesis. Preparation of the protein variants described herein can be achieved by phage display techniques, such as those described in the PCT publication WO 00/63380.

After such a clone is selected, the mutated protein region may be removed and placed in an appropriate vector for protein production, generally an expression vector of the type that may be employed for transformation of an appropriate host.

Amino acid sequence deletions generally range from about 1 to 30 residues, optionally 1 to 10 residues, optionally 1 to 5 residues or less, and typically are contiguous. Amino acid sequence insertions include amino- and/or carboxyl-terminal fusions of from one residue to polypeptides of essentially unrestricted length as well as intrasequence insertions of single or multiple amino acid residues. Intrasequence insertions (i.e., insertions within the native protein sequence) may range generally from about 1 to 10 residues, optionally 1 to 5, or optionally 1 to 3. An example of a terminal insertion includes a fusion of a signal sequence, whether heterologous or homologous to the host cell, to the N-terminus to facilitate the secretion from recombinant hosts.

Additional protein variants are those in which at least one amino acid residue in the native protein has been removed and a different residue inserted in its place. Such substitutions may be made in accordance with those shown in Table 1. Protein variants can also include unnatural amino acids as described herein.

Amino acids may be grouped according to similarities in the properties of their side chains (in A. L. Lehninger, in Biochemistry, second ed., pp. 73-75, Worth Publishers, New York (1975)):

non-polar: Ala (A), Val (V), Leu (L), Ile (I), Pro (P), Phe (F), Trp (W), Met (M)

uncharged polar: Gly (G), Ser (S), Thr (T), Cys (C), Tyr (Y), Asn (N), Gln (Q)

acidic: Asp (D), Glu (E)

basic: Lys (K), Arg (R), His (H)

Alternatively, naturally occurring residues may be divided into groups based on common side-chain properties:

hydrophobic: Norleucine, Met, Ala, Val, Leu, Ile;

neutral hydrophilic: Cys, Ser, Thr, Asn, Gln;

acidic: Asp, Glu;

basic: His, Lys, Arg;

residues that influence chain orientation: Gly, Pro;

aromatic: Trp, Tyr, Phe.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; | Tyr |
| | Ile; Ala; Tyr | |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

“Naturally occurring amino acid residues” (i.e. amino acid residues encoded by the genetic code) may be selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include, e.g., norleucine, ornithine, norvaline, homoserine and other amino acid residue analogues such as those described in Ellman et al. Meth. Enzym. 202:301-336 (1991) & US Patent application publications 20030108885 and 20030082575. Briefly, these procedures involve activating a suppressor tRNA with a non-naturally occurring amino acid residue followed by in vitro or in vivo transcription and translation of the RNA. See, e.g., US Patent application publications 20030108885 and 20030082575; Noren et al. Science 244:182 (1989); and, Ellman et al., supra.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In certain embodiments, the polypeptide will be purified (1) to greater than 95% by weight of polypeptide as determined by the Lowry method, or more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or silver stain. Isolated polypeptide includes the polypeptide in situ within recombinant cells since at least one component of the polypeptide's natural environment will not be present. Ordinarily, however, isolated polypeptide will be prepared by at least one purification step.

The term “antibody” is used in the broadest sense and specifically covers monoclonal antibodies (including full length or intact monoclonal antibodies), polyclonal antibodies, multivalent antibodies, multispecific antibodies (e.g., bispecific antibodies) formed from at least two intact antibodies, and antibody fragments (see below) so long as they exhibit the desired biological activity.

Unless indicated otherwise, the expression “multivalent antibody” is used throughout this specification to denote an antibody comprising three or more antigen binding sites. The multivalent antibody is typically engineered to have the three or more antigen binding sites and is generally not a native sequence IgM or IgA antibody.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057 1062 (1995); and U.S. Pat. No. 5,641,870).

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. Monoclonal antibodies are highly specific, being directed against a single antigen. In certain embodiments, a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations that typically include different antibodies directed against different determinants (epitopes), each monoclonal antibody is directed against a single determinant on the antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1991); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1: 105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409. See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. PNAS (USA) 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cellular toxicity.

The term “hypervariable region,” “HVR,” or “HV,” when used herein refers to the amino acid residues of an antibody which are responsible for antigen-binding. For example, the term hypervariable region refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact | |
|---|---|---|---|---|---|
| L1 | L24-L34 | L24-L34 | L26-L32 | L30-L36 | |
| L2 | L50-L56 | L50-L56 | L50-L52 | L46-L55 | |
| L3 | L89-L97 | L89-L97 | L91-L96 | L89-L96 | |
| H1 | H31-H35B | H26-H35B | H26-H32 | H30-H35B | (Kabat Numbering) |
| H1 | H31-H35 | H26-H35 | H26-H32 | H30-H35 | (Chothia Numbering) |
| H2 | H50-H65 | H50-H58 | H53-H55 | H47-H58 | |
| H3 | H95-H102 | H95-H102 | H96-H101 | H93-H101 | |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework Region” or “FR” residues are those variable domain residues other than the hypervariable region residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

Throughout the present specification and claims, the Kabat numbering system is generally used when referring to a residue in the variable domain (approximately, residues 1-107 of the light chain and residues 1-113 of the heavy chain) (e.g, Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, Md. (1991) expressly incorporated herein by reference). Unless stated otherwise herein, references to residues numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG, (including non-A and A allotypes), IgG₂, IgG₃, IgG₄, IgA₁, and IgA₂. The heavy chain constant domains that correspond to the different classes of immunoglobulins are called α, δ, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The “light chains” of antibodies (immunoglobulins) from any vertebrate species can be assigned to one of two clearly distinct types, called kappa (K) and lambda (λ), based on the amino acid sequences of their constant domains.

The term “Fc region” is used to define the C-terminal region of an immunoglobulin heavy chain which may be generated by papain digestion of an intact antibody. The Fc region may be a native sequence Fc region or a variant Fc region. Although the boundaries of the Fc region of an immunoglobulin heavy chain might vary, the human IgG heavy chain Fc region is usually defined to stretch from an amino acid residue at about position Cys226, or from about position Pro230, to the carboxyl-terminus of the Fc region. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. The Fc region of an immunoglobulin generally comprises two constant domains, a CH2 domain and a CH3 domain, and optionally comprises a CH4 domain. Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

By “Fc region chain” herein is meant one of the two polypeptide chains of an Fc region.

The “CH2 domain” of a human IgG Fc region (also referred to as “Cg2” domain) usually extends from an amino acid residue at about position 231 to an amino acid residue at about position 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22: 161-206 (1985). The CH2 domain herein may be a native sequence CH2 domain or variant CH2 domain.

The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protruberance” in one chain thereof and a corresponding introduced “cavity” in the other chain thereof, see U.S. Pat. No. 5,821,333, expressly incorporated herein by reference). Such variant CH3 domains may be used to make multispecific (e.g. bispecific) antibodies as herein described.

“Hinge region” is generally defined as stretching from about Glu216, or about Cys226, to about Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions. The hinge region herein may be a native sequence hinge region or a variant hinge region. The two polypeptide chains of a variant hinge region generally retain at least one cysteine residue per polypeptide chain, so that the two polypeptide chains of the variant hinge region can form a disulfide bond between the two chains. The preferred hinge region herein is a native sequence human hinge region, e.g. a native sequence human IgG1 hinge region.

A “functional Fc region” possesses at least one “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In certain embodiments, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide, e.g. from about one to about ten amino acid substitutions, and preferably from about one to about five amino acid substitutions in a native sequence Fc region or in the Fc region of the parent polypeptide. The variant Fc region herein will typically possess, e.g., at least about 80% sequence identity with a native sequence Fc region and/or with an Fc region of a parent polypeptide, or at least about 90% sequence identity therewith, or at least about 95% sequence or more identity therewith.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. In certain embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells and neutrophils; with PBMCs and NK cells being generally preferred. The effector cells may be isolated from a native source thereof, e.g. from blood or PBMCs as described herein.

“Fc receptor” or “FcR” describes a receptor that binds to the Fc region of an antibody. In some embodiments, an FcR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see, e.g., Daëron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and serum half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See also, e.g., Shields et al. J. Biol. Chem. 9(2):6591-6604 (2001).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1 and WO 1999/51642. See also, e.g., Idusogie et al. J. Immunol. 164: 4178-4184 (2000).

An “affinity matured” antibody is one with one or more alterations in one or more CDRs thereof which result an improvement in the affinity of the antibody for antigen, compared to a parent antibody which does not possess those alteration(s). In one embodiment, an affinity matured antibody has nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “flexible linker” herein refers to a peptide comprising two or more amino acid residues joined by peptide bond(s), and provides more rotational freedom for two polypeptides (such as two Fd regions) linked thereby. Such rotational freedom allows two or more antigen binding sites joined by the flexible linker to each access target antigen(s) more efficiently. Examples of suitable flexible linker peptide sequences include gly-ser, gly-ser-gly-ser, ala-ser, and gly-gly-gly-ser.

A “dimerization domain” is formed by the association of at least two amino acid residues (generally cysteine residues) or of at least two peptides or polypeptides (which may have the same, or different, amino acid sequences). The peptides or polypeptides may interact with each other through covalent and/or non-covalent association(s). Examples of dimerization domains herein include an Fc region; a hinge region; a CH3 domain; a CH4 domain; a CH1-CL pair; an “interface” with an engineered “knob” and/or “protruberance” as described in U.S. Pat. No. 5,821,333, expressly incorporated herein by reference; a leucine zipper (e.g. ajun/fos leucine zipper, see Kostelney et al., J. Immunol., 148: 1547-1553 (1992); or a yeast GCN4 leucine zipper); an isoleucine zipper; a receptor dimer pair (e.g., interleukin-8 receptor (IL-8R); and integrin heterodimers such as LFA-1 and GPIIIb/IIIa), or the dimerization region(s) thereof; dimeric ligand polypeptides (e.g. nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF); see Arakawa et al. J. Biol. Chem. 269(45): 27833-27839 (1994) and Radziejewski et al. Biochem. 32(48): 1350 (1993)), or the dimerization region(s) thereof; a pair of cysteine residues able to form a disulfide bond; a pair of peptides or polypeptides, each comprising at least one cysteine residue (e.g. from about one, two or three to about ten cysteine residues) such that disulfide bond(s) can form between the peptides or polypeptides (hereinafter “a synthetic hinge”); and antibody variable domains. In one embodiment, a dimerization domain herein is an Fc region or a hinge region.

A “functional antigen binding site” of an antibody is one which is capable of binding a target antigen. The antigen binding affinity of the antigen binding site is not necessarily as strong as the parent antibody from which the antigen binding site is derived, but the ability to bind antigen must be measurable using any one of a variety of methods known for evaluating antibody binding to an antigen. Moreover, the antigen binding affinity of each of the antigen binding sites of a multivalent antibody herein need not be quantitatively the same. For the multimeric antibodies herein, the number of functional antigen binding sites can be evaluated using ultracentrifugation analysis. According to this method of analysis, different ratios of target antigen to multimeric antibody are combined and the average molecular weight of the complexes is calculated assuming differing numbers of functional binding sites. These theoretical values are compared to the actual experimental values obtained in order to evaluate the number of functional binding sites.

An antibody having a “biological characteristic” of a designated antibody is one which possesses one or more of the biological characteristics of that antibody which distinguish it from other antibodies that bind to the same antigen.

In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

Administration “in combination with” one or more further therapeutic agents includes simultaneous (concurrent) and/or consecutive administration in any order.

“Mammal” for purposes of treatment refers to any animal classified as a mammal, including humans, domestic and farm animals, and zoo, sports, or pet animals, such as dogs, horses, cats, cows, sheep, pigs, etc. Typically, the mammal is a human.

A “disorder” is any condition that would benefit from treatment with the molecules of the invention. This includes chronic and acute disorders or diseases including those pathological conditions which predispose the mammal to the disorder in question. Disorders include cell proliferative disorders, angiogenic disorders, and inflammatory, angiogenic and immunologic disorders (including, but not limited to, autoimmune disorders).

The terms “cell proliferative disorder” and “proliferative disorder” refer to disorders that are associated with some degree of abnormal cell proliferation and/or hypertrophy. In one embodiment, the cell proliferative disorder is cancer.

The terms “inflammatory disorder” and “immune disorder” refer to or describe disorders caused by aberrant immunologic mechanisms and/or aberrant cytokine signaling (e.g., aberrant interferon signaling). Examples of inflammatory and immune disorders include, but are not limited to, autoimmune diseases, immunologic deficiency syndromes, and hypersensitivity.

The term “inflammatory disorder” refers to a disease or disorder based on or related to an inflammatory condition. Inflammatory disorders include, but are not limited to, autoimmune disorders, hyperglycemic disorders, and disorders associated with insulin resistance.

The term “autoimmune disorder” refers to a non-malignant disease or disorder arising from and directed against an individual's own tissues. Autoimmune disorders are typically characterized by the failure of autoreactive immune cells to be destroyed by the immune system; autoreactive lymphocytes have been identified that overexpress or otherwise have increased activity of pro-survival apoptotic factors or have reduced expression or activity of pro-apoptotic factors. The autoimmune disorders herein specifically exclude malignant or cancerous diseases or conditions, especially excluding B cell lymphoma, acute lymphoblastic leukemia (ALL), chronic lymphocytic leukemia (CLL), Hairy cell leukemia and chronic myeloblastic leukemia. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); diabetes mellitus (e.g. Type I diabetes mellitus or insulin dependent diabetes mellitus); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia, etc.

The term “hyperglycemic disorder” includes, but is not limited to, diabetes and related diseases/disorders, including, but not limited to, hyperlipidemia and obesity caused by a hyperglycemic disorder.

The term “disorder associated with insulin resistance” includes, but is not limited to, insulin resistance, polycystic ovary syndrome, coronary artery disease and peripheral vascular disease.

The term “effective amount” or “therapeutically effective amount” refers to an amount of a drug effective to treat a disease or disorder in a mammal. In the case of cancer, the effective amount of the drug may reduce the number of cancer cells; reduce the tumor size; inhibit (i.e., slow to some extent and typically stop) cancer cell infiltration into peripheral organs; inhibit (i.e., slow to some extent and typically stop) tumor metastasis; inhibit, to some extent, tumor growth; allow for treatment of the tumor, and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

“Treatment” refers to both therapeutic treatment and prophylactic or preventative measures. Those in need of treatment include those already with the disorder as well as those in which the disorder is to be prevented. In certain embodiments of the invention, treatment can refer to a suppression of tumor growth or to a suppression of an autoimmune disorder.

The term “biological activity” and “biologically active” with regard to a polypeptide of the invention refer to the ability of a molecule to specifically bind to a target and regulate cellular responses, e.g., proliferation, migration, etc. Cellular responses also include those mediated through a receptor, including, but not limited to, migration and/or proliferation. In this context, the term “modulate” includes both promotion and inhibition.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include kidney or renal cancer, breast cancer, colon cancer, rectal cancer, colorectal cancer, lung cancer including small-cell lung cancer, non-small cell lung cancer, adenocarcinoma of the lung and squamous carcinoma of the lung, squamous cell cancer (e.g. epithelial squamous cell cancer), cervical cancer, ovarian cancer, prostate cancer, liver cancer, bladder cancer, cancer of the peritoneum, hepatocellular cancer, gastric or stomach cancer including gastrointestinal cancer, gastrointestinal stromal tumors (GIST), pancreatic cancer, head and neck cancer, glioblastoma, retinoblastoma, astrocytoma, thecomas, arrhenoblastomas, hepatoma, hematologic malignancies including non-Hodgkins lymphoma (NHL), multiple myeloma and acute hematologic malignancies, endometrial or uterine carcinoma, endometriosis, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, melanoma, skin carcinomas, Schwannoma, oligodendroglioma, neuroblastomas, rhabdomyosarcoma, osteogenic sarcoma, leiomyosarcomas, urinary tract carcinomas, thyroid carcinomas, Wilm's tumor, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. “Tumor”, as used herein, refers to all neoplastic cell growth and proliferation, whether malignant or benign, and all pre-cancerous and cancerous cells and tissues.

The term “cytotoxic agent” as used herein refers to a substance that inhibits or prevents the function of cells and/or causes destruction of cells. The term is intended to include radioactive isotopes (e.g., ²¹¹At, ¹³¹I, ¹²⁵I, ⁹⁰Y, ¹⁸⁶Re, ¹⁸⁸Re, ¹⁵³m, ²¹²Bi, ³²P and radioactive isotopes of Lu), chemotherapeutic agents, and toxins such as small molecule toxins or enzymatically active toxins of bacterial, fungal, plant or animal origin, including fragments and/or variants thereof.

A “growth inhibitory agent” when used herein refers to a compound or composition which inhibits growth of a cell in vitro and/or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), TAXOL®, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINEL®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chlorambucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovorin; vinorelbine (NAVELBINE®); novantrone; edatrexate; daunomycin; aminopterin; ibandronate; topoisomerase inhibitor RFS 2000; difluoromethylomithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl)benzenesulfonamide; and pharmaceutically acceptable salts, acids or derivatives of any of the above.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factors (e.g., VEGF, VEGF-B, VEGF-C, VEGF-D, VEGF-E); placental derived growth factor (PlGF); platelet derived growth factors (PDGF, e.g., PDGFA, PDGFB, PDGFC, PDGFD); integrin; thrombopoietin (TPO); nerve growth factors such as NGF-alpha; platelet-growth factor; transforming growth factors (TGFs) such as TGF-alpha and TGF-beta; insulin-like growth factor-I and -II; erythropoietin (EPO); osteoinductive factors; interferons such as interferon-alpha, -beta and -gamma, colony stimulating factors (CSFS) such as macrophage-CSF (M-CSF); granulocyte-macrophage-CSF (GM-CSF); and granulocyte-CSF (G-CSF); interleukins (ILs) such as IL-1, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20-IL-30; secretoglobin/uteroglobin; oncostatin M (OSM); a tumor necrosis factor such as TNF-alpha or TNF-beta; and other polypeptide factors including LIF and kit ligand (KL). As used herein, the term cytokine includes proteins from natural sources or from recombinant cell culture and biologically active equivalents of the native sequence cytokines.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

An “angiogenic factor or agent” is a growth factor which stimulates the development of blood vessels, e.g., promotes angiogenesis, endothelial cell growth, stability of blood vessels, and/or vasculogenesis, etc. For example, angiogenic factors, include, but are not limited to, e.g., VEGF and members of the VEGF family, PlGF, PDGF family, fibroblast growth factor family (FGFs), TIE ligands (Angiopoietins), ephrins, ANGPTL3, ANGPTL4, etc. It would also include factors that accelerate wound healing, such as growth hormone, insulin-like growth factor-I (IGF-I), VIGF, epidermal growth factor (EGF), CTGF and members of its family, and TGF-α and TGF-β. See, e.g., Klagsbrun and D'Amore, Annu. Rev. Physiol., 53:217-39 (1991); Streit and Detmar, Oncogene, 22:3172-3179 (2003); Ferrara & Alitalo, Nature Medicine 5(12): 1359-1364 (1999); Tonini et al., Oncogene, 22:6549-6556 (2003) (e.g., Table 1 listing angiogenic factors); and, Sato Int. J. Clin. Oncol., 8:200-206 (2003).

An “anti-angiogenesis agent” or “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. For example, an anti-angiogenesis agent is an antibody or other antagonist to an angiogenic agent as defined above, e.g., antibodies to VEGF, antibodies to VEGF receptors, small molecules that block VEGF receptor signaling (e.g., PTK787/ZK2284, SU6668, SUTENT/SU11248 (sunitinib malate), AMG706). Anti-angiogenesis agents also include native angiogenesis inhibitors, e.g., angiostatin, endostatin, etc. See, e.g., Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12):1359-1364 (1999); Tonini et al., Oncogene, 22:6549-6556 (2003) (e.g., Table 2 listing antiangiogenic factors); and, Sato Int. J. Clin. Oncol., 8:200-206 (2003) (e.g., Table 1 lists anti-angiogenic agents used in clinical trials).

The term “immunosuppressive agent” as used herein refers to substances that act to suppress or mask the immune system of the mammal being treated herein, including to modulate inflammation. This includes, but is not limited to, substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); nonsteroidal antiinflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, and dexamethasone; dihydrofolate reductase inhibitors such as methotrexate (oral or subcutaneous); hydroxycloroquine; sulfasalazine; leflunomide; cytokine or cytokine receptor antibodies including anti-interferon-alpha, -beta, or -gamma antibodies, anti-tumor necrosis factor-alpha antibodies (infliximab or adalimumab), anti-TNF-alpha immunoadhesin (etanercept), anti-tumor necrosis factor-beta antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 1990/08187 published Jul. 26, 1990); streptokinase; TGF-beta; streptodornase; RNA or DNA from the host; FK506; RS-61443; deoxyspergualin; rapamycin; T-cell receptor (Cohen et al., U.S. Pat. No. 5,114,721); T-cell-receptor fragments (Offner et al., Science, 251: 430-432 (1991); WO 1990/11294; Taneway, Nature, 341: 482 (1989); and WO 1991/01133); and T-cell-receptor antibodies (EP 340,109) such as T10B9.

Examples of “nonsteroidal anti-inflammatory drugs” or “NSAIDs” are acetylsalicylic acid, ibuprofen, naproxen, indomethacin, sulindac, tolmetin, including salts and derivatives thereof, etc.

The word “label” when used herein refers to a detectable compound or composition which is conjugated directly or indirectly to the polypeptide. The label may be itself be detectable (e.g., radioisotope labels or fluorescent labels) or, in the case of an enzymatic label, may catalyze chemical alteration of a substrate compound or composition which is detectable. An “isolated” nucleic acid molecule is a nucleic acid molecule that is identified and separated from at least one contaminant nucleic acid molecule with which it is ordinarily associated in the natural source of the polypeptide nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the polypeptide where, for example, the nucleic acid molecule is in a chromosomal location different from that of natural cells.

### Methods of the Invention

The invention identifies certain novel properties and activities of IRTM, particularly TAM and ATM, that may be exploited using the methods of the invention for therapeutic purposes. Chronic inflammation is a common feature of many diseases with distinct etiopathogenic origins, such as cancer, type II diabetes and atherosclerosis. Recently, macrophages have been directly implicated in the pathogenesis of these disorders (Mantovani et al., Immunol. Today 13:265-70, 1992; Pollard, Nat. Rev. Cancer 4: 71-8, 2004; Arkan et al., Nat. Med. 11: 191-8, 2005; Lumeng et al., J. Clin Invest. 117: 175-84, 2007; Liang et al., Circ. Res. 100: 1546-55, 2007; Choudhury, Nat Clin Pract Cardiovasc Med 2 (6): 309-15, 2005). In order to understand their function in tumors, experiments were performed to characterize TAM in the PyMT^{tg} model, which recapitulates many aspects of human infiltrating ductal carcinoma of the breast (Lin et al., Am J. Pathol 163: 2113-26, 2003). In order to understand their function in other inflammatory disorders, including but not limited to type II diabetes and insulin resistance, experiments were performed to characterize ATM from mice fed a chronic high fat diet.

As shown herein and as known in the art, TAM are commonly found in tumor immune cell infiltrates. High levels of TAM have been correlated with poor prognosis in human tumors, and inhibition of TAM differentiation in a genetic model of mammary cancer was shown to reduce the rate of tumor progression and metastasis (Lin et al., 2001). It has been proposed that TAM contribute to tumor growth by producing angiogenic factors such as VEGF, thus increasing vascularization of tumors (Leek et al. 1996; Lin et al. 2006). Others have suggested that TAM may have an indirect role in inducing tolerance by secreting certain cytokines that inhibit the maturation of professional antigen presenting cells (e.g., dendritic cells) in tumors, thereby impairing the ability of such cells to present aberrant tumor cells to the immune system so that an effective immune response against the tumor is not raised (Mantovanti et al. 2002; Pollard et al., 2003).

Herein, it is shown that in addition to the above activities, TAM also induce two specific CD4⁺ T regulatory cell subsets: FoxP3⁺ T regulatory cells, and IL-10⁺ Trl cells. Incubation of TAM with naïve T cells induced proliferation of both IL-10⁺ Trl and FoxP3⁺ T regulatory cells and production of the cytokines expected to be produced from those cell types (IL-10 and IL-17, and little to no IL-2 or IL-4), whereas incubation of bmDC with naïve T cells did not have the same effect. This induction by TAM was inhibited by the inclusion of TGFβRII in the culture, suggesting that TGFβ is important for TAM-induced induction of those cell types. Elevated levels of these regulatory T cell subsets have previously been correlated with solid tumors and reduced overall breast cancer survival rates (Leong et al., 2006; Liyanage et al., 2002; Marshall et al., 2004; Seo et al., 2001; Curiel et al., 2004; and Bates et al., 2006). TAM were also shown to induce inflammatory TH₁₇ cells in vitro, correlating with the increased numbers of TH₁₇ cells observed in the draining lymph nodes of PyMT^{tg} tumor-bearing mice. While TAM were similar to bmDC or peritoneal macrophages in their ability to induce IL-17⁺ T cells, neither of those other types of macrophages were able to induce both regulatory and pro-inflammatory T cell subsets. The profile of T cells induced in vitro by TAM was identical to the types of T cells increased in mammary tumor-bearing animals. The invention provides methods of modulating TAM-mediated induction of IL-10⁺ Trl and FoxP3⁺ T regulatory cells and inflammatory TH₁₇ cells in vitro and in vivo to modulate the initiation, progression, or severity of tumor growth and activity. The invention also provides methods of detecting tumor formation, progression, and/or staging a tumor by detecting the presence, amount, and/or activity of TAM.

In humans as well as rodents, obesity is associated with an increased infiltration of adipose tissue macrophages (ATM). Obesity has been correlated with cardiovascular disease, diabetes, kidney disease and some types of cancers (Flegal et al., JAMA 298(17): 2028-37, 2007). Obesity is also associated with chronic inflammation that predisposes the subject to insulin resistance and the development of type II diabetes. Several recent studies have demonstrated that ATM produce inflammatory cytokines, which can block insulin action in adipocytes and have been proposed as contributors to systemic insulin resistance (Weisberg et al., J. Clin. Invest. 112: 1796-808, 2003; Arkan et al., Nat. Med. 11: 191-8, 2005; Neels and Olefsky, J. Clin. Invest. 116: 33-5, 2006; Lumeng et al., J. Clin. Invest. 117: 175-84, 2007). Herein, it is shown that, like TAM, ATM induce two specific CD4⁺ T regulatory cell subsets: FoxP3⁺ T regulatory cells, and IL-10⁺ Trl cells, as well as inducing inflammatory TH₁₇ cells.

In normal systems, T regulatory cells play a key role in inducing tolerogenesis by suppressing conventional T cells and downregulating their activity. T regulatory cells have been shown to be therapeutic in a variety of experimental autoimmune disorder settings (see Suri-Payer and Fritzsching, Springer Semin. Immun. (2006) 28:3-16). The ability to selectively induce T regulatory cells in certain disease states, such as immune disorders, particularly inflammatory and autoimmune disorders, where such cells are of therapeutic value, is of clear therapeutic value. The invention also provides methods of initiating and/or stimulating IL-10⁺ Trl and FoxP3⁺ T regulatory cell induction by modulating TAM or ATM presence or activity, which methods may be used to modulate the initiation, progression, or severity of inflammatory and autoimmune disorders.

TH₁₇ cells are also present at elevated levels in draining lymph nodes from tumors and adipose tissue. The role of TH₁₇ cells in the pathology of cancer or type II diabetes is not yet clear. IL-17 may promote tumor growth indirectly, by inducing expression of other proinflammatory mediates such as TNFα, IL-1β and IL-6. IL-17, which like TNFα activates NF-κB, may also act directly as a pro-survival and angiogenic factor for tumors (Lin, and Karin, J Clin Invest 117 (5): 1175-83, 2007; Takahashi, et al., Immunol Lett 98 (2): 189-93, 2005; Numasaki et al., J Immunol 175 (9): 6177-89, 2005). s Interestingly ATM and TAM, but not control macrophages, induced both T regulatory and TH₁₇ cells in vitro and both of these populations are increased in tumor-bearing and obese mice. These data again emphasize that pro- and anti-inflammatory mechanisms co-exist thus leading to a state of chronic inflammation. Such chronic inflammation may be modulated by modulating TAM or ATM presence or activity.

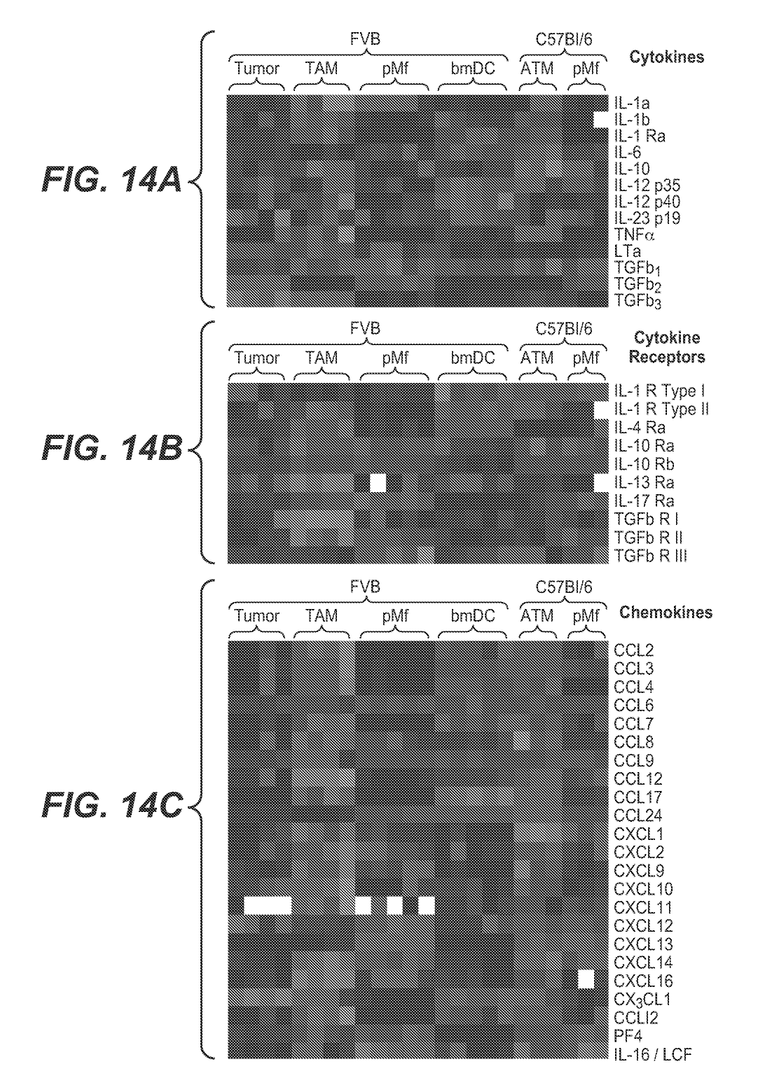

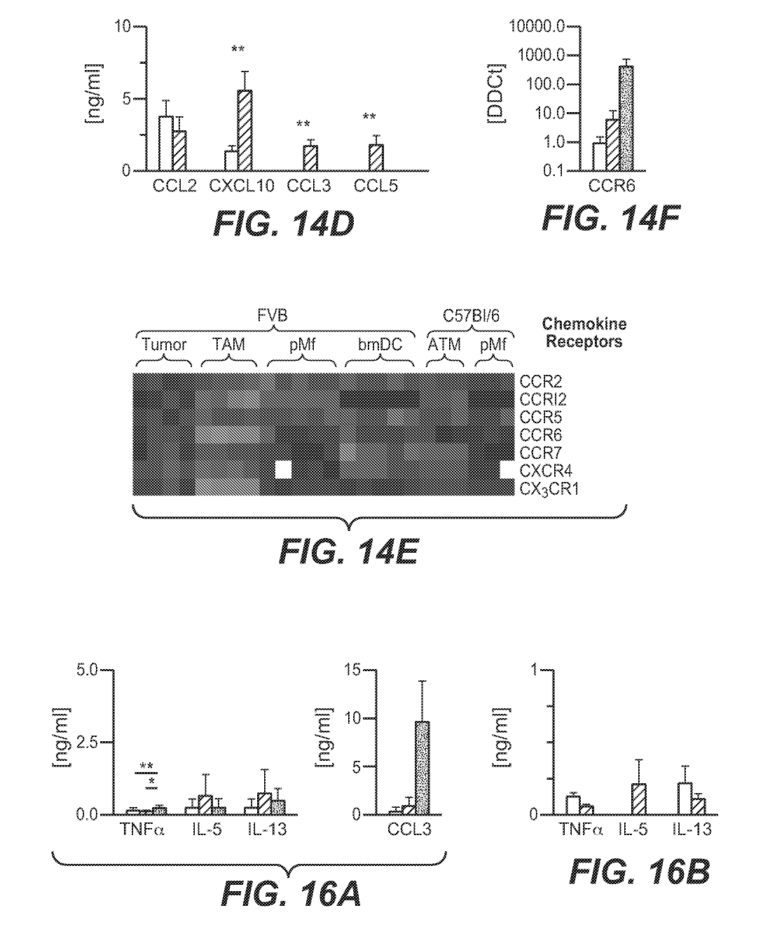

The work described herein provides further characterization of TAM and ATM. For example, TAM are shown to have certain properties of peritoneal macrophages and bmDC in terms of cytokine/chemokine production and cell surface markers. As shown herein, TAM express both the macrophage marker F4/80 and the dendritic cell markers langerin and CD11c. ATM express both the macrophage marker F4/80 and the dendritic cell marker CD11c. Furthermore, each of TAM and ATM express different subsets of chemokines, cytokines, chemokine receptors, and cytokine receptors (see FIGS. 14A-C and E), which can individually or collectively be used as markers for the presence and/or activity of TAM or ATM. The invention provides methods of identifying/detecting and isolating TAM and/or ATM from a population of cells or a sample containing cells by contacting the population or sample with one or more reagents to detect TAM and/or ATM markers and optionally separating the TAM and/or ATM from the rest of the population or sample.

The invention also provides methods of modulating TAM and/or ATM. For example, TAM and/or ATM activity or function may be blocked by selectively removing or killing TAM and/or ATM. One method to accomplish this is to specifically target TAM and/or ATM (i.e., by targeting only cells simultaneously bearing both macrophage-specific and DC-specific cell surface markers) with a TAM and/or ATM-binding agent and selectively removing the specifically targeted cells from the population. For example, a bispecific antibody or fragment thereof that specifically recognizes both F4/80 and CD11c may be used to specifically bind TAM and/or ATM and then separate TAM and/or ATM from the remaining cell population/sample by, e.g., protein A chromatography or any other method of antibody capture and separation well known in the art including, but not limited to, FACS, affinity chromatography, and magnetic cell sorting. In another method, one can specifically target TAM and/or ATM (i.e., by targeting only cells simultaneously bearing both macrophage-specific and DC-specific cell surface markers) and selectively kill the specifically targeted cells from the population. For example, the same bispecific antibody (or fragment thereof) scenario as described above may be employed, but the antibody may be additionally conjugated with a cytotoxic molecule, or effector function of the antibody itself may be sufficient to trigger clearance and destruction of the TAM and/or ATM bound to the antibody. It is not necessary to use bispecific or other multispecific antibodies; one of ordinary skill in the art will recognize that the same goal may be accomplished with two or more separate antibodies or fragments thereof or other binding molecules that provide some means to be selectively pulled from a mixture of cells while still remaining associated with TAM and/or ATM. Appropriate TAM and/or ATM cell surface markers for such selection may be found, e.g., in FIGS. 14B and 14E and include, but are not limited to, IL-1R type I, IL4Rα, IL-13Rα; IL-17Rα; TGFβRII; CCR6; and CX₃CR1, each of which displays differential expression on TAM versus ATM.

The invention also provides methods of modulating TAM and/or ATM by specifically inhibiting TAM and/or ATM function. For example, TAM and/or ATM may mediate certain of its effects and activities through secretion of one or more cellular messengers, such as cytokines or chemokines (i.e., TAM-mediated induction of certain T regulatory cells or inflammatory T cells requiring TGFβ activity, as shown herein). Specifically inhibiting or blocking secretion of, and/or removing from the environment one or more such cellular messengers normally secreted by TAM and/or ATM can have the effect of blocking TAM and/or ATM effects and activities. Such inhibition can be by, for example, administering a TAM and/or ATM cytokine/chemokine binding agent (including, but not limited to, an anti-cellular messenger antibody or fragment thereof (such as an anti-TGFβ antibody), and a small molecule). Chemokines and cytokines expressed by TAM or ATM include, but are not limited to, the cytokines and chemokines shown in FIGS. 14A and 14C.

The invention also provides methods for selectively producing and/or isolating certain immune cells. As shown herein, TAM and ATM are both specialized immune cells with certain properties of both macrophages and dendritic cells. TAM represent a small portion of the immune infiltrate in tumors, and have been difficult to obtain. The methods of the invention for isolating TAM based on their expression of both certain dendritic cell and certain macrophage cell surface markers offer a useful way to obtain TAM from mixed cell populations for use in research or therapeutically. Similarly, the methods of the invention for isolating ATM based on their expression of both certain dendritic cell and certain macrophage cell surface markers offer a useful way to obtain ATM from mixed cell populations for use in research or therapeutically. It will be appreciated by one of ordinary skill in the art that TAM and ATM may be separately isolated or purified by basing the isolation or purification on a combination of cell surface markers that differ between the cell types. As one nonlimiting example, TAM express IL-4Rα, while ATM do not. Other examples include, but are not limited to, those cytokine receptors and chemokine receptors that are differentially expressed in TAM and ATM (see FIGS. 14B and 14E). The invention also provides methods of selectively inducing IL-10⁺CD4⁺ Trl cells, FoxP3⁺CD4⁺ T regulatory cells, and/or TH₁₇ cells from nayve T cell cultures by stimulating the cultures with TAM or ATM. Being able to reproducibly produce these three T cell types in quantity is useful therapeutically and for research.

Compositions comprising one or more of the agents described above (i.e., IRTM-targeting agents (i.e., TAM-targeting agents or ATM-targeting agents) and/or IRTM cellular messenger-targeting agents (i.e., TAM cellular messenger-targeting agents or ATM cellular messenger-targeting agents) are provided. The invention also provides combination treatment methods and compositions that incorporate not only one or more agents specifically targeting IRTM (i.e., TAM- or ATM-targeting agents and/or cellular messengers secreted by TAM or ATM) but also one or more chemotherapeutic agent, cytokine, chemokine, anti-angiogenic agent, immunosuppressive agent, cytotoxic agent, or growth inhibitory agent. These combination treatments can suppress tumor angiogenesis and growth and/or treat inflammatory or autoimmune disorders. Combination treatments may be administered simultaneously or sequentially.

Additionally, kits are provided. Such kits may include one or more composition or combination treatment described herein, and may additionally include means for measuring and/or administering an appropriate dosage to a subject in need of such treatment and optionally further contain instructions for use.

### Diagnostics

The invention also provides for methods and compositions for diagnosing cell proliferative disorders, angiogenic disorders, and inflammatory, angiogenic and immunologic disorders (including, but not limited to, autoimmune disorders). In certain embodiments of the invention, methods of the invention compare the levels of TAM or ATM present in a test and reference cell population. The information disclosed herein regarding cell surface markers of TAM and ATM that differentiate TAM and/or ATM from both macrophages and dendritic cells, combined with protein and nucleic acid detection systems known in the art, allow for detection of the presence of and comparison of the relative amounts present in different cell populations/samples.

The test cell population can be any number of cells, i.e., one or more cells, and can be provided in vitro, in vivo, or ex vivo. In certain embodiments, cells in the reference cell population are derived from a tissue type as similar as possible to that of the test sample, e.g., tumor cell population. In some embodiments, the reference cell population is derived from the same subject as the test cell population, e.g., from a region proximal to the region of origin of the test cell population. In some embodiments, the reference cell population is derived from the same tissue type as the test cell population, but was collected from the subject at a different time (e.g., from a time earlier than the test cell population). In some embodiments, a series of reference cell population samples are collected at regular time intervals from the subject (e.g., daily, weekly, monthly, or yearly). In one embodiment of the invention, the reference cell population is derived from a plurality of cells. For example, the reference cell population can be a database of TAM and/or ATM expression patterns from previously tested cells.

### Protein and Nucleic Acid Detection Methods

Detecting the presence, activity, or amount of a protein of the invention can be readily performed using methods known in the art. Expression can be measured at the protein level, i.e., by measuring the levels of polypeptides. Such methods are well known in the art and include, e.g., immunoassays based on antibodies to the proteins. Expression levels of one or more of the protein sequences in the test cell population can be compared to expression levels of the sequences in one or more cells from a reference cell population. Expression of sequences in test and control populations of cells can be compared using any art-recognized method for comparing expression of nucleic acid sequences. For example, expression can be compared using GENECALLING™ methods as described in U.S. Pat. No. 5,871,697 and in Shimkets et al., Nat. Biotechnol. 17:798-803. In certain embodiments of the invention, expression of one, two or more, three or more, four or more, five or more, six or more, seven or more, eight or more, nine or more, ten or more, eleven or more, twelve or more, thirteen or more, fourteen or more, fifteen or more, 20 or more, 25 or more protein sequences are measured.

Various assay techniques known in the art may also be employed, such as competitive binding assays, direct or indirect sandwich assays and immunoprecipitation assays conducted in either heterogeneous or homogeneous phases (Zola, Monoclonal Antibodies: A Manual of Techniques, CRC Press, Inc. (1987) pp. 147-158). Antibodies or antigen-binding fragments thereof used in the assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. And Cytochem., 30:407 (1982).

Nucleic acid detection techniques are also well known in the art, and may be employed, in one embodiment, to assess the presence of mRNA for one or more TAM and/or ATM cell surface marker or other TAM and/or ATM-specific molecule and thus to determine the presence or amount of TAM and/or ATM in a cell population from which the cell sample was drawn. In certain embodiments, the presence or amount of mRNA encoding at least two different TAM and/or ATM cell surface markers is assessed. Methods commonly known in the art of recombinant DNA technology which can be used to assess the presence, amount, or activity of nucleic acids are described, e.g., in Ausubel et al. eds. (1993) Current Protocols in Molecular Biology, John Wiley & Sons, NY; and Kriegler (1990) Gene Transfer and Expression, A Laboratory Manual, Stockton Press, NY.

Optionally, comparison of differentially expressed sequences between a test cell population and a reference cell population can be done with respect to a control nucleic acid whose expression is independent of the parameter or condition being measured. Expression levels of the control nucleic acid in the test and reference nucleic acid can be used to normalize signal levels in the compared populations. Suitable control nucleic acids can readily be determined by one of ordinary skill in the art.

### Diagnostic or Marker Sets

The invention also provides for marker sets to identify TAM and/or ATM. In certain embodiments, these marker sets are provided in a kit for assessing the presence of TAM and/or ATM. For example, a marker set can include two or more, three or more, four or more, five or more, six or more, seven or more, eight or more, nine or more, ten or more, twelve or more, thirteen or more, fourteen or more, fifteen or more, twenty or more, or the entire set, of molecules. The molecule is a nucleic acid encoding an intracellular protein, a secreted protein, or a cell surface marker of TAM and/or ATM, and includes, but is not limited to, F4/80, CD11c, and langerin. In one embodiment of the invention, an antibody is provided that detects one or more such protein. As shown herein, TAM and ATM express cell surface markers of both macrophages and dendritic cells, and thus marker sets to detect TAM and/or ATM may contain both macrophage markers and dendritic cell markers. It will be recognized that a dendritic cell marker alone can be used to detect TAM, ATM, and dendritic cells generally, and that a macrophage marker alone can be used to detect TAM, ATM, and macrophages generally.

### Therapeutic Uses

It is contemplated that, according to the invention, the combinations of modulators, including TAM and/or ATM agonists, TAM and/or ATM antagonists, TAM-binding agents, ATM-binding agents, agonists of TAM-secreted cytokines/chemokines, agonists of ATM-secreted cytokines, antagonists of TAM-secreted cytokines/chemokines, antagonists of TAM-secreted cytokines/chemokines, TAM-secreted cytokines/chemokines binding agents, and ATM-secreted cytokines/chemokines binding agents, alone or in combination with one another or with other therapeutic agents (including, but not limited to, a chemotherapeutic agent, a cytokine, a chemokine, an anti-angiogenic agent, an immunosuppressive agent, a cytotoxic agent, and a growth inhibitory agent) can be used to treat various conditions such as cell proliferative disorders, angiogenic disorders, and inflammatory, angiogenic and immunologic disorders (including, but not limited to, autoimmune disorders). In one embodiment, modulators of TAM viability, presence, or activity are used in the inhibition of cancer cell or tumor growth. In certain embodiments of the invention, TAM-binding agents, TAM antagonists, antagonists of TAM-secreted cytokines/chemokines and/or TAM-secreted cytokines/chemokines binding agents are used to treat a proliferative disorder, for example, to inhibit cancer cell or tumor growth, or to inhibit metastasis of a tumor. See also section entitled Combination Therapies herein. Examples of neoplastic disorders to be treated include, but are not limited to, those described herein under the terms “cancer” and “cancerous.” In another embodiment, modulators of TAM viability, presence, or activity are used in the treatment of immune disorders, including, but not limited to, autoimmune disorders. In certain embodiments of the invention, TAM agonists, TAM-binding agents, agonists of TAM-secreted cytokines/chemokines, and/or TAM-secreted cytokines/chemokines binding agents are used to stimulate TAM presence, growth and/or activity are used to treat autoimmune disorders, e.g., by stimulating TAM-induced growth and differentiation of to IL-10⁺ CD4⁺ Trl cells and FoxP3⁺ CD4⁺ T regulatory cells from naïve T cell populations. Examples of autoimmune disorders to be treated include, but are not limited to, those described herein under the term “autoimmune disorder”. In another embodiment, modulators of ATM viability, presence, or activity are used in the inhibition of inflammatory disorders, including, but not limited to, hyperglycemic disorders and insulin resistance disorders. In certain embodiments of the invention, ATM-binding agents, ATM antagonists, antagonists of ATM-secreted cytokines/chemokines and/or ATM-secreted cytokines/chemokines binding agents are used to inhibit inflammatory disorders, including, but not limited to, hyperglycemic disorders and insulin resistance disorders.

### Combination Therapies

As indicated above, the invention provides combined therapies in which a TAM binding agent, an ATM binding agent, a TAM agonist, an ATM agonist, a TAM antagonist, an ATM antagonist, a TAM-secreted cytokine/chemokine binding agent, an ATM-secreted cytokine/chemokine binding agent, an agonist of a TAM-secreted cytokine/chemokine, an agonist of an ATM-secreted cytokine/chemokine, an antagonist of a TAM-secreted cytokine/chemokine, or an antagonist of an ATM-secreted cytokine/chemokine is administered in combination with another therapy. For example, a TAM binding agent can be administered in combination with a different agent, agonist or antagonist of the invention to treat, e.g., a proliferative disorder or an autoimmune disorder. As another example, an ATM binding agent can be administered in combination with a different agent, agonist or antagonist of the invention to treat, e.g., an inflammatory disorder including, but not limited to, a hyperglycemic disorder or an insulin resistance disorder. In certain embodiments, additional agents, e.g. a chemotherapeutic agent, a cytokine, a chemokine, an anti-angiogenic agent, an immunosuppressive agent, a cytotoxic agent, an antiinflammatory, and a growth inhibitory agent may be employed. The agents, agonists and antagonists of the invention can be administered serially or in combination with another agent that is effective for those purposes, either in the same composition or as separate compositions. Alternatively, or additionally, multiple antagonists, agents and/or agonists of the invention can be administered.

The administration of the agonist, antagonist and/or agents of the invention can be done simultaneously, e.g., as a single composition or as two or more distinct compositions using the same or different administration routes. Alternatively, or additionally, the administration can be done sequentially, in any order. In certain embodiments, intervals ranging from minutes to days, to weeks to months, can be present between the administrations of the two or more compositions. However, simultaneous administration or administration of the different agonist, antagonist or agent of the invention first is also contemplated.

The effective amounts of therapeutic agents administered in combination with an agonist, antagonist or agent of the invention will be at the physicians' or veterinarian's discretion. Dosage administration and adjustment is done to achieve maximal management of the conditions to be treated. The dose will additionally depend on such factors as the type of therapeutic agent to be used and the specific patient being treated. In certain embodiments, the combination of several like molecules (e.g., several antagonists) potentiates the efficacy of a single molecule. The term “potentiate” refers to an improvement in the efficacy of a therapeutic agent at its common or approved dose. See also the section entitled Pharmaceutical Compositions herein.

In certain aspects of the invention, other therapeutic agents useful for combination tumor therapy with TAM and/or ATM binding agents, TAM and/or ATM antagonists, agonists of TAM and/or ATM-secreted cytokine/chemokines and TAM and/or ATM-secreted binding agents of the invention include other cancer therapies, (e.g., surgery, radiological treatments (e.g., involving irradiation or administration of radioactive substances), chemotherapy, treatment with anti-cancer agents listed herein and known in the art, or combinations thereof). Alternatively, or additionally, two or more antibodies binding the same or two or more different antigens disclosed herein can be co-administered to the patient. Sometimes, it may be beneficial to also administer one or more cytokines to the patient.

### Chemotherapeutic Agents

In certain aspects, the invention provides a method of blocking or reducing tumor growth or growth of a cancer cell, by administering effective amounts of a TAM antagonist, a TAM binding agent, an antagonist of a TAM-secreted cytokine/chemokine and/or a TAM-secreted cytokine/chemokine binding agent of the invention and one or more chemotherapeutic agents to a patient susceptible to, or diagnosed with, cancer. A variety of chemotherapeutic agents may be used in the combined treatment methods of the invention. An exemplary and non-limiting list of chemotherapeutic agents contemplated is provided herein under the term “chemotherapeutic agent”.

As will be understood by those of ordinary skill in the art, the appropriate doses of chemotherapeutic agents will be generally around those already employed in clinical therapies wherein the chemotherapeutics are administered alone or in combination with other chemotherapeutics. Variation in dosage will likely occur depending on the condition being treated. The physician administering treatment will be able to determine the appropriate dose for the individual subject.

### Antibodies

Antibodies of the invention include antibodies the specifically binds to a protein of the invention and antibody fragment of such antibodies. A polypeptide or protein of the invention includes, but not limited to, a TAM cell surface marker (including, but not limited to, F4/80, CD11c, and, e.g., the cytokine and chemokine receptors expressed by TAM set forth in FIGS. 14B and 14E) and a TAM cytokine or chemokine (including, but not limited to, TGFβ and, e.g., the cytokines and chemokines expressed by TAM set forth in FIGS. 14A and 14C). In certain aspects, a polypeptide or protein of the invention is an antibody that specifically binds to a TAM cell surface marker (including, but not limited to, F4/80, CD11c, and, e.g., the cytokine and chemokine receptors expressed by TAM set forth in FIGS. 14B and 14E) and a TAM cytokine or chemokine (including, but not limited to, TGFβ and, e.g., the cytokines and chemokines expressed by TAM set forth in FIGS. 14A and 14C).

Antibodies of the invention further include antibodies that are anti-angiogenesis agents or angiogenesis inhibitors, antibodies that are myeloid cell reduction agents, antibodies that are anti-cancer agents, or other antibodies described herein. Exemplary antibodies include, e.g., polyclonal, monoclonal, humanized, fragment, bispecific, multispecific, heteroconjugated, multivalent, effector function-containing, etc., antibodies.

### Polyclonal Antibodies

The antibodies of the invention can comprise polyclonal antibodies. Methods of preparing polyclonal antibodies are known to the skilled artisan. For example, polyclonal antibodies against an antibody of the invention are raised in animals by one or multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

In one embodiment, animals are immunized against a molecule of the invention, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Typically, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

### Monoclonal Antibodies

Monoclonal antibodies against an antigen described herein can be made using the hybridoma method first described by Kohler et al., Nature, 256:495 (1975), or may be made by recombinant DNA methods (see, e.g., U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that typically contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Typical myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the target of interest. The binding specificity of monoclonal antibodies produced by hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography. The monoclonal antibodies may also be made by recombinant DNA methods, such as those described in U.S. Pat. No. 4,816,567. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In another embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

### Humanized and Human Antibodies

Antibodies of the invention can comprise humanized antibodies or human antibodies. A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a typical method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992). Human antibodies can also be derived from phage-display libraries (Hoogenboom et al., J. Mol. Biol., 227:381 (1991); Marks et al., J. Mol. Biol., 222:581-597 (1991); Vaughan et al. Nature Biotech 14:309 (1996)).

Human antibodies can also be produced using various techniques known in the art, including phage display libraries (Hoogenboom and Winter, J. Mol. Biol., 227:381 (1991); Marks et al., J. Mol. Biol., 222:581 (1991)). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, K S. and Chiswell, D J., Cur Opin in Struct Biol 3:564-571 (1993). Several sources of V-gene segments can be used for phage display. For example, Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated, e.g., by essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)). Human antibodies may also be generated by in vitro activated B cells (see U.S. Pat. Nos. 5,567,610 and 5,229,275).

### Antibody Fragments

Antibody fragments are also included in the invention. Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. Other techniques for the production of antibody fragments will be apparent to one of ordinary skill in the art. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. SFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

### Multispecific Antibodies (e.g., Bispecific)

Antibodies of the invention also include, e.g., multispecific antibodies, which have binding specificities for at least two different antigens. While such molecules normally will only bind two antigens (i.e. bispecific antibodies, BsAbs), antibodies with additional specificities such as trispecific or other multispecific (i.e., four or more specificities encompassed in one molecule) antibodies are encompassed by this expression when used herein. Examples of BsAbs known in the art include those with one arm directed against a tumor cell antigen and the other arm directed against a cytotoxic trigger molecule such as anti-FcγRI/anti-CD15, anti-p185^{HER2}/FcγRIII (CD16), anti-CD3/anti-malignant B-cell (1D10), anti-CD3/anti-p185^{HER2}, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell adhesion molecule (NCAM)/anti-CD3, anti-folate binding protein (FBP)/anti-CD3, anti-pan carcinoma associated antigen (AMOC-31)/anti-CD3; BsAbs with one arm which binds specifically to a tumor antigen and one arm which binds to a toxin such as anti-saporin/anti-Id-1, anti-CD22/anti-saporin, anti-CD7/anti-saporin, anti-CD38/anti-saporin, anti-CEA/anti-ricin A chain, anti-interferon-α (IFN-α)/anti-hybridoma idiotype, anti-CEA/anti-vinca alkaloid; BsAbs for converting enzyme activated prodrugs such as anti-CD30/anti-alkaline phosphatase (which catalyzes conversion of mitomycin phosphate prodrug to mitomycin alcohol); BsAbs which can be used as fibrinolytic agents such as anti-fibrin/anti-tissue plasminogen activator (tPA), anti-fibrin/anti-urokinase-type plasminogen activator (uPA); BsAbs for targeting immune complexes to cell surface receptors such as anti-low density lipoprotein (LDL)/anti-Fc receptor (e.g. FcγRI, FcγRII or FcγRIII); BsAbs for use in therapy of infectious diseases such as anti-CD3/anti-herpes simplex virus (HSV), anti-T-cell receptor: CD3 complex/anti-influenza, anti-FcγR/anti-HIV; BsAbs for tumor detection in vitro or in vivo such as anti-CEA/anti-EOTUBE, anti-CEA/anti-DPTA, anti-p185^{HER2}/anti-hapten; BsAbs as vaccine adjuvants; and BsAbs as diagnostic tools such as anti-rabbit IgG/anti-ferritin, anti-horse radish peroxidase (HRP)/anti-hormone, anti-somatostatin/anti-substance P, anti-HRP/anti-FITC, anti-CEA/anti-β-galactosidase. Examples of trispecific antibodies include anti-CD3/anti-CD4/anti-CD37, anti-CD3/anti-CD5/anti-CD37 and anti-CD3/anti-CD8/anti-CD37. In certain aspects of the invention one of the antibodies in the bispecific antibody can be coupled to a macrophage-specific cellular marker and the other to a dendritic cell-specific cellular marker. In certain embodiments, such an antibody would bind more tightly to a cell bearing both the given macrophage-specific cellular marker and the given dendritic cell-specific cellular marker than to a cell bearing only one or the other marker.

Bispecific antibodies can be prepared as full length antibodies or antibody fragments (e.g. F(ab′)₂ bispecific antibodies). Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described, e.g., in WO96/27011, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Techniques for generating bispecific antibodies from antibody fragments are also known in the art. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the VEGF receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are also contemplated. For example, trispecific antibodies can be prepared. Tutt et al. J. Immunol. 147: 60 (1991).

### Heteroconjugate Antibodies

Bispecific antibodies include cross-linked or “heteroconjugate” antibodies, which are antibodies of the invention. Such bispecific antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in, e.g., U.S. Pat. No. 4,676,980, along with a number of cross-linking techniques.

### Multivalent Antibodies

Antibodies of the invention include a multivalent antibody. A multivalent antibody may be internalized (and/or catabolized) faster than a bivalent antibody by a cell expressing an antigen to which the antibodies bind. The antibodies of the invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain. Multivalent antibodies may have multiple binding sites for the same antigen, or binding sites for two or more different antigens.

### Effector Function Engineering

It may be desirable to modify the antibody of the invention with respect to effector function, so as to enhance the effectiveness of the antibody in treating a particular disorder or disease. For example, a cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

### Immunoconjugates

The invention also pertains to immunoconjugates comprising an antibody described herein conjugated to a cytotoxic agent such as a chemotherapeutic agent, toxin (e.g. an enzymatically active toxin of bacterial, fungal, plant or animal origin, or fragments thereof), or a radioactive isotope (i.e., a radioconjugate). A variety of radionuclides are available for the production of radioconjugate antibodies. Examples include, but are not limited to, e.g., ²¹²Bi, ¹³¹I, ¹³¹In, ⁹⁰Y and ¹⁸⁶Re.

Chemotherapeutic agents useful in the generation of such immunoconjugates have been described above. For example, BCNU, streptozoicin, vincristine, 5-fluorouracil, the family of agents known collectively LL-E33288 complex described in U.S. Pat. Nos. 5,053,394, 5,770,710, esperamicins (U.S. Pat. No. 5,877,296), etc. (see also the definition of chemotherapeutic agents herein) can be conjugated to antibodies of the invention or fragments thereof.

For selective destruction of the tumor, the antibody may comprise a highly radioactive atom. A variety of radioactive isotopes are available for the production of radioconjugated antibodies or fragments thereof. Examples include, but are not limited to, e.g., ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb, ¹¹¹In, radioactive isotopes of Lu, etc. When the conjugate is used for diagnosis, it may comprise a radioactive atom for scintigraphic studies, for example ^{99m}tc or ¹²³I, or a spin label for nuclear magnetic resonance (NMR) imaging (also known as magnetic resonance imaging, MRI), such as iodine-123, iodine-131, indium-111, fluorine-19, carbon-13, nitrogen-15, oxygen-17, gadolinium, manganese or iron. The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as ^{99m}tc or ¹²³I, ¹⁸⁶Re, ¹⁸⁸Re and ¹¹¹In can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. See, e.g., Monoclonal Antibodies in Immunoscintigraphy (Chatal, CRC Press 1989) which describes other methods in detail.

Enzymatically active toxins and fragments thereof which can be used include diphtheria A chain, nonbinding active fragments of diphtheria toxin, anthrax toxin protective antigen, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), momordica charantia inhibitor, curcin, crotin, sapaonaria officinalis inhibitor, gelonin, mitogellin, restrictocin, phenomycin, neomycin, and the tricothecenes. See, e.g., WO 93/21232 published Oct. 28, 1993.

Conjugates of the antibody and cytotoxic agent can be made using a variety of bifunctional protein coupling agents such as N-succinimidyl-3-(2-pyridyldithiol) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

Alternatively, a fusion protein comprising the anti-VEGF, and/or the anti-protein of the invention antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In certain embodiments, the antibody is conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionucleotide). In certain embodiments, an immunoconjugate is formed between an antibody and a compound with nucleolytic activity (e.g., a ribonuclease or a DNA endonuclease such as a deoxyribonuclease; Dnase).

### Maytansine and Maytansinoids

The invention further provides an antibody of the invention conjugated to one or more maytansinoid molecules. Maytansinoids are mitotic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

An antibody of the invention can be conjugated to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. In one embodiment, maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1, and Chari et al., Cancer Research 52:127-131 (1992). The linking groups include disulfide groups, thioether groups, acid labile groups, photolabile groups, peptidase labile groups, or esterase labile groups, as disclosed in the above-identified patents, disulfide and thioether groups being preferred.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Typical coupling agents include N-succinimidyl-3-(2-pyridyldithio) propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. The linkage is formed at the C-3 position of maytansinol or a maytansinol analogue.

### Calicheamicin

Another immunoconjugate of interest comprises an antibody of the invention conjugated to one or more calicheamicin molecules. The calicheamicin family of antibiotics is capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ_{I}¹ (Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

### Other Antibody Modifications

Other modifications of an antibody of the invention are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules, or in macroemulsions. Such techniques are disclosed in Remington's Pharmaceutical Sciences, 16th edition, Oslo, A., Ed., (1980).

### Liposomes and Nanoparticles

Polypeptides of the invention can be formulated in liposomes. For example, antibodies of the invention can be formulated as immunoliposomes. Liposomes containing the antibody are prepared by methods known in the art, such as described in Epstein et al., Proc. Natl. Acad. Sci. USA, 82:3688 (1985); Hwang et al., Proc. Natl. Acad. Sci. USA, 77:4030 (1980); and U.S. Pat. Nos. 4,485,045 and 4,544,545. Liposomes with enhanced circulation time are disclosed in U.S. Pat. No. 5,013,556. Generally, the formulation and use of liposomes is known to those of skill in the art.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the invention can be conjugated to the liposomes as described in Martin et al. J. Biol. Chem. 257: 286-288 (1982) via a disulfide interchange reaction. A chemotherapeutic agent (such as Doxorubicin) is optionally contained within the liposome. See Gabizon et al. J. National Cancer Inst. 81(19)1484 (1989).

### Covalent Modifications to Polypeptides of the Invention

Covalent modifications of a polypeptide of the invention, e.g., a protein of the invention, an antibody of a protein of the invention, a polypeptide antagonist or agonist fragment, a fusion molecule (e.g., an immunofusion molecule), etc., are included within the scope of this invention. They may be made by chemical synthesis or by enzymatic or chemical cleavage of the polypeptide, if applicable. Other types of covalent modifications of the polypeptide are introduced into the molecule by reacting targeted amino acid residues of the polypeptide with an organic derivatizing agent that is capable of reacting with selected side chains or the N- or C-terminal residues, or by incorporating a modified amino acid or unnatural amino acid into the growing polypeptide chain, e.g., Ellman et al. Meth. Enzym. 202:301-336 (1991); Noren et al. Science 244:182 (1989); and, & US Patent application publications 20030108885 and 20030082575.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethyl-pyro-carbonate at pH 5.5-7.0 because this agent is relatively specific for the histidyl side chain. Para-bromophenacyl bromide also is useful; the reaction is typically performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidazole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification involves chemically or enzymatically coupling glycosides to a polypeptide of the invention. These procedures are advantageous in that they do not require production of the polypeptide in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published 11 Sep. 1987, and in Aplin and Wriston, CRC Crit. Rev. Biochem., pp. 259-306 (1981). Removal of any carbohydrate moieties present on a polypeptide of the invention may be accomplished chemically or enzymatically. Chemical deglycosylation requires exposure of the polypeptide to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin, et al. Arch. Biochem. Biophys. 259:52 (1987) and by Edge et al. Anal. Biochem., 118:131 (1981). Enzymatic cleavage of carbohydrate moieties, e.g., on antibodies, can be achieved by the use of a variety of endo- and exo-glycosidases as described by Thotakura et al. Meth. Enzymol. 138:350 (1987).

Another type of covalent modification of a polypeptide of the invention comprises linking the polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

### Vectors, Host Cells and Recombinant Methods

The polypeptides of the invention can be produced recombinantly, using techniques and materials readily obtainable.

For recombinant production of a polypeptide of the invention, e.g., a protein of the invention, e.g., an antibody of the invention, the nucleic acid encoding it is isolated and inserted into a replicable vector for further cloning (amplification of the DNA) or for expression. DNA encoding the polypeptide of the invention is readily isolated and sequenced using conventional procedures. For example, a DNA encoding a monoclonal antibody is isolated and sequenced, e.g., by using oligonucleotide probes that are capable of binding specifically to genes encoding the heavy and light chains of the antibody. Many vectors are available. The vector components generally include, but are not limited to, one or more of the following: a signal sequence, an origin of replication, one or more marker genes, an enhancer element, a promoter, and a transcription termination sequence.

### Signal Sequence Component

Polypeptides of the invention may be produced recombinantly not only directly, but also as a fusion polypeptide with a heterologous polypeptide, which is typically a signal sequence or other polypeptide having a specific cleavage site at the N-terminus of the mature protein or polypeptide. The heterologous signal sequence selected typically is one that is recognized and processed (i.e., cleaved by a signal peptidase) by the host cell. For prokaryotic host cells that do not recognize and process the native polypeptide signal sequence, the signal sequence is substituted by a prokaryotic signal sequence selected, for example, from the group of the alkaline phosphatase, penicillinase, lpp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available. The DNA for such precursor region is ligated in reading frame to DNA encoding the polypeptide of the invention.

### Origin of Replication Component

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Selection Gene Component Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, typically primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity.

Alternatively, host cells (particularly wild-type hosts that contain endogenous DHFR) transformed or co-transformed with DNA sequences encoding a polypeptide of the invention, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid Yrp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

### Promoter Component

Expression and cloning vectors usually contain a promoter that is recognized by the host organism and is operably linked to a nucleic acid encoding a polypeptide of the invention. Promoters suitable for use with prokaryotic hosts include the phoA promoter, β-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the polypeptide of the invention.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Transcription of polypeptides of the invention from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and typically Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the rous sarcoma virus long terminal repeat can be used as the promoter.

### Enhancer Element Component

Transcription of a DNA encoding a polypeptide of this invention by higher eukaryotes is often increased by inserting an enhancer sequence into the vector. Many enhancer sequences are now known from mammalian genes (globin, elastase, albumin, α-fetoprotein, and insulin). Typically, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide-encoding sequence, but is typically located at a site 5′ from the promoter.

### Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the polypeptide of the invention. One useful transcription termination component is the bovine growth hormone polyadenylation region. See WO94/11026 and the expression vector disclosed therein.

### Selection and Transformation of Host Cells

Suitable host cells for cloning or expressing DNA encoding the polypeptides of the invention in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. Typically, the E. coli cloning host is E. coli 294 (ATCC 31,446), although other strains such as E. coli B, E. coli X11776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for polypeptide of the invention-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated polypeptides of the invention are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2). Host cells are transformed with the above-described expression or cloning vectors for polypeptide of the invention production and cultured in conventional nutrient media modified as appropriate for inducing promoters, selecting transformants, or amplifying the genes encoding the desired sequences.

### Culturing the Host Cells

The host cells used to produce polypeptides of the invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Polypeptide Purification

A polypeptide or protein of the invention may be purified. When using recombinant techniques, a polypeptide of the invention can be produced intracellularly, in the periplasmic space, or directly secreted into the medium. Polypeptides of the invention may be recovered from culture medium or from host cell lysates. If membrane-bound, it can be released from the membrane using a suitable detergent solution (e.g. Triton-X 100) or by enzymatic cleavage. Cells employed in expression of a polypeptide of the invention can be disrupted by various physical or chemical means, such as freeze-thaw cycling, sonication, mechanical disruption, or cell lysing agents.

The following procedures are exemplary of suitable protein purification procedures: by fractionation on an ion-exchange column; ethanol precipitation; reverse phase HPLC; chromatography on silica, chromatography on heparin SEPHAROSE™ chromatography on an anion or cation exchange resin (such as a polyaspartic acid column, DEAE, etc.); chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of polypeptides of the invention. Various methods of protein purification may be employed and such methods are known in the art and described for example in Deutscher, Methods in Enzymology, 182 (1990); Scopes, Protein Purification Principles and Practice, Springer-Verlag, New York (1982). The purification step(s) selected will depend, for example, on the nature of the production process used and the particular polypeptide of the invention produced.

For example, an antibody composition prepared from the cells can be purified using, for example, hydroxylapatite chromatography, gel electrophoresis, dialysis, and affinity chromatography, with affinity chromatography being the typical purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification, e.g., those indicated above, are also available depending on the antibody to be recovered. See also, Carter et al., Bio/Technology 10:163-167 (1992) which describes a procedure for isolating antibodies which are secreted to the periplasmic space of E. coli.

### Pharmaceutical Compositions

Therapeutic formulations of agents of the invention (e.g., TAM and/or ATM-binding agents or TAM and/or ATM-secreted cellular messenger-binding agents), and combinations thereof as described herein used in accordance with the invention are prepared for storage by mixing a molecule, e.g., polypeptide(s), having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by, e.g., filtration through sterile filtration membranes.

Sustained-release preparations may be prepared. Suitable examples of sustained-release preparations include semipermeable matrices of solid hydrophobic polymers containing a polypeptide of the invention, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions. See also, e.g., U.S. Pat. No. 6,699,501, describing capsules with polyelectrolyte covering.

It is further contemplated that an agent of the invention (e.g., TAM agonist, TAM antagonist, or an agonist or antagonist of TAM cytokine/chemokine secretion) can be introduced to a subject by gene therapy. Gene therapy refers to therapy performed by the administration of a nucleic acid to a subject. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 (1986)). The oligonucleotides can be modified to enhance their uptake, e.g. by substituting their negatively charged phosphodiester groups by uncharged groups. For general reviews of the methods of gene therapy, see, for example, Goldspiel et al. Clinical Pharmacy 12:488-505 (1993); Wu and Wu Biotherapy 3:87-95 (1991); Tolstoshev Ann. Rev. Pharmacol. Toxicol. 32:573-596 (1993); Mulligan Science 260:926-932 (1993); Morgan and Anderson Ann. Rev. Biochem. 62:191-217 (1993); and May TIBTECH 11:155-215 (1993). Methods commonly known in the art of recombinant DNA technology which can be used are described in Ausubel et al. eds. (1993) Current Protocols in Molecular Biology, John Wiley & Sons, NY; and Kriegler (1990) Gene Transfer and Expression, A Laboratory Manual, Stockton Press, NY.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 (1993)). For example, in vivo nucleic acid transfer techniques include transfection with viral vectors (such as adenovirus, Herpes simplex I virus, lentivirus, retrovirus, or adeno-associated virus) and lipid-based systems (useful lipids for lipid-mediated transfer of the gene are DOTMA, DOPE and DC-Chol, for example). Examples of using viral vectors in gene therapy can be found in Clowes et al. J. Clin. Invest. 93:644-651 (1994); Kiem et al. Blood 83:1467-1473 (1994); Salmons and Gunzberg Human Gene Therapy 4:129-141 (1993); Grossman and Wilson Curr. Opin. in Genetics and Devel. 3:110-114 (1993); Bout et al. Human Gene Therapy 5:3-10 (1994); Rosenfeld et al. Science 252:431-434 (1991); Rosenfeld et al. Cell 68:143-155 (1992); Mastrangeli et al. J. Clin. Invest. 91:225-234 (1993); and Walsh et al. Proc. Soc. Exp. Biol. Med. 204:289-300 (1993).

In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

### Dosage and Administration

The agents of the invention (TAM and/or ATM binding agent, TAM and/or ATM agonist, TAM and/or ATM antagonist, TAM and/or ATM-secreted cytokine/chemokine binding agent, agonist of TAM and/or ATM-secreted cytokine/chemokine, and/or antagonist of TAM and/or ATM-secreted cytokine/chemokine) are administered to a mammalian patient (i.e., a human patient), in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes, and/or subcutaneous administration.

In certain embodiments, the treatment of the invention involves the combined administration of a composition of the invention and one or more other therapeutic agent (e.g., a chemotherapeutic agent, a cytokine, a chemokine, an anti-angiogenic agent, an immunosuppressive agent, a cytotoxic agent, and a growth inhibitory agent). The invention also contemplates administration of multiple antibodies to the same antigen or multiple antibodies to different proteins of the invention. In one embodiment, a cocktail of different chemotherapeutic agents is administered with a composition of the invention. The combined administration includes coadministration, using separate formulations or a single pharmaceutical formulation, and/or consecutive administration in either order. In one embodiment, there is a time period while both (or all) active agents simultaneously exert their biological activities.

For the prevention or treatment of disease, the appropriate dosage of the agent of the invention will depend on the type of disease to be treated, as defined above, the severity and course of the disease, whether the inhibitor is administered for preventive or therapeutic purposes, previous therapy, the patient's clinical history and response to the inhibitor, and the discretion of the attending physician. The inhibitor is suitably administered to the patient at one time or over a series of treatments. In a combination therapy regimen, the compositions of the invention are administered in a therapeutically effective amount or a therapeutically synergistic amount. As used herein, a therapeutically effective amount is such that administration of a composition of the invention and/or co-administration of a composition of the invention and one or more other therapeutic agents, results in reduction or inhibition of the targeting disease or condition. The effect of the administration of a combination of agents can be additive. In one embodiment, the result of the administration is a synergistic effect. A therapeutically synergistic amount is that amount of a composition of the invention and one or more other therapeutic agents, e.g., a chemotherapeutic agent or an anti-cancer agent, necessary to synergistically or significantly reduce or eliminate conditions or symptoms associated with a particular disease.

Depending on the type and severity of the disease, about 1 μg/kg to 50 mg/kg (e.g. 0.1-20 mg/kg) of an agent, agonist or antagonist of the invention is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. Typically, the clinician will administered a molecule(s) of the invention until a dosage(s) is reached that provides the required biological effect. The progress of the therapy of the invention is easily monitored by conventional techniques and assays.

For example, preparation and dosing schedules for angiogenesis inhibitors, e.g., anti-VEGF antibodies, such as AVASTIN® (Genentech), may be used according to manufacturers' instructions or determined empirically by the skilled practitioner. In another example, preparation and dosing schedules for such chemotherapeutic agents may be used according to manufacturers' instructions or as determined empirically by the skilled practitioner. Preparation and dosing schedules for chemotherapy are also described in Chemotherapy Service Ed., M. C. Perry, Williams & Wilkins, Baltimore, Md. (1992).

### Efficacy of the Treatment

The efficacy of the treatment of the invention can be measured in some embodiments by various endpoints known in the art. In one embodiment, the efficacy of TAM-based treatments can be measured using various endpoints commonly used in evaluating neoplastic or non-neoplastic disorders. For example, cancer treatments can be evaluated by, e.g., but not limited to, tumor regression, tumor weight or size shrinkage, time to progression, duration of survival, progression free survival, overall response rate, duration of response, quality of life, protein expression and/or activity. Because the agents described herein target the tumor vasculature and infiltrate and not necessarily the neoplastic cells themselves, they represent a different class of anticancer drugs, and therefore can require different measures and definitions of clinical responses to drugs than standard anti-neoplastic cell therapies. For example, tumor shrinkage of greater than 50% in a 2-dimensional analysis is the standard cut-off for declaring a response. However, the inhibitors of the invention may cause inhibition of metastatic spread without shrinkage of the primary tumor, or may simply exert a tumouristatic effect. Accordingly, approaches to determining efficacy of the therapy can be employed, including for example, measurement of plasma or urinary markers of angiogenesis and measurement of response through radiological imaging.

In other embodiments, the efficacy of the treatment of the invention can be measured by various endpoints commonly used in evaluating autoimmune disorders. For example, autoimmune disorder treatments can be evaluated by methods including, but not limited to, diminishment or cessation of primary or secondary characteristics of the disease, time to progression, duration of survival, progression free survival, overall response rate, duration of response, quality of life, protein expression and/or activity. The same logic may be applied to measuring the efficacy of a treatment of the invention using endpoints commonly used by one of ordinary skill in the art for evaluating a particular disorder that the treatment of the invention is intended to address.

### Articles of Manufacture

In another embodiment of the invention, an article of manufacture containing materials useful for the treatment of the disorders or diagnosing the disorders described above is provided. The article of manufacture comprises a container, a label and a package insert. Suitable containers include, for example, bottles, vials, syringes, etc. The containers may be formed from a variety of materials such as glass or plastic. In one embodiment, the container holds a composition which is effective for treating the condition and may have a sterile access port (for example the container may be an intravenous solution bag or a vial having a stopper pierceable by a hypodermic injection needle). In one embodiment, at least one active agent in the composition is a TAM and/or ATM binding agent or a TAM and/or ATM-secreted cytokine/chemokine binding agent. In another embodiment, at least one active agent in the composition is a TAM and/or ATM agonist or an agonist of at least one TAM and/or ATM-secreted cytokine/chemokine. In another embodiment, at least one active agent in the composition is a TAM and/or ATM antagonist or an antagonist of at least one TAM and/or ATM-secreted cytokine/chemokine. In certain embodiments, the composition further includes at least a second active molecule including, but not limited to, a chemotherapeutic agent, a cytokine, a chemokine, an anti-angiogenic agent, an immunosuppressive agent, a cytotoxic agent, and a growth inhibitory agent. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. The articles of manufacture of the invention may further include other materials desirable from a commercial and user standpoint, including additional active agents, other buffers, diluents, filters, needles, and syringes.

It will be understood that the examples and embodiments described herein are for illustrative purposes only and that various modifications or changes in light thereof will be suggested to persons skilled in the art and are to be included within the spirit and purview of this application and scope of the appended claims. The specification is considered to be sufficient to enable one skilled in the art to practice the invention. All publications, patents, and patent applications cited herein are hereby incorporated by reference in their entirety for all purposes.

### EXAMPLES

### Example 1

### Composition and Localization of Myeloid Infiltrates

The composition and localization of immune infiltrate in MMTV-PyMT induced mammary tumors was assessed by immunohistochemistry. Wild-type mice sensitive to Friend leukemia virus B strain (“FVB”) were purchased (Charles River) and mice comprising MMTV.PyMT^{tg} or MMTV.Her2^{tg} tumors in an FVB background were bred in pathogen-free facilities. Tumors from MMTV.PyMT^{tg} mice were embedded in OCT solution and frozen. Frozen sections were cut into 5 micron slices, dried at room temperature, and fixed with ice cold acetone using standard procedures. Endogenous peroxidase was quenched with glucose oxidase for 60 minutes at 37° C. The sections were rinsed with PBS, and endogenous avidin and biotin blocked with an Avidin Biotin Blocking Kit (Vector) according to the manufacturer's instructions. The sections were blocked with 10% rabbit serum in 3% BSA/PBS for 30 minutes at room temperature, and then incubated with the appropriate antibody diluted in blocking serum for 60 minutes at room temperature, with rat IgG2b as a negative control. Sections were rinsed with TBST and incubated with an appropriate biotinylated secondary antibody for 30 minutes at room temperature. Sections were developed according to standard procedures. Rat anti-CD45 (LCA) antibody was obtained from Pharmingen, rabbit anti-CD3 antibody was obtained from DAKO, biotinylated goat anti-rabbit IgG and biotinylated rabbit anti-rat IgG were obtained from Vector, rat anti-F4/80 antibody was obtained from Serotec.

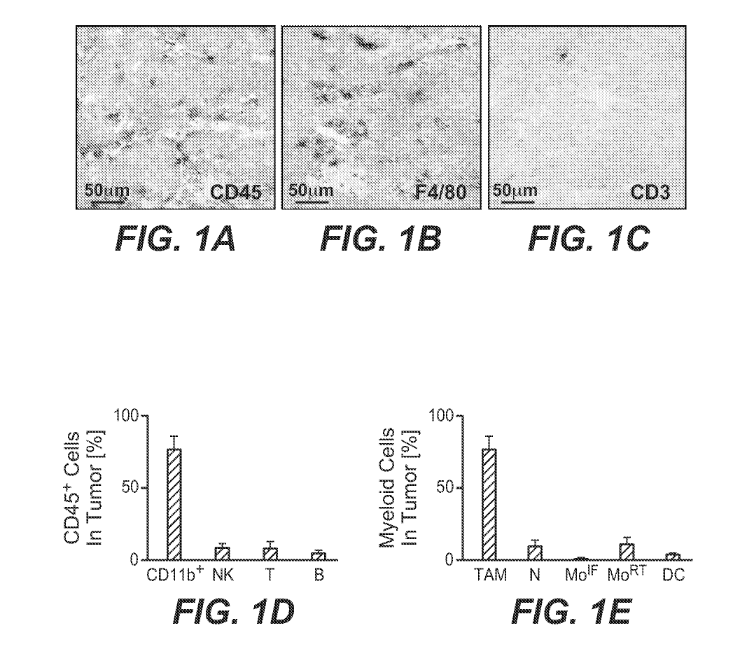

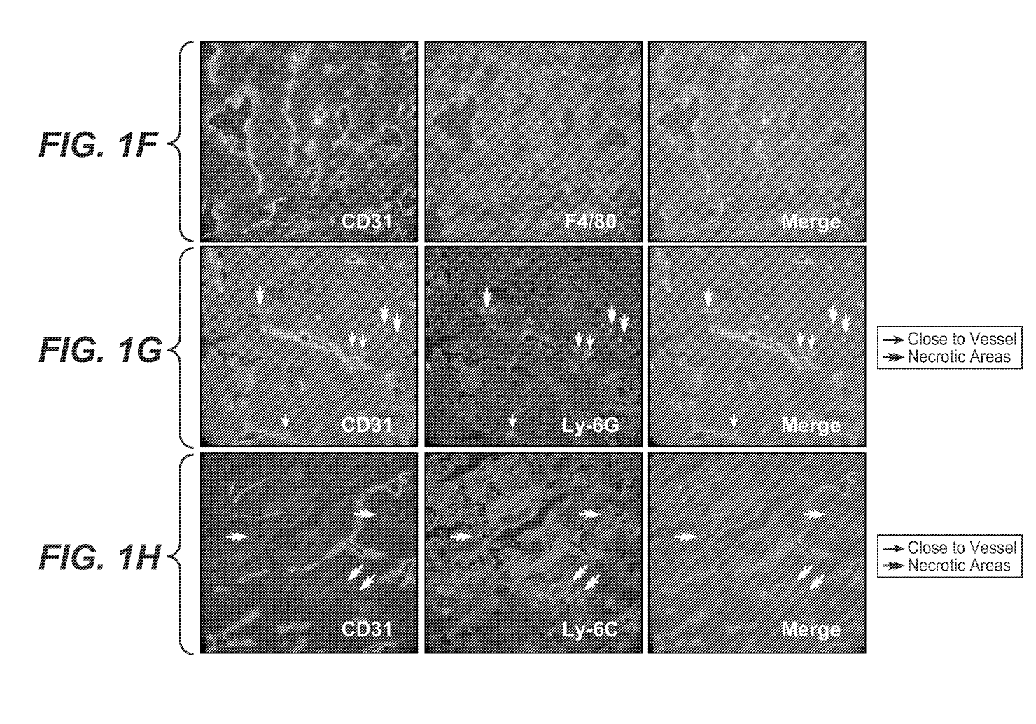

Tumor samples were first treated with an anti-CD45 antibody to detect leukocytes. As is shown in FIG. 1A, a prominent leukocyte infiltrate was identified within the tumor and the stroma. To further elucidate the composition of the infiltrate, samples were treated with anti-F4/80 antibodies or anti-CD3 antibodies as markers of macrophages and T cells, respectively. The staining pattern observed upon anti-F4/80 antibody treatment was similar to that observed with the anti-CD45 antibody treatment, indicating that a major proportion of the CD45⁺ leukocytes were macrophages (compare FIG. 1B with FIG. 1A). While CD3⁺ cells were observed within the tumor infiltrate, they were less prevalent than F4/80^{high} macrophages (compare FIG. 1C with FIG. 1B).

The results were confirmed and extended by flow cytometry. Briefly, tumors were cut into pieces and digested with collagenase II, IV, and DNase (Gibco and Sigma) for 15 minutes at 37° C. Prepared tumor cell samples were treated with fluorescently labeled antibodies specific for different myeloid subsets, including anti-CD11b, anti-GR-1, anti-Nk1.1, anti-DX5, anti-MHCII, anti-CD11c, anti-F4/80, and anti-PD-L1 (Pharmingen, Serotec, and eBioscience). All cells were blocked with the appropriate sera or purified IgG prior to staining, and cells were also stained with propidium iodide to exclude dead cells, using standard techniques. Analyses were performed using a FACSCalibur or LSR II (both Becton Dickinson).

The predominant cell type in the lymphoid tumor infiltrate was CD11b⁺ cells (see FIG. 1D). An NK1.1⁻ DX5⁻ CD11b⁺ myeloid infiltrate of 8.4±1.8% was observed. The majority of those myeloid cells were Gr-1⁻ F4/80^{low} macrophages (77.9±11.3%). Of the remaining CD11b⁺ cells, 11.5±4.3% were Gr-1⁻ F4/80^{low} resident tissue monocytes (Mo^{RT}), 1.1±0.5% Gr-1⁺ inflammatory monocytes (Mo^{IF}) and 9.5±4.3% were Gr-1⁺ neutrophils (see FIG. 1E).

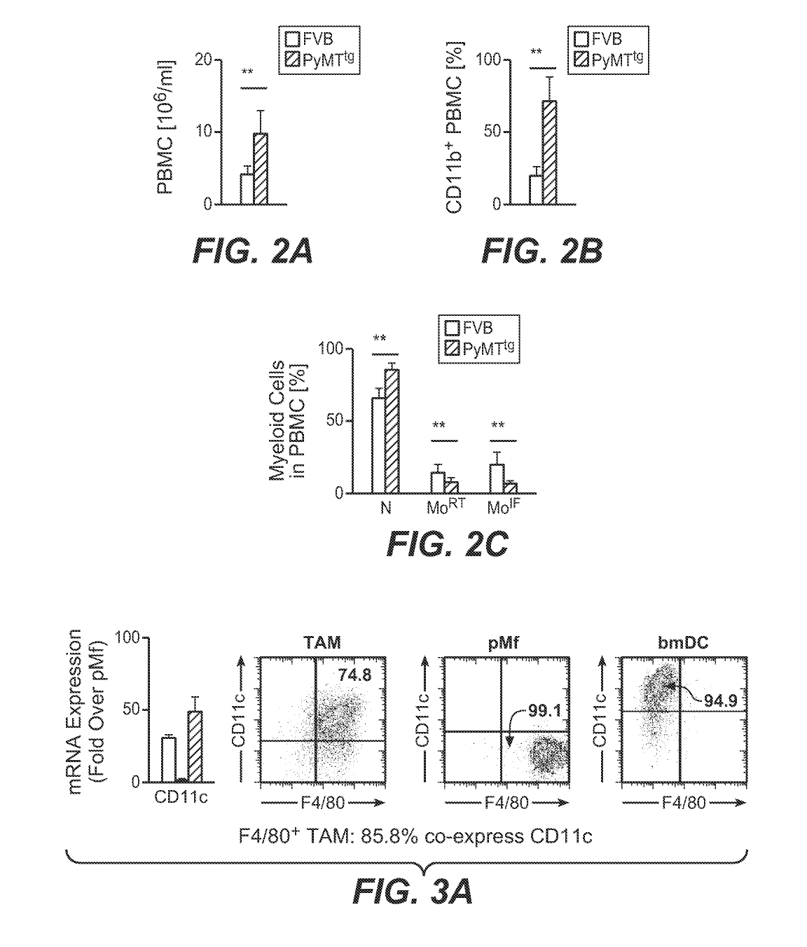

It has been reported that tumor-bearing hosts commonly exhibit leukocytosis (Serafini et al., 2004). Accordingly, the leukocyte composition in the periphery of MMTV-PyMT mice was also assessed by FACS analysis as described above. A 2.3-fold increase in the total number of peripheral blood mononuclear cells (“PBMC”) (9.7±3.2×10⁶) in mice bearing MMTV-PyMT-induced tumors was observed as compared to tumor free control FVB mice (4.2±1.1×10⁶) (FIG. 2A). This observed increase in total white blood cells in tumor-bearing mice was accompanied by an increase in the frequency of CD11b+myeloid cells (70.1±15.1%) as compared to their incidence in tumor free control mice (19.1±5.5%) (FIG. 2B). Together, the increase in total PBMC combined with the increased frequency of CD11b⁺ cells resulted in an 8.5-fold increase of peripheral myeloid cells in tumor-bearing mice. This increase was mainly due to a 12.5-fold expansion of neutrophils (6.0±1.5×10⁶/mL vs. 0.5±0.2×10⁶/mL in control mice), whereas Mo^{IF} and Mo^{RT} increased only 5-fold (0.5±0.2×10⁶/mL vs. 0.1±0.04×10⁶/mL) and 2.5-fold (0.5±0.2×10⁶/mL vs. 0.2±0.1×10⁶/mL), respectively. The ratio of neutrophils to monocytes in the blood was also slightly increased compared to that observed in control animals (FIG. 2C).

Within growing tumors the degree of vascularization is heterogeneous and regions of low oxygen tension are common and often associated with necrosis. To further understand the role of myeloid cells in tumors, immunofluorescent staining was used to localize Mo^{IF}, neutrophils and tumor associated macrophages (“TAM”) with respect to the vascular system of PyMT^{tg} tumors. Tumors from MMTV.PyMT^{tg} mice at 10-14 weeks of age were embedded in OCT solution and snap frozen. OCT frozen tumor tissues were stained with antibodies specific for F4/80 (Serotec), Ly-6C (Pharmingen), or Ly-6G (Pharmingen) (to identify TAM, Mo^{IF} or neutrophils, respectively), as well as the endothelial marker CD31 (Pharmingen) (to visualize any blood vessels in the tissue) using standard procedures (see, e.g., Example 1). The results are shown in FIGS. 1F-H. The images illustrate that F4/80⁺ TAM localize in close proximity to endothelial cells and necrotic areas of the tumor (see FIG. 1F). Similarly, neutrophils were detected close to endothelial cells and also in necrotic areas of the tumor (see FIG. 1G). In contrast, Mo^{IF} were localized almost entirely within or near necrotic areas of the tumor (see FIG. 1H).

It had previously been suggested that monocytes migrate to hypoxic regions of tumors and differentiate into macrophages (Yamashire et al., 1994; Murdoch et al., 2004). It is known that in response to hypoxia, TAM upregulate the expression of the hypoxia-induced factors HIF-1a and HIF-2a, which in turn alter TAM angiogenic, metabolic, and phagocytic activities (Mantovani et al., 2006; Lewis and Murdoch, 2005). Notably, Mo^{IF} and Mo^{IF}-derived macrophages cultured in vitro under hypoxic conditions secreted much higher levels of VEGF-A than Mo^{RT} and Mo^{RT}-derived macrophages (data not shown).

### Example 2

### Characterization of TAM

A. TAM Express CD11c and Langerin and Display Features of Professional Antigen-Presenting Cells

Both macrophages and dendritic cells (“DC”) have the ability to capture antigens and to present them to T cells. To better understand the role of TAM in the regulation of T cell responses, the expression of genes often associated with antigen presentation within tumors was assessed. Immunohistochemical analyses for markers typically expressed on myeloid or DC cells were performed on TAM according to the methods described in Example 1. Rat anti-F4/80 antibody was obtained from Serotec, rat anti-CD11b antibody was obtained from eBioscience, and rat anti-CD11c antibody was obtained from Pharmingen. Immunohistochemistry for anti-human langerin (CD207) was performed generally as described in Example 1, but the tissue sections were dewaxed and subjected to antigen retrieval in Target Retrieval buffer (pH 6.0, Dako Cytomation) using Lab Vision's PT Module at 99° C. for 20 minutes with subsequent cooldown for 20 minutes. Goat anti-langerin was obtained from R&D Systems, and biotinylated rabbit anti-goat IgG was obtained from VectorLabs.

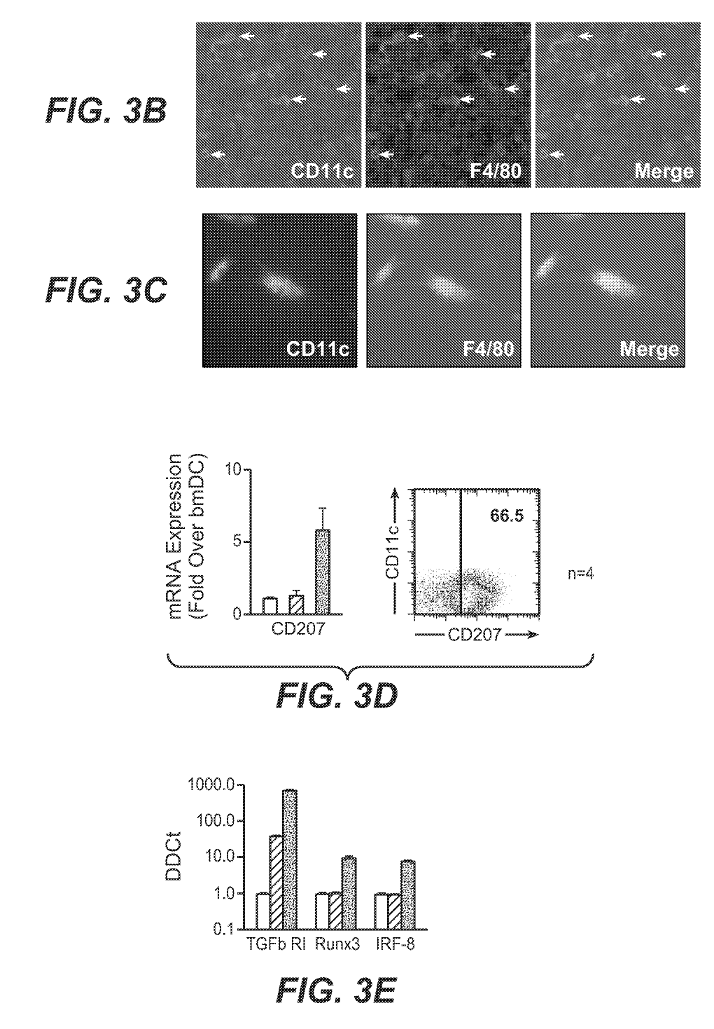

With a few exceptions, the majority of tissue-resident macrophages (e.g., peritoneal macrophages) are CD11b⁺ F4/80⁺ CD11c⁻ cells, while myeloid DC (e.g., bone-marrow-derived DC) are CD11b⁺ CD11c⁺ cells and lack expression of F4/80. Surprisingly, it was observed that the CD11b⁺ TAM from PyMT-derived tumors expressed not only F4/80 at the cell surface, but also high levels of CD11c (FIG. 3A). Similar results were observed in TAM isolated from MMTV-HER2^{tg} mice (data not shown). Histology of OCT frozen tumors from PyMT^{tg} mice showed that TAM co-express F4/80 and CD11c (FIG. 3B), further confirmed by immune fluorescence studies of isolated TAM cultured for 60 hours in vitro (FIG. 3C). Also surprisingly, TAM from PyMT^{tg} mice also expressed the C-type lectin langerin, a protein thus far known to be mainly expressed by Langerhans DC (LhDC) (Kissenpfennig and Milissen, Trends Immunol 27: 132-9, 2006; Kaplan et al., Immunity 23: 611-20, 2005) (FIG. 3D).

Since the development of murine and human Langerhans DC (LhDC) is dependent on TGFβ1 signaling (Borkowski et al., J Exp Med 184: 2417-22, 1996; Jaksits et al., J Immunol 163: 4869-77, 1999), the expression of this cytokine was investigated in TAM. Indeed, TAM expressed higher levels of mRNA encoding TGFβ R1 (ΔΔct=707.3±47.3) compared to the amount observed in bmDC (ΔΔct=1.0±0.06) and peritoneal macrophages (ΔΔct=39.9±1.6). It was further observed that TAM expressed comparatively higher levels of mRNA encoding Runx3 (TAM: ΔΔct=9.8±1.1; bmDC: ΔΔct=1.0±0.06; peritoneal macrophages: ΔΔct=1.02±0.05) and IRF-8 (TAM: ΔΔct=7.9±0.6; bmDC: ΔΔct=1.0±0.06; peritoneal macrophages: ΔΔct=0.98±0.05) (FIG. 3E), two transcription factors involved in LhDC development and in the TGFβ signaling cascade (Woolf et al., Dev Biol 303: 703-14, 2007; Schiavoni et al., Blood 103: 2221-8, 2004). Runx3 has been shown to regulate expression of CD11c, and both Runx3.KO and IRF-8.KO mice are deficient in the generation of LhDCs (Borkowski et al., J Exp Med 184: 2417-22, 1996). Taken together, the data suggests that TGFβ is an important factor for the observed TAM biology.

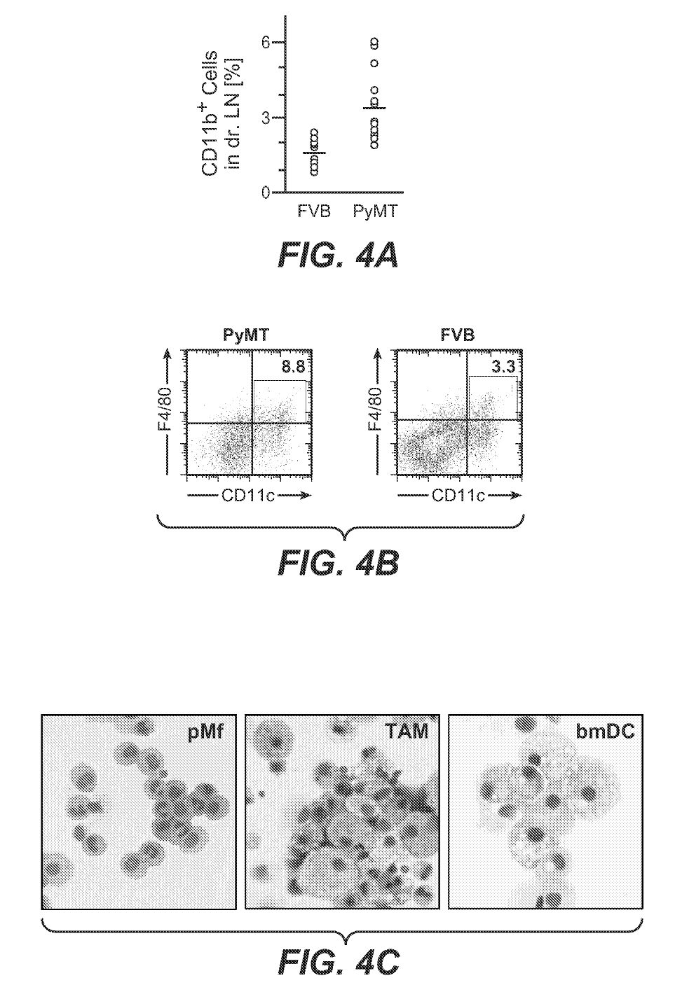

Professional antigen presenting cells are known to migrate to draining lymph nodes to initiate immune reactions. Accordingly, the immune cell composition of tumor draining axillary and brachial lymph nodes of PyMT^{tg} mice was assessed in comparison with control lymph nodes from tumor-free FVB mice using FACS analysis as described in Example 1. Elevated numbers of CD11b⁺ cells (FIG. 4A), as well as elevated number of CD11b⁺ cells expressing F4/80 and CD11c (FIG. 4B) were identified in the lymph nodes from the tumor-containing mice. One nonlimiting interpretation of this data is that TAM might migrate to the draining lymph nodes to present tumor antigens to other immune cells.

A comparative full-genome microarray analysis was performed to further investigate the differences between TAM and peritoneal macrophages and bone marrow-derived dendritic cells (“bmDC”) from FVB control mice. Briefly, one microgram of total RNA was converted into double-stranded cDNA using a Low RNA Input Fluorescent Linear Amplification Kit (Agilent). cRNA was synthesized from cDNA using T7 RNA polymerase, simultaneously incorporating cyanine 3- or cyanine 5-labeled CTP. The labeled cRNA was purified on an affinity resin column (RNeasy Mini Kit, Qiagen), and quantified by measuring absorbance at 260 nm. Incorporation of dye was determined by measuring the absorbance of cyanine 3- and cyanine 5-labeled CTP using a NanoDrop ND-1000 Spectrophotometer (NanoDrop Technologies). 750 ng of cyanine 3-labeled cRNA and 750 ng of cyanine 5-labeled cRNA was fragmented by incubation at 60° C. for 30 minutes in fragmentation buffer (In situ Hybridization Kit-Plus; Agilent). Fragmentation was terminated by the addition of hybridization buffer containing LiCl and lithium lauryl sulfate. Samples were hybridized to microarrays at 60° C. for 17 hours. Arrays were washed with SSC buffer and dried with acetonitrile. Arrays were scanned using a Microarray Scanner (Agilent).

Immature bmDC were generated from red blood cell-depleted bone marrow cells, cultured at 5×10⁵ cells/mL in RPMI 1640 medium (Sigma-Aldrich) supplemented with 150 ng/mL murine IL-4 and 20 ng/mL murine GM-CSF (R&D Biosystems) at 37° C. with 5% CO₂ for six days. Every second day half of the medium was removed and replaced with fresh RPMI 1640 supplemented with GM-CSF and IL-4. At day six CD11b⁺ CD11c⁺ cells were isolated by FACS sorting. F4/80^{high} peritoneal macrophages were FACS sorted from single cell solutions obtained from peritoneal lavages with PBS/EDTA. Tumors from 10-14 week old MMTV.PyMT^{tg} mice were digested with collagenase II, IV, and DNase (Gibco and Sigma) for 15 minutes at 37° C. Tumor-associated F4/80^{high} macrophages (TAM) were enriched by magnetic cell sorting using anti-F4/80 PE and anti-PE MicroBeads (Miltenyi Biotech). The purity of the sorted cells was verified by flow cytometry and ranged greater than 95% for cells purified by magnetic cell sorting and greater than 98% for cells purified by flow cytometry. All cells were blocked with 10-20% of the appropriate sera or purified IgG prior to staining. FACS sorting was conducted with PI exclusion on either a Vantage or Aria sorter (Becton Dickinson). Hierarchical clustering and principal component analysis (PCA) were performed by using Partek® Genomic Suite TM software, version 6.3 (Partek Inc., St. Louis, Mo.) on Agilent Whole Mouse Genome (WMG) or MIA (comparison of macrophage subsets) Oligo Microarray log 2 ratio data (Agilent Technologies Inc., Santa Clara, Calif.). Euclidean distance was used to measure dissimilarities between rows or columns, average linkage method to calculate distances between clusters and “2-Pass” clustering method in the hierarchical clustering. In PCA, the dispersion matrix is covariance, and eigenvectors are normalized. The Partek Batch Remover was used to remove the effect of the mouse strain difference on data visualization in PCA. The expression values of Agilent log 2 ratio were converted to z-scores in the intensity plots.

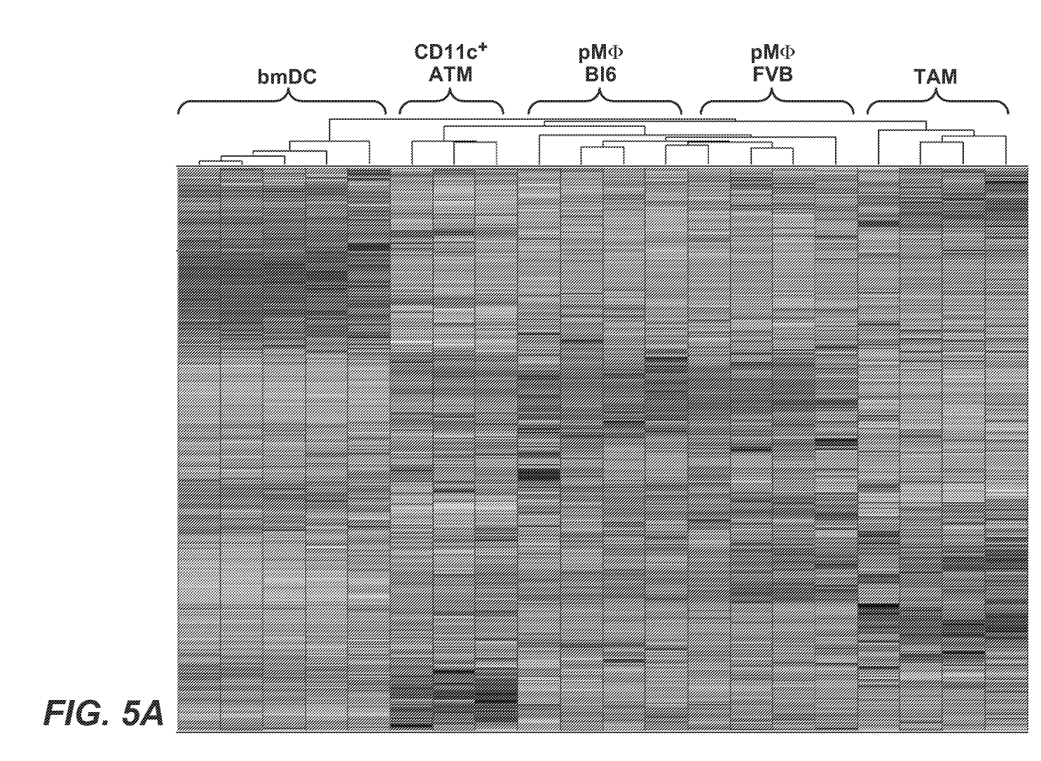

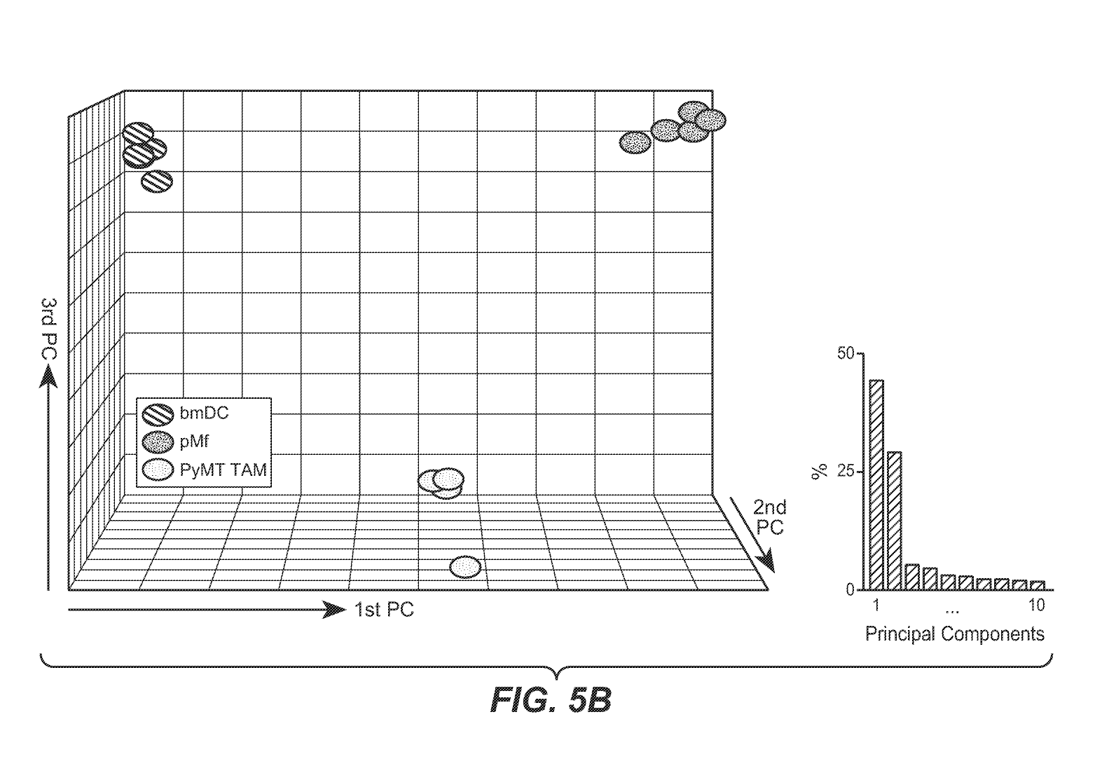

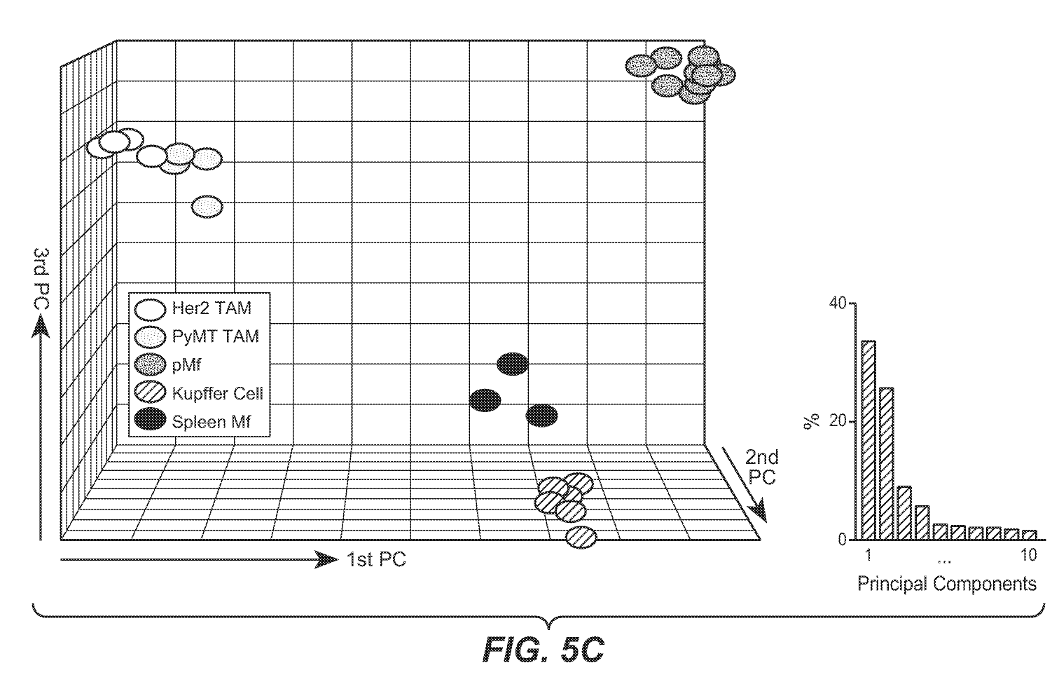

A heatmap image of expressed genes in those three cell types shows TAM to be distinct from peritoneal macrophages and bmDC (FIG. 5A). The data were also examined statistically by three-dimensional principal component analysis (“PCA”) to estimate the relationships between the three different gene expression profiles. The clustering of the populations showed TAM to be distinct from both control populations, although TAM seemed to be more related to peritoneal macrophages than to bmDC (FIG. 5B). FIG. 5C shows that TAM are differentiable from other macrophages such as peritoneal macrophages, splenic macrophages, and Kupffer cells.

The morphology of TAM was also compared to that of peritoneal macrophages and bmDC (FIG. 4C). TAM and bmDC were large cells having small nuclei and large cytoplasms interspersed with many vacuoles. In contrast, peritoneal macrophages were much smaller in size and had large nuclei and a homogenous cytoplasm lacking vacuoles. Thus TAM looked markedly different than peritoneal macrophages, but similar to bmDC.

B: TAM Display Features of Tolerogenic Antigen-Presenting Cells

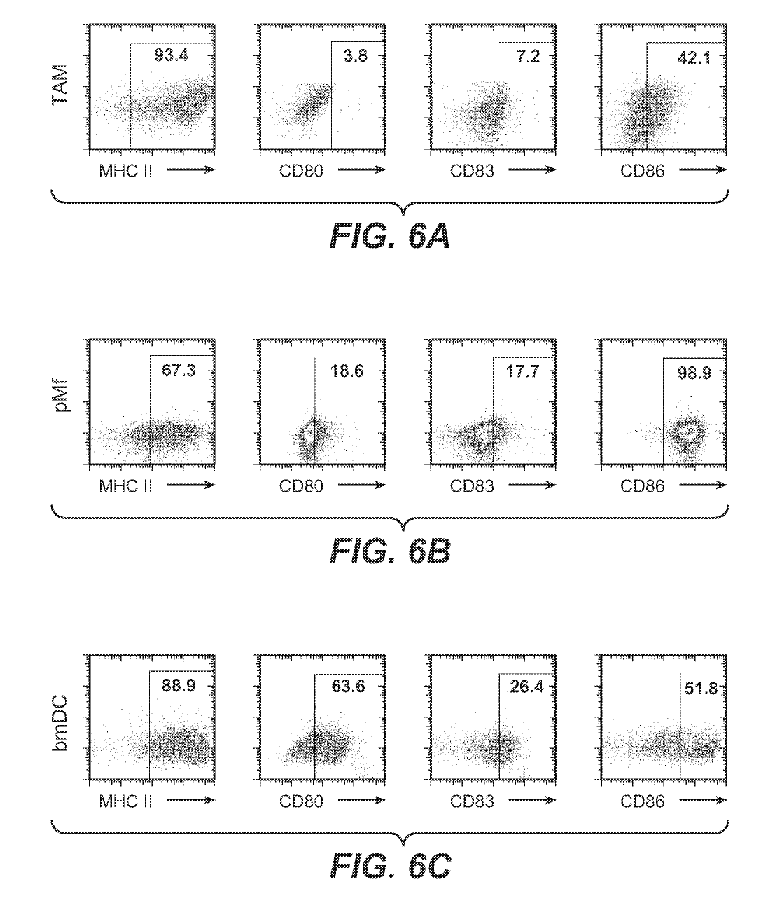

Given the morphological similarities of TAM isolated from PyMT^{tg} mice to bmDC, and dissimilarity to peritoneal macrophages, described above, further analysis of the molecular similarities and differences between these cell types was performed, particularly to assess whether TAM isolated from PyMT tumors might act as antigen-presenting cells. The expression of MHC II, the co-stimulatory molecules CD80 and CD86, and CD83 (a marker for mature DC) was measured in TAM, bmDC, and peritoneal macrophages by FACS analysis as described above. Notably, TAM expressed MHC II at high levels, similar to those observed on semi-mature bmDC, while peritoneal macrophages only expressed moderate levels of MHC II (compare leftmost panels in FIGS. 6A-C). TAM expressed little to none of CD80 or CD83, and a moderate amount of CD86. Resting peritoneal macrophages expressed low levels of CD80 and CD83, but high levels of CD86, while bmDC, a heterogeneous population of immature and semi-mature DC, expressed low to moderate levels of CD80, CD83, and CD86 (FIGS. 6A-C).

### Example 3

### TAM Chemokine and Cytokine Profile

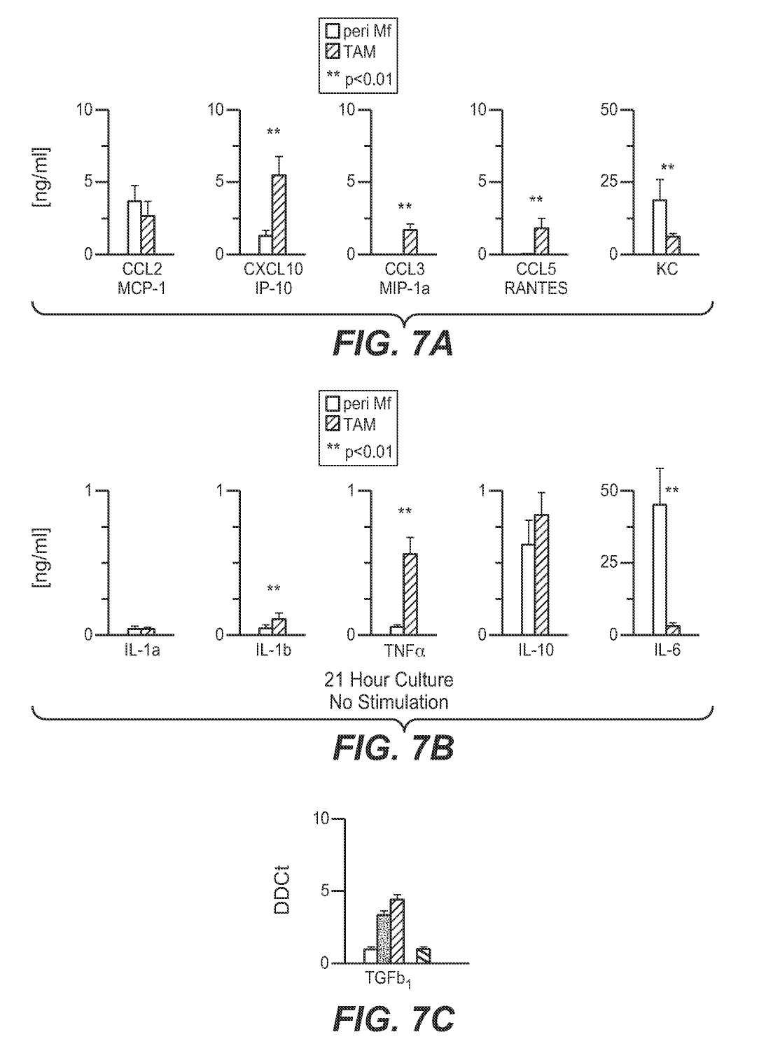

To further understand how TAM might influence tumor growth and progression as well as anti-tumor immune response, the cytokine and chemokine profiles of TAM were assessed. Microarray analyses were performed as described in Example 2 for a selected set of genes: chemokines CCL2, CCL3, CCL4, CCL5, CCL7, CCL8, CCL17, CXCL1, CXCL9, CXCL10, CXCL16, and KC and cytokines IL-1α, IL-1β, IL1 RA, TNFα, TGFβ, and LTβ. Peritoneal macrophages and TAM displayed distinct chemokine and cytokine profiles (see Table 2 and FIG. 7A). TAM produced larger amounts of mRNA encoding certain chemokines, for example CCL2, CCL3, CCL4, CCL5, CCL7, CCL8, CCL17, CXCL1, CXCL9, CXCL10, CXCL16, and KC (see Table 2 and FIG. 7A) as compared to bmDC. Such chemokine expression should attract a variety of lymphocytes, including those typically found in tumors such as monocytes, immature DC, NK cells and T cells. Enhanced levels of mRNA encoding IL-1α, IL-1β, IL-1 RA, TNFα and LTβ in TAM were detected in comparison to bmDC (data not shown).

TABLE 2
Chemokine mRNA Expression in TAM as Compared to bmDC

| Chemokine | Expression | Target Cells |
|---|---|---|
| CCL2/MCP-1 | 12.4 | Monocytes, memory T cells (CD8), |
| | | immature DC |
| CCL3/MIP-1a | 37.9 | Monocytes, NK cells, memory T cells |
| | | (TH1), immature DC |
| CCL4/MIP-1b | 183.3 | TH1 |
| CCL5/RANTES | 5.0 | TH1, NK cells, immature DC |
| CCL7/MCP-3 | 15.2 | Monocytes |
| CCL8/MCP-2 | 5.1 | Monocytes |
| CCL17/TARC | 4.0 | TH2, regulatory T cells |
| CXCL1/MIP-2* | 5.5 | Monocytes, NK cells |
| CXCL9/MIG | 5.6 | TH1 |
| CXCL10/IP-10 | 103.0 | TH1, monocytes, activated T cells |
| | | (TH1, TH2) |
| KC | 6.2 | Neutrophils |
| CXCL16* | 13.3 | T cells |

*transmembrane protein; shedded forms act as scavenger receptors

Heatmap analyses of gene expression in TAM as compared to tumor cells, peritoneal macrophages, and bmDC were also performed. Total RNA was purified using an RNeasy mini kit (Qiagen) according to the manufacturer's instructions. RNA quality was evaluated using the Total RNA Pico Assay on an Agilent 2100 Bioanalyzer, and a Low RNA Input Fluorescent Linear Amplification Kit was used to prepare fluorescent cRNA probes (Agilent). Agilent Mouse M1A microarrays were used to evaluate gene expression. The six replicate samples for each cell type were labeled with Cy5 and Universal Mouse Reference (Stratagene) was labeled with Cy3. 750 ng of labeled Cy5 and Cy3 probes were fragmented for 30 min and both probes were loaded on each chip. Overnight hybridization was performed at 60° C., and slides were subsequently washed in 6×SSC and 0.1×SSC, followed by an acetyl nitrile drying step. Microarrays were scanned with a scanner sensitivity set to 100.

The results showed that TAM expressed elevated levels of mRNA encoding a number of inflammatory (IL-1α, IL-1β, TNFα and LTβ) as well as anti-inflammatory cytokines (IL-1RA, IL-10, and TGFβ1), but low levels of mRNA encoding IL-6, TGFβ2 or TGFβ3 (FIG. 14A). These analyses also found that TAM exhibit a unique cytokine receptor expression pattern with elevated levels of IL-4Rα, IL-10Rα, IL-10Rβ, IL-13Rα, IL-17Rα, TGFβR1 and TGFβR2 (FIG. 14B). TAM also expressed elevated levels of mRNA encoding many inflammatory chemokines (CCL2, CCL12, CCL3, CCL4, CCL7, CCL12, CXCL1, CXCL2, CXCL9, CXCL10, CXCL11, CXCL14 and CXCL16) (FIG. 14C). Purified TAM secreted high levels of CCL3 (1.1±0.3 ng/ml versus an undetectable amount in peritoneal macrophages), CCL5 (1.8±0.6 ng/ml versus an undetectable amount in peritoneal macrophages), and CXCL10 (5.5±1.3 ng/ml versus 1.3±0.3 ng/ml in peritoneal macrophages), while expression of CCL2 was similar to that of peritoneal macrophages (2.7±1.0 ng/ml versus 3.9±0.9 ng/ml) (FIG. 14D). This distinct chemokine profile suggested that TAM actively recruit leukocytes to tumors. TAM also expressed elevated levels of mRNA encoding CCR6, CXCR4 and CX3CR1, chemokine receptors known to be induced by TGFβ1 (Chen, S. et al., Immunology 114: 565-74, 2005; Yang, D., et al., J Immunol 163: 1737-41, 1999; Chen, S., J Neuroimmunol 133: 46-55, 2002), as well as elevated levels of CCR2, CCR12 and CCR5 (FIG. 14E). Real-time RT-PCR confirmed the presence of elevated CCR6 mRNA levels in TAM (TAM: ΔΔct=467.8±332.0; bmDC: ΔΔct=1.0±0.6; peritoneal macrophages: ΔΔct=6.4±6.1) (FIG. 14F).

To test whether this distinct chemokine and cytokine mRNA profile observed in TAM is also present at the expressed protein level, TAM were purified from PyMT^{tg} mice as described above and the production of certain cytokine and chemokine proteins was assessed after 21 hours of culture in comparison to protein expression in peritoneal macrophages. FACS analysis was performed as described in Example 1. TAM and peritoneal macrophages were cultured in fibronectin-coated round-bottom 96 well-plates for 21 hours at a concentration of 2×10⁶/mL in RPMI1640 medium at 37° C. and 5% CO₂. Cytokines secreted in the supernatant were detected by Luminex analysis. Real-time RT-PCR analyses were also performed. RNA of sorted immune cells was isolated with an RNeasy kit (Qiagen) and digested with DNase I (Sigma). Total cellular RNA was reverse transcribed and analyzed by real-time TaqMan PCR in triplicates with a 7700 Sequence Detection System (Applied Biosystems) according to the manufacturer's instructions. Arbitrary expression units of the expressed genes were given as fold-expression of that of the housekeeping gene GAPDH. Primers to individual genes were designed over exon/intron borders according to standard protocols and were obtained from Applied Biosystems.

The results are shown in FIG. 7B. While TAM and peritoneal macrophages both secreted moderate levels of IL-10 (0.81±0.13 ng/mL in TAM versus 0.69±0.19 ng/mL in peritoneal macrophages), TAM produced relatively high levels of TNFα (0.57±0.12 ng/mL in TAM versus 0.08±0.01 in peritoneal macrophages) and very low levels of IL-6 (3.5±0.5 ng/mL in TAM versus 48.5±12.7 ng/mL in peritoneal macrophages). Additionally, TAM secreted low levels of IL-1α (0.05±0.01 ng/mL in TAM vs. 0.05±0.02 ng/mL in peritoneal macrophages) with slightly, but significantly elevated levels of IL-1β (0.12±0.04 ng/mL versus 0.05±0.02 ng/mL).

Chemokine analysis for the most part confirmed the distinct TAM profile observed by the above microarray analyses. TAM expressed high levels of mRNA for CCL3 (1.1±0.3 ng/mL in TAM, undetectable in peritoneal macrophages); CCL5 (1.8±0.6 ng/mL in TAM, undetectable in peritoneal macrophages; and CXCL10 (5.5±1.3 ng/mL in TAM versus 1.3±0.3 ng/mL in peritoneal macrophages) (FIG. 7B). Expression of CCL2 mRNA in TAM was similar to that of peritoneal macrophages (2.7±1.0 ng/mL in TAM versus 3.7±1.0 ng/mL in peritoneal macrophages) while expression of KC mRNA (6.4±0.7 ng/mL in TAM versus 18.6±6.9 ng/mL in peritoneal macrophages) was diminished (FIG. 7B). Real-time PCR analyses of TGFβ1 expression in PyMT^{tg}-derived TAM, peritoneal macrophages, bmDC, and tumor cells showed that TAM have the highest expression of that cytokine (FIG. 7C). The combination of moderate expression of TGFβ, IL-10, and TNFα with very low levels of IL-6 suggests that TAM may have immune suppressive properties. Furthermore, the observed TAM chemokine profile suggests that TAM may be able to modulate leukocyte infiltrates observed in tumors by secreting a wide variety of chemokines.

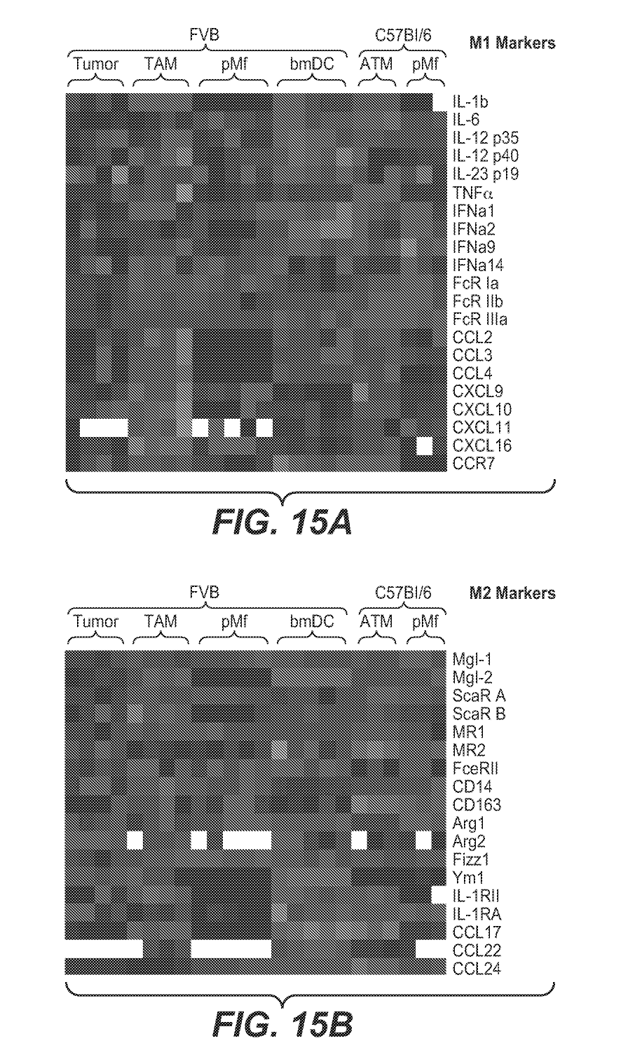

The literature-recognized M1/M2 paradigm (see Mantovani et al., Trends Immunol 25 (12): 677-86, 2004; Gordon, Nat Rev Immunol 3 (1): 23-35, 2003) suggests that macrophages under either classical inflammatory (IFNγ/LPS) or alternative activated (IL-4/IL-13) conditions differentiate into specialized subsets (M1, respectively M2) with unique functional properties. It has been proposed that classical M1 macrophages support inflammatory reactions, whereas M2 macrophages stimulate the development of a suppressive IL-10 and TGFβ-rich microenvironment. The literature has classified TAM as “alternatively activated” M2 macrophages (Mantovani et al., Trends Immunol 23: 549-55,2002; Sica et al., Eur J Cancer 42: 717-27, 2006). A heatmap analysis of mRNA expression profiles of molecules associated with either an M1 or M2 phenotype found that TAM express elevated mRNA levels of certain M2-associated molecules (ScaR B, MR1, CD14, CD163, Fizz 1, IL-1RII and IL-1RA) in comparison with neutral peritoneal macrophages, but lacked expression of other M2-associated molecules (Mgl1, Mgl2, ScaR A, MR2, FceRII, Arg1, Ym1, CCL17, CCL22 and CCL24) and also express elevated mRNA levels of M1-associated molecules (IL-1β, FcRIa, FcRIIb, FcRIIIa, CCL2, CCL3, CXCL9, CXCL10, CXCL11 and CXCL16 (FIGS. 15A-B). These observed cytokine and chemokine profiles demonstrate that TAM, although secreting suppressive cytokines, are distinct from M2 macrophages. TAM show many inflammatory M1 characteristics, such as the production of TNFα and IL-1β and the expression of FcRI, FCRIIb and FcRIIIa. TAM secreted many inflammatory “M1” CC and CXC chemokines chemotactic to, for example, NK cells, but note of the classic M2 chemokines CCL17, CCL22 and CCL24 which attract TH2 or T regulatory cells.

### Example 4

### TAM Effect on T Cells In Vitro

To investigate whether the above-described properties of TAM reflect TAM interaction with T cells, the capacity of TAM to induce naïve T cell proliferation and cytokine secretion was assessed in comparison with the T-cell induction activities of peritoneal macrophages and bmDC. Although the PyMT^{tg} tumor model mimics many aspects of human metastatic breast cancer development, it also necessitates the FVB background, making it difficult to perform antigen-specific T-cell studies. Instead, in vitro co-cultures with the selected immune cells and CFSE-labeled CD4⁺ T cells were employed. Naïve CD4⁺ T cells were prepared from spleen and peripheral lymph nodes of FVB mice. Single cell suspensions were MACS depleted of CD25⁺, CD69⁺, and CD103⁺ cells (eBioscience and Miltenyi Biotech). CD4⁺ T cells in the negative fraction were enriched with CD4-MicroBeads (Miltenyi Biotech) and CD62L⁺CD45Rb^{high}CD25⁻CD69⁻CD103⁻ naïve CD4⁺ T cells were isolated by FACS sorting (all antibodies from eBioscience or Pharmingen). T-cell proliferation induced by TAM, peritoneal macrophages, or bmDC was investigated by culturing 2×10⁴ TAM, peritoneal macrophages, or bmDC with 1×10⁵ naïve T cells with 0.5 μg/mL of anti-CD3 antibodies. The cells were cultured in fibronectin coated round-bottom 96 wells at 37° C. with 5% CO₂. After five days of culture the cellular supernatants were frozen at −80° C. for cytokine analysis by ELISA assay using standard procedures. GM-CSF, G-CSF, MIP-1α, MCP-1, RANTES, IP-10, KC, IL-1α, IL-1β, IL-2, IL-4, IL-5, IL-6, IL-10, IL-13, IL-17, IFNγ and TNFα were detected in culture supernatants with Lincoplex kits (Linco) following the manufacturer's instructions. T cell proliferation was examined by FACS.

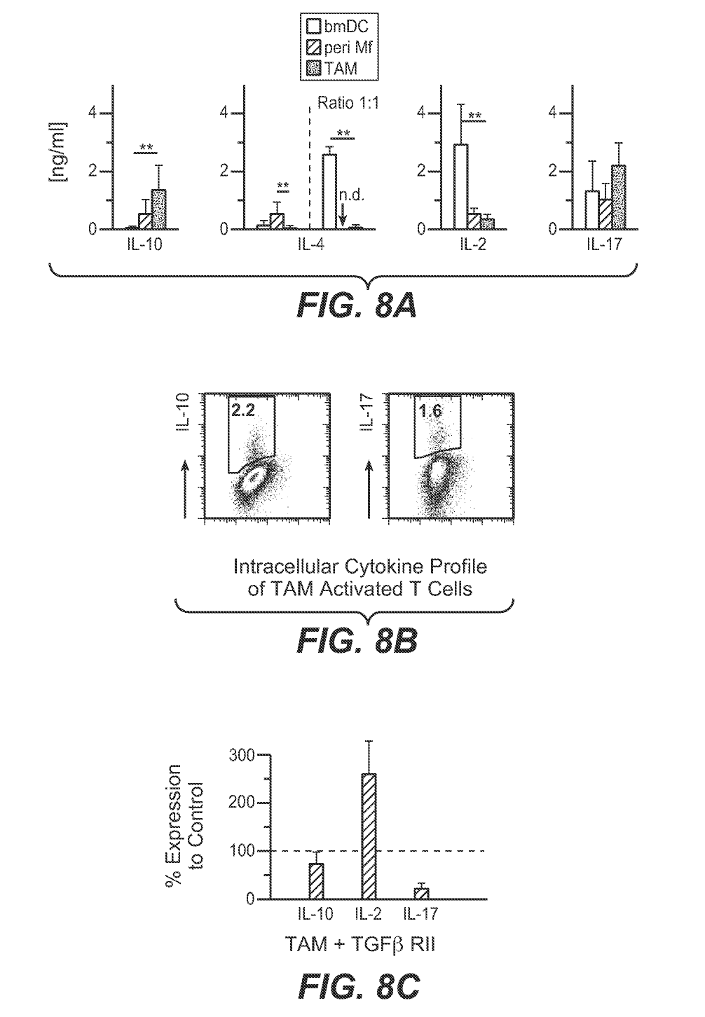

Measurements of dilution of CSFE showed that TAM induced T cell proliferation at levels comparable to those induced by the professional antigen presenting cells peritoneal macrophages and bmDC (data not shown). In contrast to bmDC, TAM-primed T cells secreted high levels of IL-10 (2.5±0.4 ng/mL in TAM-primed cells versus undetectable in bmDC-primed cells) and IFNγ (5.6±1.2 ng/mL in TAM-primed cells versus 0.2±0.1 ng/mL in bmDC-primed cells), combined with very low levels of IL-2 (0.3±0.1 ng/mL in TAM-primed versus 5.4±0.6 ng/mL in bmDC-primed cells) and no IL-4 (undetectable in TAM-primed versus 0.2±0.1 ng/mL in bmDC-primed cells) (FIG. 8A). The inability of TAM to induce IL-4 in naïve CD4⁺ T cells was more dramatic at a ratio of 1:1, showing a near-complete lack of induction of IL-4 by TAM priming (0.2±0.02 ng/mL) (FIG. 8A) as compared to very high IL-4 induction by bmDC (2.7±0.2 ng/mL). T cells stimulated with TAM or bmDC expressed comparable levels of IL-5, IL-13, and TNFα (data not shown). This pattern of cytokine secretion from CD4⁺ T cells activated by TAM suggested that TAM induces IL-10⁺ Trl T cells. TAM also induced high levels of IL-17 (1.6±0.6 ng/mL in TAM-primed versus 0.7±0.1 ng/mL in bmDC-primed T cells) (FIG. 8A, right panel).

To confirm that those T-cell cytokines were secreted by the T cells, TAM and other immune cell-activated CD4⁺ T cell cultures (as described above) were fixed and stained intracellularly for each major cytokine. Briefly, T cells cultured for 5 days with TAM, peritoneal macrophages, or bmDC were restimulated with 50 ng/mL PMA and 750 ng/mL ionomycin for six hours with the addition of 5 μg/mL Brefeldin A for the last four hours, then treated with blocking reagents and surface-stained for CD4. Cells were then fixed and stained for intracellular expression of IL-4, IL-10 and IL-17 using a FastImmune™ CD4 Intracellular Cytokine Detection Kit (BD) according to the manufacturer's instructions. FACS analysis was performed as described in Example 1. As shown in FIG. 8B, TAM-primed CD4⁺ T cells secreted high levels of IL-10 and IL-17 and diminished levels of IL-2, and this secretion was dependent on TGFβ secretion by TAM. Neutralization of TGFβ by the addition of recombinant TGFβRII resulted in a decrease in IL-10 (27.4±22.1%) and IL-17 (79.4±9.9%) expression and a 259.9±68.0% increase of IL-2 secretion (FIG. 8C). This data, in conjunction with the findings above, confirmed that TAM was likely inducing IL-10⁺ Trl and IL-17⁺ CD4⁺ T cells in those cultures.

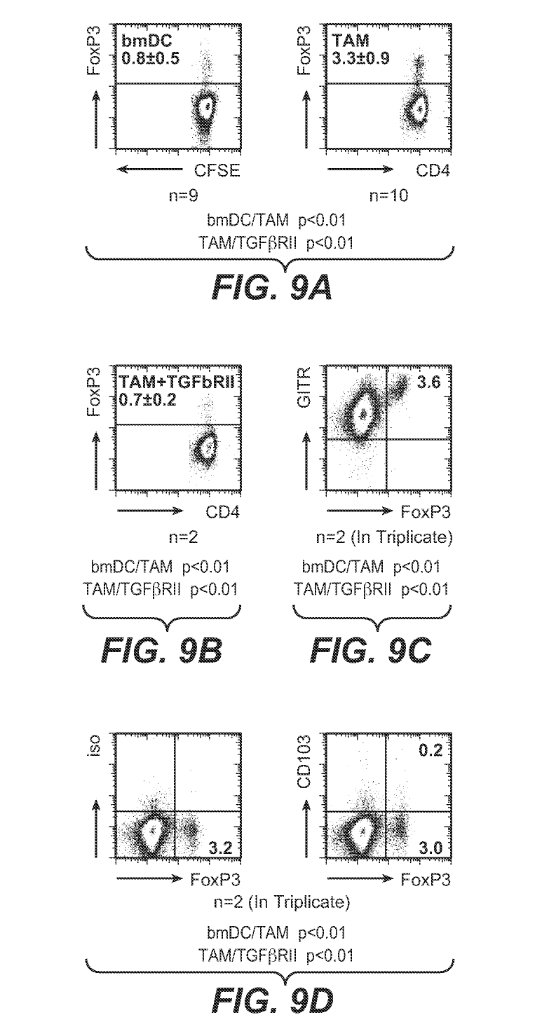

Having shown that TAM likely induce at least one certain regulatory T cell subset, experiments were performed to determine if TAM were also able to induce FoxP3⁺ regulatory T cells. To assess FoxP3 induction, 2×10⁴ TAM or bmDC were cultured with 1×10⁵ naïve CDSE⁺ T cells (CD25⁻ CD69⁻ CD103⁻ CD45Rb^{high} CD62L^{high} and almost negative for FoxP3 (0.3% FoxP3⁺)) and 0.002 μg/mL anti-CD3 antibody in fibronectin-coated round-bottom 96 well plates at 37° C. with 5% CO₂. After five days intracellular FoxP3 expression by CD4⁺ T cells was analyzed using a conjugated antibody specific for mouse and rat FoxP3 (eBioscience), following the manufacturer's instructions. The results are shown in FIG. 9. In contrast to bmDC activated CD4⁺ T cells, TAM activation favored induction of regulatory T cells, as evidenced by the presence of 3.3±0.9% FoxP3⁺ T cells in TAM-treated cultures, whereas bmDC-treated cultures only showed 0.8±0.5% of FoxP3⁺ T cells (see FIG. 9A) FoxP3 induction in T cells activated with TAM was also dependent on TGFβ production by TAM, since the neutralization of TGFβ with recombinant TGFβRII diminished the expression of FoxP3 in TAM treated T cells by almost 80% to 0.7±0.2% of FoxP3⁺ T cells (FIG. 9B).

To confirm that TAM-induced FoxP3⁺ T cells have regulatory capacity, the cells were stained for proteins known to be expressed on naturally occurring FoxP3⁺ regulatory T cells. It was known that GITR is expressed at higher levels on FoxP3⁺ regulatory T cells as compared to other CD4⁺ T cells (McHugh et al., Immunity 16: 311-23, 2002). TAM-induced T cells also had high levels of GITR expression (FIG. 9C), suggesting that those cells are FoxP3⁺ T cells with regulatory capacity. Also, some TAM-induced FoxP3⁺ T cells (approximately 6.3%, see FIG. 9D) express low levels of CD103, a marker known to be expressed on peripherally-induced regulatory T cells in vivo, further suggesting that TAM-induced FoxP3⁺ T cells are regulatory T cells with regulatory properties.

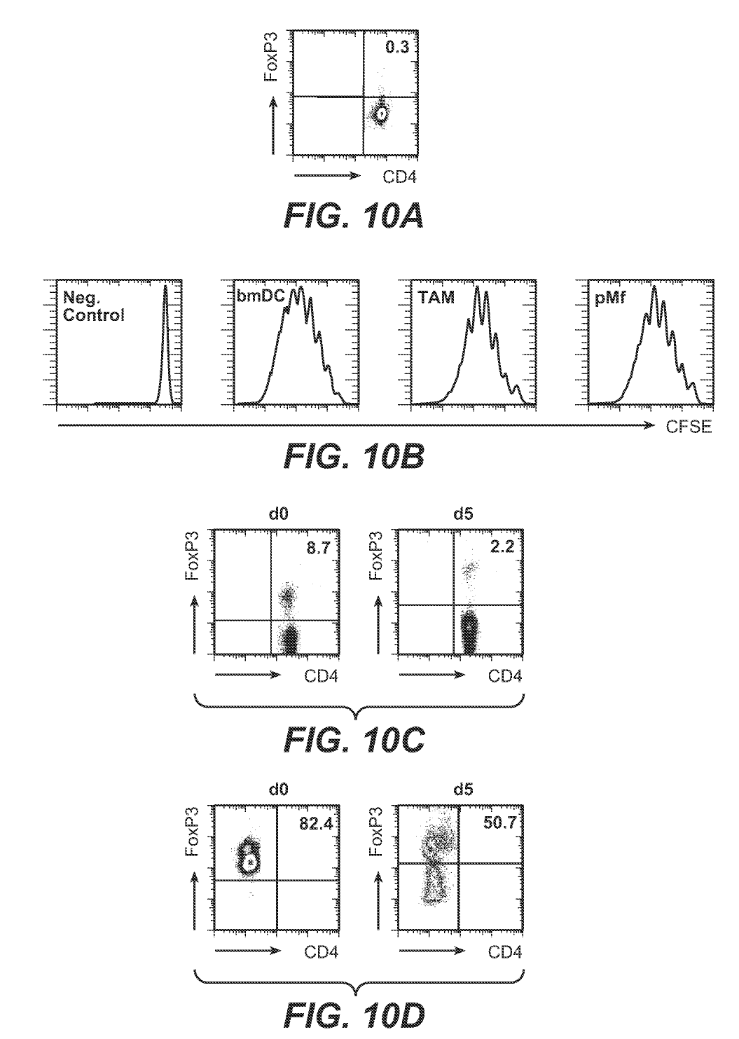

To clarify that TAM induced FoxP3⁺ T cells as opposed to merely stimulating the expansion of the few FoxP3⁺ T cells remaining in the pool of naïve T cells after isolation (0.3%, see FIG. 10A), splenocytes containing 8.7±0.2% FoxP3⁺ cells in the CD4⁺ T-cell pool were stimulated under the same conditions. As shown in FIG. 10C, the pool of FoxP3⁺ T cells was reduced to 2.2±1.5%, suggesting that TAM are able to induce FoxP3⁺ regulatory CD4⁺ T cells directly. The experiment was repeated using T regulatory cells instead of splenocytes, and the results were the same (compare FIGS. 10D and 10C). The stimulatory capacity of different antigen-presenting cells on naïve CD4⁺ T cells was also assessed. As shown in FIG. 10B, each of bmDC, TAM and peritoneal macrophages were able to stimulate CFSE labeled naïve CD4⁺ T cells to similar extents. Thus, the induction of FoxP3⁺ T cells by TAM is not due to a generalized increase in T cell induction with TAM relative to other antigen presenting cells.

### Example 5

### TAM Effect on T Cells In Vivo

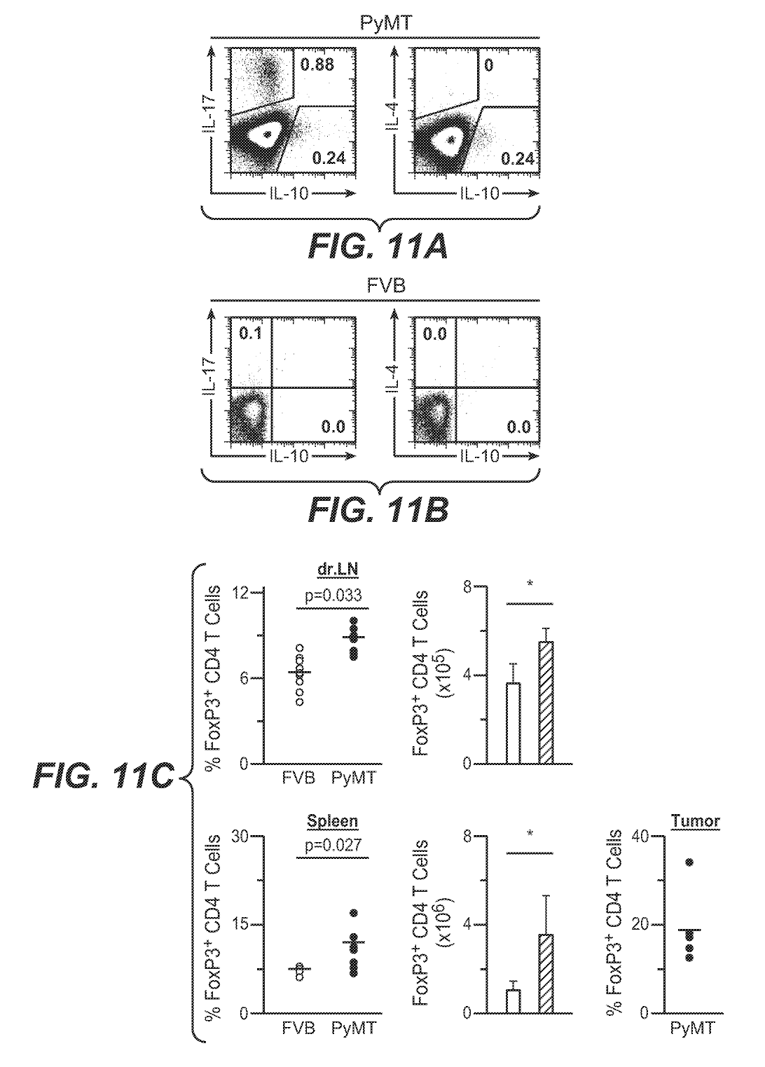

The combined data from the preceding examples suggested that TAM induce both IL-10⁺ and FoxP3⁺ regulatory T cells as well as IL-17⁺ TH_{IL-17} CD4⁺ T cells in vitro. To assess the in vivo relevance of inducing such cell populations, the presence and localization of those cell subsets in vivo was determined. Single cell suspensions of axillary and brachial lymph nodes from PyMT^{tg} mice were prepared using standard techniques. The cell suspensions were restimulated for six hours and then stained with antibodies specific for CD4, IL-4, IL-10, and IL-17 as described previously. In good agreement with the in vitro data, IL-4- and IL-10⁺ CD4⁺ Trl cells were detected in vivo, as well as IL-17⁺ CD4⁺ T cells (FIG. 11A). No significant expression of these cytokines was detected in CD4⁺ T cells from axillary and brachial lymph nodes derived from age-matched control FVB mice (FIG. 11B). Interestingly, all cytokine-producing CD4⁺ T cells expressed only one of the investigated cytokines (i.e., either IL-10 or IL-17, but not both).

The frequency of incidence of regulatory FoxP3⁺ CD4⁺ T cells in vivo in the tumor model mice versus the FVB mice was also investigated. A significant increase in FoxP3⁺CD4⁺ T cells was observed in the tumor-draining axillary and brachial lymph nodes of PyMT^{tg} mice as compared to FVB controls (8.6±0.9% versus 6.3±1.2%, p=0.033) (FIG. 11C). A significant increase in FoxP3⁺ CD4⁺ T cells was observed in the spleen of PyMT^{tg} mice as compared to FVB controls (12.3±5.4% versus 7.6±0.5%, p=0.027) (FIG. 11C). Very high frequencies of FoxP3⁺ CD4⁺ T_{reg} cells were observed in the tumors of PyMT^{tg} mice (19.0±7.8%) (FIG. 11C).

### Example 6

### TAM Display Analogies to Adipose Tissue Macrophages

It has recently been reported that F4/80⁺ adipose tissue macrophages (ATM) can in some cases acquire CD11c expression (Lumeng et al., J Clin Invest 117: 175-84, 2007), much as the above studies demonstrate that TAM do. Since certain diseases such as diabetes are associated with mild but chronic inflammation (Neels and Olefsky, J Clin Invest 116: 33-5, 2006), a comparison of the TAM and ATM cell populations was undertaken to better understand if ATM might contribute to this chronic inflammation much as the above results suggest that TAM contribute to tumor biology.

Diet-induced obese C57BI/6 male mice (Jackson Laboratory) were rendered insulin resistant by feeding them for 20 weeks with a high fat diet (HFD) consisting of 60 kcal % fat starting at 6 weeks of age. Db/db mice as well as young or age-matched control mice (fed a standard diet consisting of 10 kcal % fat) were also obtained. RBC-lysed single cell suspensions from axillary and brachial tumor draining and inguinal fat draining lymph nodes were used for FACS analysis. Briefly, naïve CD4⁺ T cells were prepared from RBC-lysed single cell suspensions from spleen, peripheral and mesenteric lymph nodes of FVB or C57BI/6 control mice. Cells were first MACS depleted of CD25⁺, CD69⁺ and CD103⁺ cells and then enriched with CD4-Microbeads (Miltenyi Biotech). Finally, CD62L⁺CD45Rb^{high}CD25⁻CD69⁻CD103⁻ naïve CD4⁺ T cells were isolated by FACS sorting. FACS and RT-PCR experiments were performed as described in the previous examples. Microscopy studies were performed on freshly isolated ATM, TAM and peritoneal macrophages collected from tissue samples by centrifugation and stained with hematoxylin and eosin stain using standard techniques.

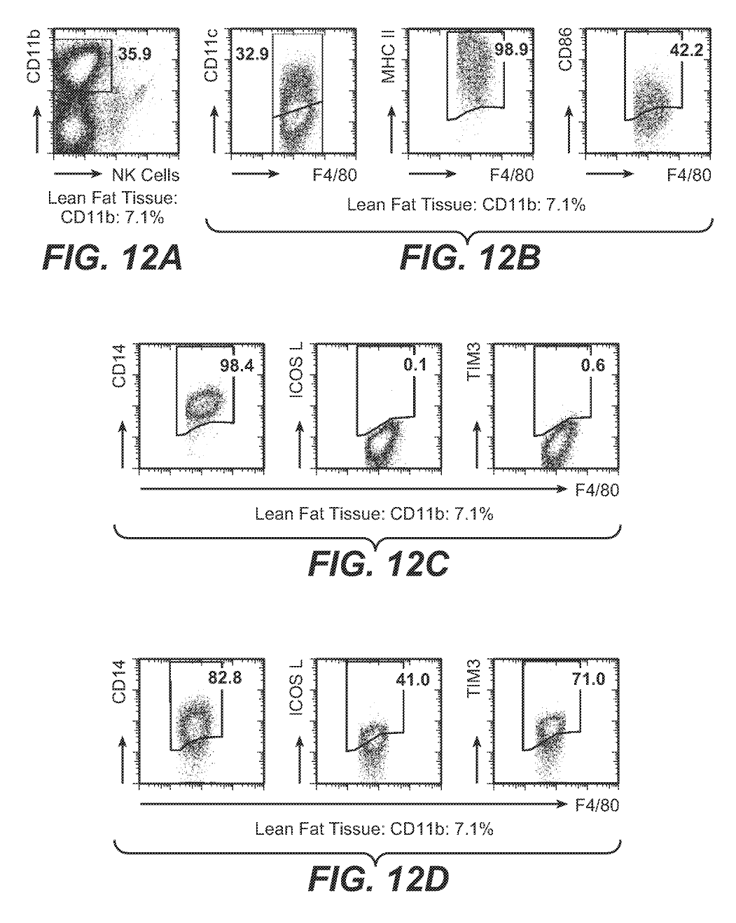

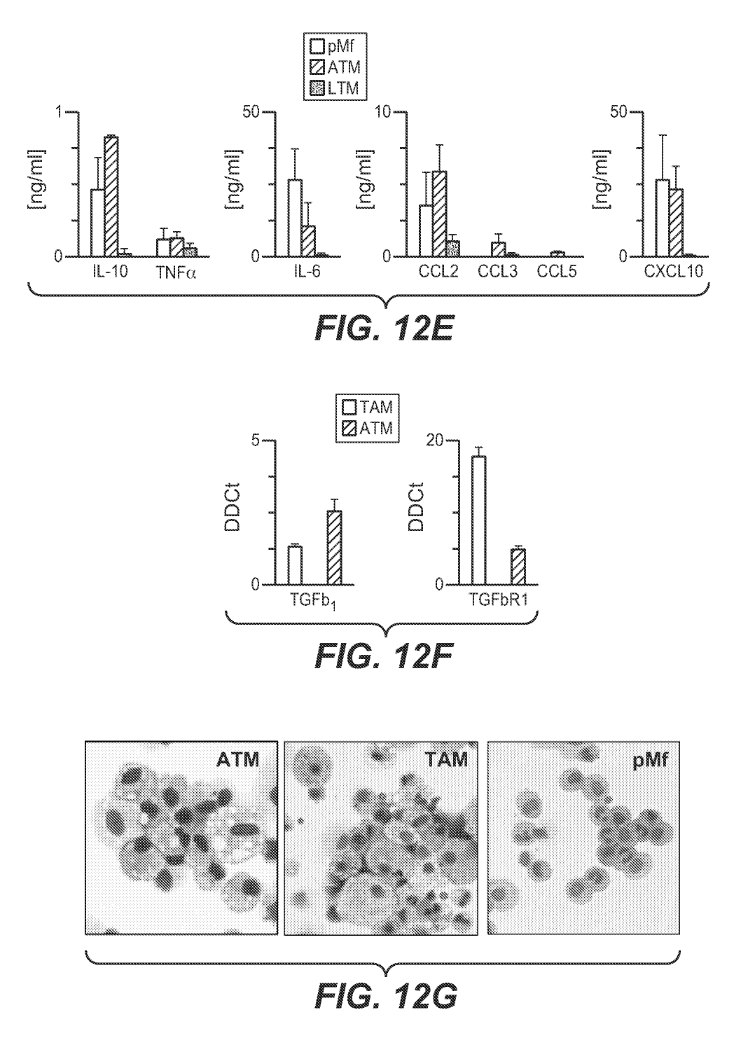

The results are shown in FIG. 12. Male C57BI/6 mice fed a high fat diet for 5 months displayed a high ratio of myeloid cells in their epididymal fat tissue, as did age-matched control mice (HFD: 35.9±6.7% (FIG. 12A); age-matched control mice: 32.9±7.1% (data not shown)). Two month old male C57BI/6 mice, however, displayed significantly lower ratios of CD11b⁺ cells in the epididymal fat tissue (15.5±11.0%, n=4 (data not shown)). Verifying earlier findings (Lumeng et al., J Clin Invest 117: 175-84, 2007), it was found that 32.9±6.7% of the F4/80⁺ ATM in the fat tissue also co-expressed CD11c (FIG. 12B), whereas macrophages isolated from epididymal fat tissue of age-matched or two-month-old mice fed a normal diet showed very little CD11c expression (data not shown). Further, ATM were found to also express high levels of MHC II and low levels of CD86, similar to the findings for TAM, above (FIG. 12B). Additional TAM surface markers identified in the studies above were also examined in the ATM population. It was found that ATM and TAM have similar expression levels of CD14, but ATM lack expression of ICOS L and TIM3, both of which show moderate to strong expression on TAM (compare FIGS. 12C and 12D).

Notably, the cytokine and chemokine profile of ATM purified from male C57BI/6 mice fed a high fat diet for 20 weeks was similar to that of TAM. These particular ATM expressed high levels of IL-10 (0.84±0.01 ng/ml vs. 0.47±0.18 ng/ml observed in peritoneal macrophages), intermediate levels of IL-6 (10.9±7.9 ng/ml vs. 38.1±30.6 ng/ml observed in peritoneal macrophages), and low levels of TNFα (0.13±0.04 ng/ml vs. 0.13±0.06 ng/ml in peritoneal macrophages) (FIG. 12E). ATM were also found to secrete high levels of CCL2 (5.9±1.8 ng/ml vs. 4.3±2.7 ng/ml observed in peritoneal macrophages) and CXCL10 (23.5±8.1 ng/ml vs. 28.7±16.5 ng/ml observed in peritoneal macrophages), but low levels of CCL3 (0.97±0.57 ng/ml vs. undetectable amounts in peritoneal macrophages) and CCL5 (0.2±0.2 ng/ml versus not detectable in peritoneal macrophages) (see FIG. 12E). Furthermore, ATM expressed similar levels of TGFβ₁ and slightly lower levels of TGFβR1 (3.6-fold less compared to TAM, but 4.9-fold more than peritoneal macrophages) (FIG. 12F). However, in sharp contrast to TAM, ATM did not express Runx3 or IRF-8 (data not shown), which correlates with the lack of langerin expression in ATM. Microscopy studies further suggested that the morphologies of ATM and TAM were similar to one another, but distinct from peritoneal macrophage morphology (FIG. 12G). TAM and ATM were both large in size with small nuclei and large vacuolated cytoplasms (see FIG. 12G).

The results indicated that F4/80⁺CD11c⁺ macrophages were not distinct immune cell subsets restricted to special microenvironments, but rather characterize a novel subpopulation of macrophages present in inflamed tissue. Further, this macrophage subpopulation itself consists of at least two subtypes having different cytokine expression and cell surface marker expression.

### Example 7

### ATM Effect on CD4⁺ T Cells

The Examples above demonstrated that TAM are able to induce FoxP3⁺ regulatory T cells (see FIG. 10A). Similar experiments were undertaken to determine whether FoxP3⁺ CD4⁺ T cells were increased in representation in obese high fat diet (HFD)-fed mice, and also whether ATM are similarly able to induce FoxP3⁺ regulatory T cells.

Naïve FoxP3⁻ CD4⁺ T cells were activated with the respective tissue and anti-CD3. 1×10⁴ adipose tissue macrophages (ATM) from obese mice were plated in round-bottom 96-well plates with 0.002 μg/mL anti-CD3 (BD Bioscience) and 5×10⁴ naïve CD4⁺ T cells and cultured at a final volume of 200 μL (complete RPMI1640 at 37° C., 5% CO₂). After five days CD4⁺ T cells were harvested and analyzed for FoxP3 expression.

To determine whether ATM induce FoxP3⁺ regulatory T cells, 2×10⁴ CD11c⁺ ATM, peritoneal macrophages or lean fat tissue macrophages (LTM) were cultured with 1×10⁵ naïve FoxP3⁺ CD4⁺ T cells and 0.002 μg/ml anti-CD3 for five days. Cells were either subsequently fixed and stained for Fox P3, or culture supernatants were harvested and tested for the presence of IL-2, IL-4, IL-10, and IL-17. Heatmap analyses were performed as described in Example 3. To confirm the differential expression of CCL2, CCL3, CCL5 and CXCL10 peritoneal macrophages from wildtype FVB mice or PyMT^{tg}-derived TAM were cultured at a concentration of 2×10⁶/ml without further stimulation. Chemokines secreted into the supernatant were analyzed after 21 hours. Real-time RT-PCR was performed as described in Example 3. Interleukin measurements were performed as described in Example 4.

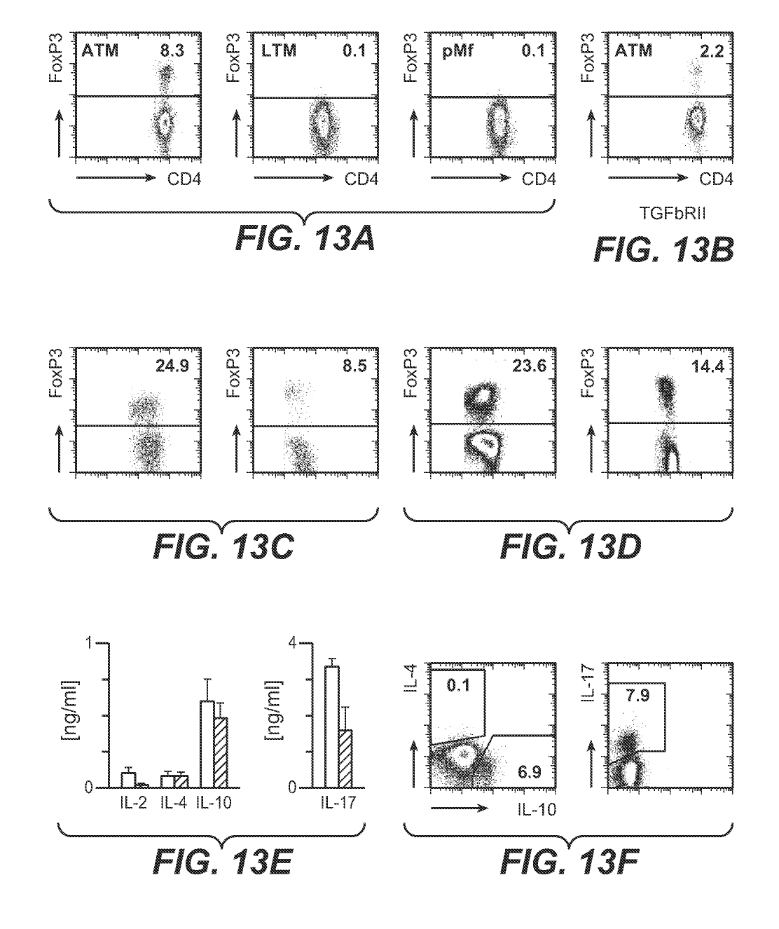

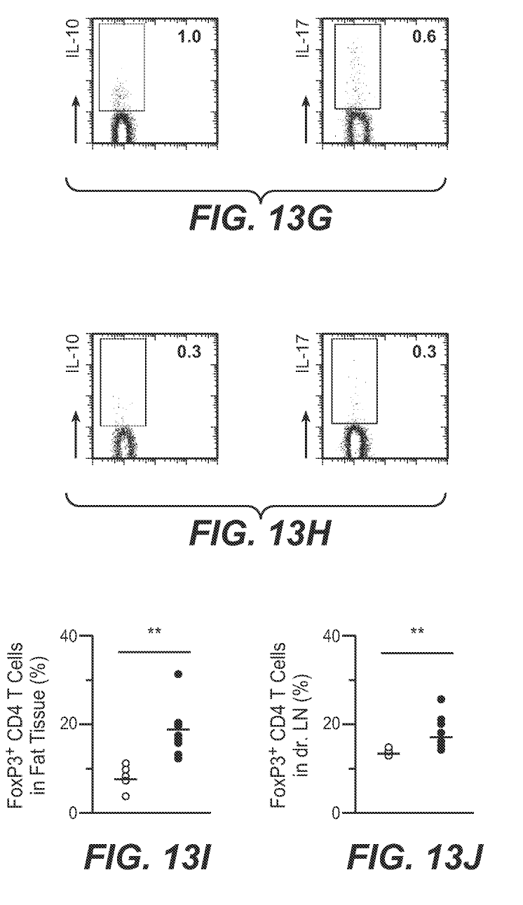

Similar to the immune infiltrate in tumors, the epidiymal fat tissue from obese HFD mice contained a significantly higher percentage of FoxP3⁺CD4⁺ T cells as compared to age-matched controls (18.5±6.2% vs. 7.9±3.7% in controls; see FIG. 13I). Furthermore, fat-draining lymph nodes from obese HFD mice also contained significantly higher levels of FoxP3⁺CD4⁺ T cells as compared to age-matched controls (17.2±3.3% vs. 13.4±0.6% in controls; see FIG. 13J).

As shown in FIG. 13A, CD11c⁺ ATM, but not peritoneal macrophages or lean fat tissue macrophages were able to induce FoxP3⁺ regulatory T cells (8.3±1.7% of the activated naïve T cells; compare left panel to center and right panels). This in vitro data was further supported by in vivo data. As observed in tumor bearing mice, increased levels of FoxP3⁺ T regulatory cells were detected among splenic (23.6±1.6% vs. 14.4±1.6% in controls) as well as epididymal fat tissue (24.9±6.2% vs. 8.5±1.3% in controls) CD4⁺ T cells in obese Db/Db mice (FIGS. 13C and 13D), tissues were ATM are known to be increased in prevalence. Since ATM are known to express TGFβ₁, a cytokine that has previously been shown to be important for the differentiation of regulatory T cells, the impact of this cytokine on ATM induction of FoxP3⁺ cells was assessed. Blockade of TGFβ by incorporation of TGFβRII-Fc into the assay almost completely repressed the induction of FoxP3⁺ T cells by CD11c⁺ ATM (2.2±0.1% of the activated naïve T cells) (FIG. 13B).

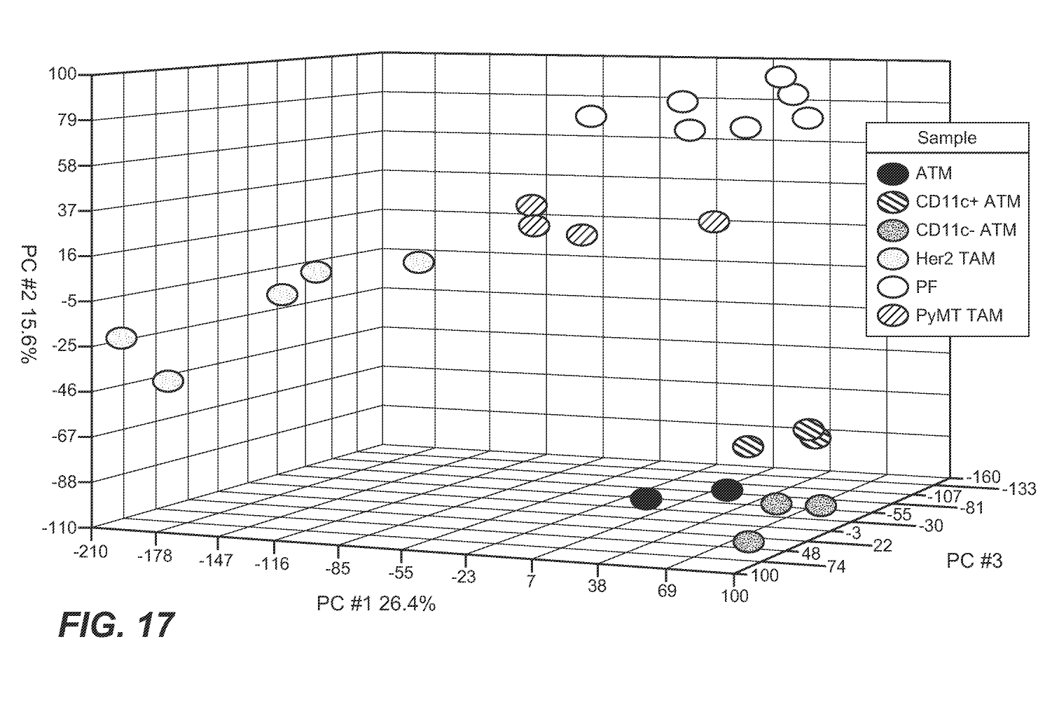

The ability of CD11c⁺ ATM to induce other types of T cells was also assessed. As shown in FIG. 13E, CD11c⁺ ATM activated naïve T cells not only included a population of FoxP3⁺ regulatory T cells, but they also displayed a Trl and TH₁₇ cytokine profile. Specifically, CD11c⁺ ATM activated naïve T cells secreted high levels of IL-10 (0.5±0.1 ng/ml vs. 0.6±0.15 ng/ml in T cells activated with peritoneal macrophages) and very low levels of IL-2)(0.02±0.01 ng/ml versus 0.1±0.04 ng/ml in T cells activated with peritoneal macrophages) and IL-4 (0.1±0.03 ng/ml versus 0.1±0.03 ng/ml in T cells activated with peritoneal macrophages). Further, ATM induced high levels of IL-17 expressed in T cells (1.6±0.6 ng/ml versus 3.4±0.2 ng/ml in T cells activated with peritoneal macrophages). FACS analyses of ATM-induced T cell culture samples confirmed that ATM stimulated the induction of Trl and TH₁₇ T cells from naïve T cell cultures (FIG. 13F). In addition, naïve T cells stimulated with CD11c⁺ ATM secreted significant amounts of TNFα, IL-5, and IL-13 (FIG. 16B. In analogy to the results of the above experiments on PyMT^{tg} mice, increased levels of IL-10 (1.0±0.2% vs. 0.3±0.1% in controls) and IL-17 (0.6±0.2% vs. 0.3±0.1% in controls) producing cells were detected in the fat draining lymph nodes of obese mice fed a high fat diet compared to control mice (FIGS. 13G and 13H). Although CD11c⁺ ATM and CD11c⁺ TAM seemed to behave similarly under inflammatory conditions, a PCA analysis revealed that ATM and TAM are distinct cellular populations among tissue macrophages (FIG. 17).

It was shown in the above examples that TAM do not fit into either the canonical M1 or M2 macrophage categories, despite literature reports to the contrary (see Example 3). The literature has suggested that ATM display an M1 phenotype (Lumeng et al., J Clin Invest 117: 175-84, 2007). In fact the results shown here suggest that ATM secrete IL-10, TGFα and IL-1 RA, as well as expressing Mgl1, Mgl2, CD14, and CD163—typical features of the M2 phenotype. These results also clearly show that ATM induce the production of suppressive regulatory T cells as well as inflammatory TH17 cells, and thus ATM span properties of both the M1 and M2 macrophage classes, much as TAM do.

### Example 8

### Functional Differences Between CD11c+ATM and CD11c⁻ ATM

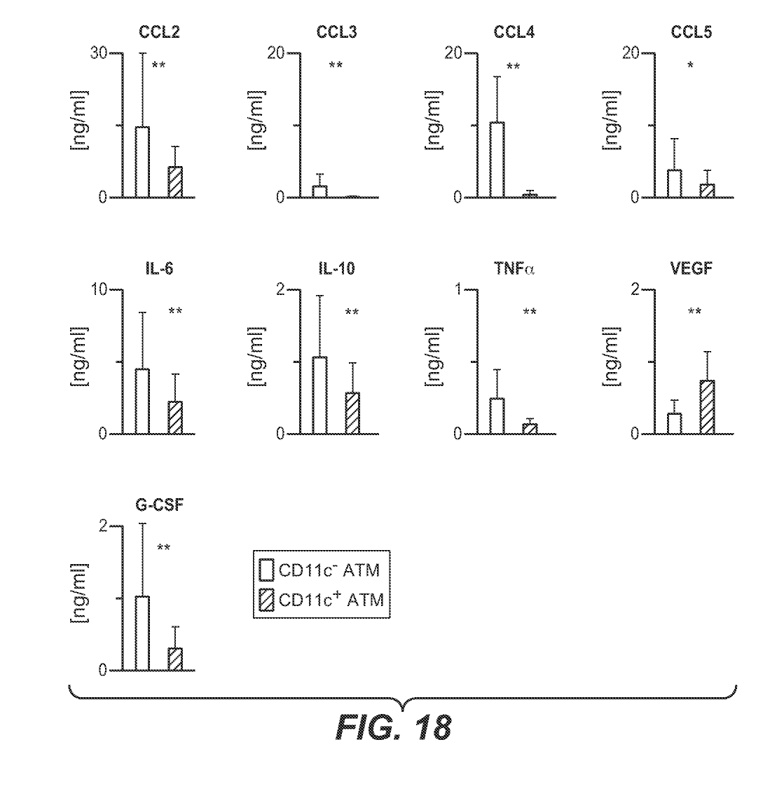

As shown in Example 3, TAM display characteristic cytokine expression profiles. The cytokine expression profiles of CD11c⁻ and CD11c⁺ ATM were examined. CD11c⁺ and CD11c⁻ ATM were purified from diet-induced obese C57BI/6 male mice as described above and the production of certain cytokine and chemokine proteins in each cell population was assessed after 21 hours of culture. FACS analysis was performed as described in Example 1. ATM were cultured in fibronectin-coated round-bottom 96 well-plates for 21 hours at a concentration of 2×10⁶/mL in RPMI1640 medium at 37° C. and 5% CO₂. Cytokines secreted in the supernatant were detected by Luminex analysis. The results are set forth in FIG. 18. CD11c⁻ ATM showed higher expression levels of CCL2, CCL3, CCL4, CCL5, IL-6, IL-10, TNFα, and G-CSF as compared to CD11c⁺ ATM. However, CD11c⁺ ATM showed higher expression levels of VEGF than CD11c⁻ ATM. M-CSF, IL-1b, MIG/CXCL9, MIP-2/CXCL2, RANTES, and KC/CXCL1 levels were similar between CD11c⁻ ATM and CD11c⁺ ATM, and neither CD11c⁻ ATM nor CD11c⁺ ATM expressed IL-1a or eotaxin (data not shown). This data further indicates that CD11⁻ ATM and CD11⁺ ATM are distinct cell populations likely to have different physiological functions, based on their distinct cytokine expression profiles.

As described in Example 4, TAM induce FoxP3⁺ T cells from naïve T cell populations. To investigate the T cell-priming potential of CD11c⁻ ATM and CD11c⁺ ATM, 1×10⁴ CD11c⁻ or CD11c⁺ ATM were cultured in round-bottom 96-well plates with 0.5 μg/mL anti-CD3 and 5×10⁴ naïve CD4⁺ T cells in a final volume of 200 μL. To foster the survival of ATM and naïve T cells, either the 96-well plates had previously been coated with recombinant murine fibronectin or recombinant human IL-2 was added to the cultures. After five days, the supernatants were harvested and stored at −80° C. prior to analysis. Cytokines and chemokines in the supernatants were detected later in thawed supernatants by cytokine ELISAs (Lincoplex™ kits (Linco), per the manufacturer's instructions). For assessment of cytokine production in the CD4⁺ T cells, single cell suspensions of draining lymph node or cultured cells were restimulated with PMA/ionomycin for six hours with the addition of Brefeldin A for the final four hours. Prior to staining, cells were blocked with appropriate sera or purified IgG. Acquisition included PI exclusion (surface stains) and was performed on a FACSCalibur or LSR II (Becton Dickinson) and analyzed with JoFlo software (Tree Star).

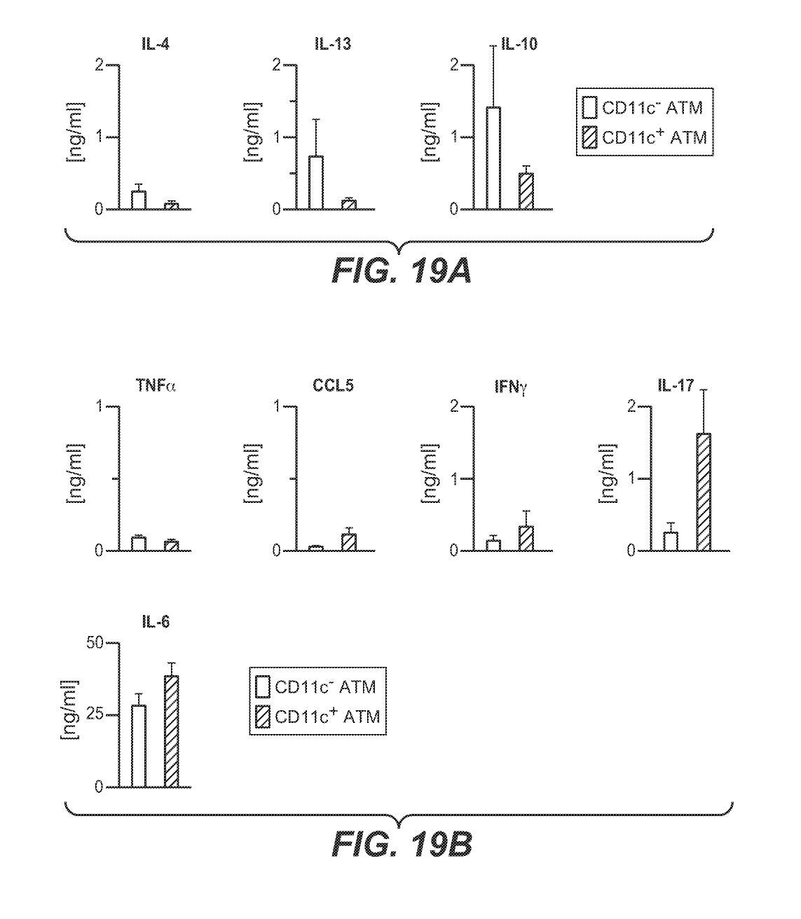

The results are depicted in FIGS. 19A and 19B. CD11c⁻ ATM induced T cells with slight increases in IL-4, TNFα, CCL5, IFNγ, and IL-17 expression, and much larger increases in IL-13, IL-10, and IL-5 expression (with IL-6 expression being over 25-fold increased) relative to unstimulated T cells. CD11c⁺ ATM induced T cells with slight to modest increases in IL-4, IL-13, IL 10, TNFα, CCL5, and IFNγ expression, and larger increases in expression of IL-17 and IL-6 (with IL-6 expression being over 30-fold increased) relative to unstimulated T cells. Comparing the cytokine/chemokine expression level patterns between the CD11c⁻ ATM-induced T cells and CD11c⁺ ATM-induced T cells demonstrates that CD11c⁻ ATM-induced cells display substantially greater expression of IL-10 and IL-13, similar expression of IL-4, TNFα, CCL5, and IFNγ, and substantially lesser expression of IL-17 and IL-6 than the CD11c⁺ ATM-induced cells (see FIGS. 19A and 19B). This result further demonstrates that CD11c⁻ ATM and CD11c⁺ ATM are two distinct cell populations with differing physiological functions.

### Example 9

### Induction of Th1 Versus Th2 Cells by ATM

One method by which dendritic cells activate CD4⁺ T cells to differentiate into Th1 or Th2 cells is by interaction of C-type lectin molecules on their surface with the naïve CD4⁺ T cells. Certain C-type lectins may bias induction of Th2 cells, for example, SIGN-R1 and DC-SIGN (Wieland et al., Microbes Infect. (2007) 9:134-41; Soilleux et al., J. Pathol. (2006) 209: 182-9; Bergman et al., J. Exp. Med. (2004) 200: 979-90; Ryan et al., J. Immunol. (2002) 169: 5638-48; and 't Hart and van Kooyk, Trends Immunol. (2004) 7: 353-359). Because ATM have characteristics of both macrophages and dendritic cells, the expression of certain of these C-type lectins by ATM was investigated. Briefly, microarray analysis of mRNA expression of DC-SIGN(CD209a), SIGN-R1 (CD209b), and SIGN-R2 (CD209c) in CD11c⁺ ATM and CD11c⁻ ATM cell populations was performed using microarray analyses as described in Example 2. FACS analysis of expression of SIGN-R1 protein in a mixed population of ATM cells was performed according to the protocol set forth in Example 1.

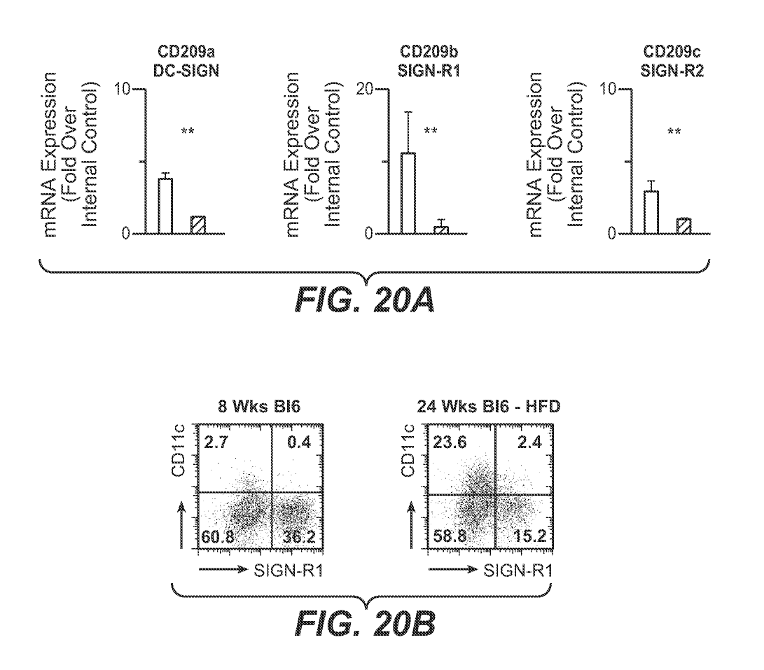

A microarray analysis of mRNA expression of various C-type lectins in CD11c⁻ ATM and CD11c⁺ ATM revealed that CD11c⁻ ATM express significantly greater amounts of mRNA for each of DC-SIGN, SIGN-R1 and SIGN-R2 than CD11c⁺ ATM do (see FIG. 20A). FACS studies of expressed protein showed that immune cells taken from the lymph nodes of normal 8-week-old BI6 mice fed a regular diet (i.e., nonobese mice) contained significant numbers of CD11⁻ cells expressing SIGN-R1 (FIG. 20B, left panel). However, samples from BI6 mice fed a high fat diet for 24 weeks (i.e., obese mice) contained a substantially greater number of CD11c⁺ cells, less than 10% of which expressed SIGN-R1 (FIG. 20B, right panel). Together, this data suggests that inflammatory ATM (CD11c⁺ DC-SIGN⁻) cell populations increase and anti-inflammatory ATM (CD11c⁻ DC-SIGN⁺) cell populations decrease in mice fed a HFD.

### Example 10

### T-Cell Priming by Different ATM Populations

The T-cell priming potential of CD11c⁻ and CD11c⁺ ATM are investigated by culturing 1×10⁴ CD11c⁻ and CD11c⁺ ATM in round-bottom 96-well plates with 0.5 μg/mL anti-CD3 and 5×10⁴ naïve CD4⁺ T cells in a final volume of 200 μL. To foster the survival of ATM and naïve T cells, the 96-well plate can be coated with recombinant murine fibronectin or recombinant human IL-2 can be added to the cultures. SIGN-R1 signaling is blocked by the addition of 10 μg/mL anti-SIGN-R1 or 10 μg/mL recombinant human ICAM-3. After five days of growth, the culture supernatants are harvested and stored at −80° C. prior to analysis. Cytokines and chemokines of the supernatants can be detected in thawed supernatants by cytokine ELISAs as described in the previous examples (i.e., using Lincoplex™ kits per the manufacturer's instructions).

Taken together, these experiments show that TAM display a phenotype of professional tolerogenic APC and can induce IL-10⁺ Trl, FoxP3⁺ T regulatory cells and TH17 T cells. TAM share certain phenotypic and functional analogies with ATM, suggesting that tissue macrophages acquire some similar characteristics as TAM (and yet retain some distinguishing features) under diverse inflammatory conditions. The commonalities between ATM and TAM may help to explain the observed correlation between obesity and carcinogenesis in mice and humans (Yakar et al., Endocrinology 147(12):5826-34, 2006; Calle et al., N Engl J Med 348(17): 1625-38, 2003), and the correlation of type 2 diabetes with 10-20% elevated risk of breast cancer (Wolf et al., Lancet Oncol. 6(2): 103-11, 2005). In both diseases a mild but chronic inflammation is accompanied by a prominent accumulation of macrophages in the affected tissues (Balkwill and Mantovani, Lancet, 357 (9255): 539-45, 2001; Neels and Olefsky, J Clin Invest 116: 33-5, 2006), and studies have linked increased numbers of tissue macrophages with chronic inflammation and either tumor progression (Mantovani et al., Immunol Today 13: 265-70, 1992; Pollard, Nat Rev Cancer 4: 71-8, 2004) or insulin resistance (Kamei et al., J Biol Chem 281: 26602-14, 2006; Weisberg et al., J Clin Invest 116(1): 115-24, 2006; Arkan et al., Nat Med 11: 191-8, 2005). Similarly, the presence of CD11c⁺F4/80⁺ myeloid DC have been described in experimental autoimmune encephalomyelitis (EAE), another chronic inflammation model (Ponomarev et al., J Neurosci Res 81: 374-89, 2005; Fischer and Reichmann, J Immunol 166: 2717-26, 2001; Miller et al, Ann N Y Acad Sci 1103: 179-91, 2007). Although the authors state that the indicated immune cells are dendritic cells, in view of the results from the above examples, they may be activated macrophages. Further, alveolar macrophages, a subset of resident tissue macrophages found in lung tissue having mild but persistent inflammation, were also found to express CD11c (Padilla et al., J Immunol 174(12): 8097-105, 2005; Fulton et al., Infect Immun 72(4): 2101-10, 2004). Although F4/80 expression has not yet been demonstrated on those alveolar macrophages, the alveolar microenvironment is rich in pro- and anti-inflammatory cytokines, which, in view of the results from the preceding experiments, may induce the differentiation of this special subset of tissue resident macrophages.

### PARTIAL LIST OF REFERENCES

Arkan, M. C., Hevener, A. L., Greten, F. R., Maeda, S., Li, Z. W., Long, J. M., Wynshaw-Boris, A., Poli, G., Olefsky, J., and Karin, M.: IKK-beta links inflammation to obesity-induced insulin resistance. Nat Med 11: 191-8, 2005
Bailey, S. L., Schreiner, B., McMahon, E. J., and Miller, S. D.: CNS myeloid DCs presenting endogenous myelin peptides ‘preferentially’ polarize CD4+ T(H)-17 cells in relapsing EAE. Nat Immunol 8: 172-80, 2007
Balkwill, F., Mantovani, A.: Inflammation and cancer: back to Virchow ?, Lancet 2001, 357(9255): 539-45
Bates, G. J., Fox, S. B., Han, C., Leek, R. D., Garcia, J. F., Harris, A. L., and Banham, A. H.:

Quantification of regulatory T cells enables the identification of high-risk breast cancer patients and those at risk of late relapse. J Clin Oncol 24: 5373-80, 2006
Bettelli, E., Carrier, Y., Gao, W., Kom, T., Strom, T. B., Oukka, M., Weiner, H. L., and Kuchroo, V. K.: Reciprocal developmental pathways for the generation of pathogenic effector TH17 and regulatory T cells. Nature 441: 235-8, 2006
Beyer, M. and Schultze, J. L.: Regulatory T cells in cancer. Blood 108: 804-11, 2006
Bingle, L., Brown, N. J., and Lewis, C. E.: The role of tumour-associated macrophages in tumour progression: implications for new anticancer therapies. J Pathol 196: 254-65, 2002
Borkowski, T. A., Letterio, J. J., Farr, A. G., and Udey, M. C.: A role for endogenous transforming growth factor beta 1 in Langerhans cell biology: the skin of transforming growth factor beta 1 null mice is devoid of epidermal Langerhans cells. J Exp Med 184: 2417-22, 1996
Calle, E. E., Rodriguez, C., Walker-Thurmond, K., Thun, M. J.: Overweight, obesity, and mortality from cancer in a prospectively studied cohort of U.S. adults, N Engl J Med 2003, 348(17): 1625-38
Chen, S., Luo, D., Streit, W. J., and Harrison, J. K.: TGF-beta1 upregulates CX3CR1 expression and inhibits fractalkine-stimulated signaling in rat microglia. J Neuroimmunol 133: 46-55, 2002
Chen, S., Tuttle, D. L., Oshier, J. T., Knot, H. J., Streit, W. J., Goodenow, M. M., and Harrison, J. K.: Transforming growth factor-beta1 increases CXCR4 expression, stromal-derived factor-1 alpha-stimulated signalling and human immunodeficiency virus-1 entry in human monocyte-derived macrophages. Immunology 114: 565-74, 2005
Chen, X., Baumel, M., Mannel, D. N., Howard, O. M., and Oppenheim, J. J.: Interaction of TNF with TNF receptor type 2 promotes expansion and function of mouse CD4+ CD25+T regulatory cells. J Immunol 179: 154-61, 2007
Cho, H. J., Shashkin, P., Gleissner, C. A., Dunson, D., Jain, N., Lee, J. K., Miller, Y., and Ley, K.: Induction of dendritic cell-like phenotype in macrophages during foam cell formation. Physiol Genomics 29: 149-60, 2007
Choudhury, R. P., Lee, J. M., Greaves, D. R.: Mechanisms of disease: macrophage-derived foam cells emerging as therapeutic targets in atherosclerosis. Nat Clin Pract Cardiovasc Med 2005, 2 (6): 309-15
Cua, D. J. and Kastelein, R. A.: TGF-beta, a ‘double agent’ in the immune pathology war. Nat Immunol 7: 557-9, 2006
Curiel, T. J., Coukos, G., Zou, L., Alvarez, X., Cheng, P., Mottram, P., Evdemon-Hogan, M., Conejo-Garcia, J. R., Zhang, L., Burow, M., Zhu, Y., Wei, S., Kryczek, I., Daniel, B., Gordon, A., Myers, L., Lackner, A., Disis, M. L., Knutson, K. L., Chen, L., and Zou, W.: Specific recruitment of regulatory T cells in ovarian carcinoma fosters immune privilege and predicts reduced survival. Nat Med 10: 942-9, 2004
Denning, T. L., Wang, Y. C., Patel, S. R., Williams, I. R., and Pulendran, B.: Lamina propria macrophages and dendritic cells differentially induce regulatory and interleukin 17-producing T cell responses. Nat Immunol 8: 1086-94, 2007
Fain, J. N., Tichansky, D. S., and Madan, A. K.: Transforming growth factor beta1 release by human adipose tissue is enhanced in obesity. Metabolism 54: 1546-51, 2005
Fainaru, O., Woolf, E., Lotem, J., Yarmus, M., Brenner, O., Goldenberg, D., Negreanu, V., Bernstein, Y., Levanon, D., Jung, S., and Groner, Y.: Runx3 regulates mouse TGF-beta-mediated dendritic cell function and its absence results in airway inflammation. Embo J 23: 969-79, 2004
Fischer, H. G. and Reichmann, G.: Brain dendritic cells and macrophages/microglia in central nervous system inflammation. J Immunol 166: 2717-26, 2001
Flegal, K. M., Graubard, B. I., Williamson, D. F., Gail, M. H.: Cause-specific excess deaths associated with underweight, overweight, and obesity, Jama 2007, 298(17): 2028-37
Fulton, S. A., Reba, S. M., Pai, R. K., Pennini, M., Torres, M. Harding, C. V., Boom, W. H.: Inhibition of major histocompatibility complex II expression and antigen processing in murine alveolar macrophages by Mycobacterium bovis BCG and the 19-kilodalton mycobacterial lipoprotein, Infect Immun 2004, 72(4): 2101-10
Gordon, S.: Alternative activation of macrophages, Nat Rev Immunol 2003, 3 (1): 23-35
Hardy, C. L. and Balducci, L.: Hemopoietic alterations of cancer. Am J Med Sci 290: 196-205, 1985
Huehn, J., Siegmund, K., Lehmann, J. C., Siewert, C., Haubold, U., Feuerer, M., Debes, G. F., Lauber, J., Frey, O., Przybylski, G. K., Niesner, U., de la Rosa, M., Schmidt, C. A., Brauer, R., Buer, J., Scheffold, A., and Hamann, A.: Developmental stage, phenotype, and migration distinguish naïve- and effector/memory-like CD4+ regulatory T cells. J Exp Med 199: 303-13, 2004
Hiratsuka, S., Nakamura, K., Iwai, S., Murakami, M., Itoh, T., Kijima, H., Shipley, J. M., Senior, R. M., and Shibuya, M.: MMP9 induction by vascular endothelial growth factor receptor-1 is involved in lung-specific metastasis. Cancer Cell 2: 289-300, 2002
Jaksits, S., Kriehuber, E., Charbonnier, A. S., Rappersberger, K., Stingl, G., and Maurer, D.: CD34+ cell-derived CD14+ precursor cells develop into Langerhans cells in a TGF-beta 1-dependent manner. J Immunol 163: 4869-77, 1999
Kamei, N., To be, K., Suzuki, R., Ohsugi, M., Watanabe, T., Kubota, N., Ohtsuka-Kowatari, N., Kumagai, K., Sakamoto, K., Kobayashi, M., Yamauchi, T., Ueki, K., Oishi, Y., Nishimura, S., Manabe, I., Hashimoto, H., Ohnishi, Y., Ogata, H., Tokuyama, K., Tsunoda, M., Ide, T., Murakami, K., Nagai, R., and Kadowaki, T.: Overexpression of monocyte chemoattractant protein-1 in adipose tissues causes macrophage recruitment and insulin resistance. J Biol Chem 281: 26602-14, 2006
Kaplan, D. H., Jenison, M. C., Saeland, S., Shlomchik, W. D., and Shlomchik, M. J.: Epidermal langerhans cell-deficient mice develop enhanced contact hypersensitivity. Immunity 23: 611-20, 2005
Karin, M.: Nuclear factor-kappaB in cancer development and progression. Nature 441: 431-6, 2006
Kelly, P. M., Davison, R. S., Bliss, E., and McGee, J. O.: Macrophages in human breast disease: a quantitative immunohistochemical study. Br J Cancer 57: 174-7, 1988
Kissenpfennig, A. and Malissen, B.: Langerhans cells—revisiting the paradigm using genetically engineered mice. Trends Immunol 27: 132-9, 2006
Lee, J. H., Yu, H. H., Wang, L. C., Yang, Y. H., Lin, Y. T., and Chiang, B. L.: The levels of CD4+ CD25+ regulatory T cells in pediatric patients with allergic rhinitis and bronchial asthma. Clin Exp Immunol 148: 53-63, 2007
Leek, R. D., Harris, A. L., and Lewis, C. E.: Cytokine networks in solid human tumors: regulation of angiogenesis. J Leukoc Biol 56: 423-35, 1994
Lehmann, J., Huehn, J., de la Rosa, M., Maszyna, F., Kretschmer, U., Krenn, V., Brunner, M., Scheffold, A., and Hamann, A.: Expression of the integrin alpha Ebeta 7 identifies unique subsets of CD25+ as well as CD25− regulatory T cells. Proc Natl Acad Sci U S A 99: 13031-6, 2002
Leong, P. P., Mohammad, R., Ibrahim, N., Ithnin, H., Abdullah, M., Davis, W. C., and Seow, H. F.: Phenotyping of lymphocytes expressing regulatory and effector markers in infiltrating ductal carcinoma of the breast. Immunol Lett 102: 229-36, 2006
Lewis, C. E. and Pollard, J. W.: Distinct role of macrophages in different tumor microenvironments. Cancer Res 66: 605-12, 2006
Lewis, J. S., Landers, R. J., Underwood, J. C., Harris, A. L., and Lewis, C. E.: Expression of vascular endothelial growth factor by macrophages is up-regulated in poorly vascularized areas of breast carcinomas. J Pathol 192: 150-8, 2000
Lewis, C. and Murdoch, C.: Macrophage responses to hypoxia: implications for tumor progression and anti-cancer therapies. Am J Pathol 167: 627-35, 2005
Liang, C. P., Han, S., Senokuchi, T., and Tall, A. R.: The macrophage at the crossroads of insulin resistance and atherosclerosis. Circ Res 100: 1546-55, 2007
Lin, E. Y., Jones, J. G., Li, P., Zhu, L., Whitney, K. D., Muller, W. J., and Pollard, J. W.: Progression to malignancy in the polyoma middle T oncoprotein mouse breast cancer model provides a reliable model for human diseases. Am J Pathol 163: 2113-26, 2003
Lin, E. Y., Nguyen, A. V., Russell, R. G., and Pollard, J. W.: Colony-stimulating factor 1 promotes progression of mammary tumors to malignancy. J Exp Med 193: 727-40, 2001
Lin, E. Y. and Pollard, J. W.: Role of infiltrated leucocytes in tumour growth and spread. Br J Cancer 90: 2053-8, 2004
Lin, E. Y., Li, J. F., Gnatovskiy, L., Deng, Y., Zhu, L., Grzesik, D. A., Qian, H., Xue, X. N., and Pollard, J. W.: Macrophages regulate the angiogenic switch in a mouse model of breast cancer. Cancer Res 66: 11238-46, 2006
Lin, W. W. and Karin, M.: A cytokine-mediated link between innate immunity, inflammation, and cancer. J Clin Invest 117: 1175-83, 2007
Liyanage, U. K., Moore, T. T., Joo, H. G., Tanaka, Y., Herrmann, V., Doherty, G., Drebin, J. A., Strasberg, S. M., Eberlein, T. J., Goedegebuure, P. S., and Linehan, D. C.: Prevalence of regulatory T cells is increased in peripheral blood and tumor microenvironment of patients with pancreas or breast adenocarcinoma. J Immunol 169: 2756-61, 2002
Luhn, K., Simmons, C. P., Moran, E., Dung, N. T., Chau, T. N., Quyen, N. T., Thao le, T. T., Van Ngoc, T., Dung, N. M., Wills, B., Farrar, J., McMichael, A. J., Dong, T., and Rowland-Jones, S.: Increased frequencies of CD4+ CD25(high) regulatory T cells in acute dengue infection. J Exp Med 204: 979-85, 2007
Lumeng, C. N., Bodzin, J. L., and Saltiel, A. R.: Obesity induces a phenotypic switch in adipose tissue macrophage polarization. J Clin Invest 117: 175-84, 2007
Mahnke, K., Schmitt, E., Bonifaz, L., Enk, A. H., and Jonuleit, H.: Immature, but not inactive: the tolerogenic function of immature dendritic cells. Immunol Cell Biol 80: 477-83, 2002
Mantovani, A.: Tumor-associated macrophages in neoplastic progression: a paradigm for the in vivo function of chemokines. Lab Invest 71: 5-16, 1994
Mantovani, A., Bottazzi, B., Colotta, F., Sozzani, S., and Ruco, L.: The origin and function of tumor-associated macrophages. Immunol Today 13: 265-70, 1992
Mantovani, A., Sozzani, S., Locati, M., Allavena, P., and Sica, A.: Macrophage polarization: tumor-associated macrophages as a paradigm for polarized M2 mononuclear phagocytes. Trends Immunol 23: 549-55, 2002
Mantovani, A., Sica, A., Sozzani, S., Allavena, P., Vecchi, A., Locati, M.: The chemokine system in diverse forms of macrophage activation and polarization, Trends Immunol 2004, 25 (12): 677-86
Mantovani, A., Schioppa, T., Porta, C., Allavena, P., Sica, A.: Role of tumor-associated macrophages in tumor progression and invasion. Cancer Metastasis Rev 23: 315-22, 2006
Marshall, N. A., Christie, L. E., Munro, L. R., Culligan, D. J., Johnston, P. W., Barker, R. N., and Vickers, M. A.: Immunosuppressive regulatory T cells are abundant in the reactive lymphocytes of Hodgkin lymphoma. Blood 103: 1755-62, 2004
McHugh, R. S., Whitters, M. J., Piccirillo, C. A., Young, D. A., Shevach, E. M., Collins, M., Byrne, M. C.: CD4(+)CD25(+) immunoregulatory T cells: gene expression analysis reveals a functional role for the glucocorticoid-induced TNF receptor. Immunity 16: 311-23, 2002
Miller, S. D., McMahon, E. J., Schreiner, B., and Bailey, S. L.: Antigen presentation in the CNS by myeloid dendritic cells drives progression of relapsing experimental autoimmune encephalomyelitis. Ann N Y Acad Sci 1103: 179-91, 2007
Murdoch, C., Giannoudis, A., Lewis, C. E.: Mechanisms regulating the recruitment of macrophages into hypoxic areas of tumors and other ischemic tissues. Blood 104(8): 2224-34, 2001
Murdoch, C., Giannoudis, A., and Lewis, C. E.: Mechanisms regulating the recruitment of macrophages into hypoxic areas of tumors and other ischemic tissues. Blood 104: 2224-34, 2004
Neels, J. G. and Olefsky, J. M.: Inflamed fat: what starts the fire? J Clin Invest 116: 33-5, 2006
Numasaki, M., Watanabe, M., Suzuki, T., Takahashi, H., Nakamura, A., McAllister, F., Hishinuma, T., Goto, J., Lotze, M. T., Kolls, J. K., and Sasaki, H.: IL-17 enhances the net angiogenic activity and in vivo growth of human non-small cell lung cancer in SCID mice through promoting CXCR-2-dependent angiogenesis. J Immunol 175: 6177-89, 2005
O'Sullivan, C. and Lewis, C. E.: Tumour-associated leucocytes: friends or foes in breast carcinoma. J Pathol 172: 229-35, 1994
Padilla, J., Daley, E., Chow, A. Robinson, K., Parthasarathi, K. McKenzie, A. N., Tschernig, T., Kurup, V. P. Donaldson, D. D., Grunig, G.: IL-13 regulates the immune response to inhaled antigens, J Immunol 2005, 174(12): 8097-105
Pollard, J. W.: Tumour-educated macrophages promote tumour progression and metastasis. Nat Rev Cancer 4: 71-8, 2004
Ponomarev, E. D., Shriver, L. P., Maresz, K., and Dittel, B. N.: Microglial cell activation and proliferation precedes the onset of CNS autoimmunity. J Neurosci Res 81: 374-89, 2005
Rad, R., Brenner, L., Bauer, S., Schwendy, S., Layland, L., da Costa, C. P., Reindl, W., Dossumbekova, A., Friedrich, M., Saur, D., Wagner, H., Schmid, R. M., and Prinz, C.: CD25+/Foxp3+ T cells regulate gastric inflammation and Helicobacter pylori colonization in vivo. Gastroenterology 131: 525-37, 2006
Raes, G., Brys, L., Dahal, B. K., Brandt, J., Grooten, J., Brombacher, F., Vanham, G., Noel, W., Bogaert, P., Boonefaes, T., Kindt, A., Van den Bergh, R., Leenen, P. J., De Baetselier, P., and Ghassabeh, G. H.: Macrophage galactose-type C-type lectins as novel markers for alternatively activated macrophages elicited by parasitic infections and allergic airway inflammation. J Leukoc Biol 77: 321-7, 2005
Raes, G., De Baetselier, P., Noel, W., Beschin, A., Brombacher, F., and Hassanzadeh Gh, G.: Differential expression of FIZZ1 and Ym1 in alternatively versus classically activated macrophages. J Leukoc Biol 71: 597-602, 2002
Roncarolo, M. G., Gregori, S., Battaglia, M., Bacchetta, R., Fleischhauer, K., and Levings, M. K.: Interleukin-10-secreting type 1 regulatory T cells in rodents and humans. Immunol Rev 212: 28-50, 2006
Sadhu, C., Ting, H. J., Lipsky, B., Hensley, K., Garcia-Martinez, L. F., Simon, S. I., and Staunton, D. E.: CD11c/CD18: novel ligands and a role in delayed-type hypersensitivity. J Leukoc Biol 81: 1395-403, 2007
Serafini, P., De Santo, C., Marigo, I., Cingarlini, S., Dolcetti, L., Gallina, G., Zanovello, P., and Bronte, V.: Derangement of immune responses by myeloid suppressor cells. Cancer Immunol Immunother 53: 64-72, 2004
Schiavoni, G., Mattei, F., Borghi, P., Sestili, P., Venditti, M., Morse, H. C., 3rd, Belardelli, F., and Gabriele, L.: ICSBP is critically involved in the normal development and trafficking of Langerhans cells and dermal dendritic cells. Blood 103: 2221-8, 2004
Seo, N., Hayakawa, S., Takigawa, M., and Tokura, Y.: Interleukin-10 expressed at early tumour sites induces subsequent generation of CD4(+) T-regulatory cells and systemic collapse of antitumour immunity. Immunology 103: 449-57, 2001
Serafini, P., De Santo, C., Marigo, I., Cingarlini, S., Dolcetti, L., Gallina, G., Zanovello, P., and Bronte, V.: Derangement of immune responses by myeloid suppressor cells. Cancer Immunol Immunother 53: 64-72, 2004
Sica, A., Schioppa, T., Mantovani, A., and Allavena, P.: Tumour-associated macrophages are a distinct M2 polarised population promoting tumour progression: potential targets of anti-cancer therapy. Eur J Cancer 42: 717-27, 2006
Steinman, R. M., Hawiger, D., and Nussenzweig, M. C.: Tolerogenic dendritic cells. Annu Rev Immunol 21: 685-711, 2003
Takahashi, H., Numasaki, M., Lotze, M. T., and Sasaki, H.: Interleukin-17 enhances bFGF-, HGF- and VEGF-induced growth of vascular endothelial cells. Immunol Lett 98: 189-93, 2005
Tsunemi, S., Iwasaki, T., Imado, T., Higasa, S., Kakishita, E., Shirasaka, T., and Sano, H.: Relationship of CD4+ CD25+ regulatory T cells to immune status in HIV-infected patients. Aids 19: 879-86, 2005
Valladeau, J., Ravel, O., Dezutter-Dambuyant, C., Moore, K., Kleijmeer, M., Liu, Y., Duvert-Frances, V., Vincent, C., Schmitt, D., Davoust, J., Caux, C., Lebecque, S., and Saeland, S.: Langerin, a novel C-type lectin specific to Langerhans cells, is an endocytic receptor that induces the formation of Birbeck granules. Immunity 12: 71-81, 2000
Wan, Y. Y. and Flavell, R. A.: The roles for cytokines in the generation and maintenance of regulatory T cells. Immunol Rev 212: 114-30, 2006
Weaver, C. T., Harrington, L. E., Mangan, P. R., Gavrieli, M., and Murphy, K. M.: Th17: an effector CD4 T cell lineage with regulatory T cell ties. Immunity 24: 677-88, 2006
Weisberg, S. P., McCann, D., Desai, M., Rosenbaum, M., Leibel, R. L., and Ferrante, A. W., Jr.: Obesity is associated with macrophage accumulation in adipose tissue. J Clin Invest 112: 1796-808, 2003
Weisberg, S. P., Hunter, D., Huber, R., Lemieux, J., Slaymaker, S., Vaddi, K., Charo, I., Leibel, R. L., Ferrante, A. W., Jr.,: CCR2 modulates inflammatory and metabolic effects of high-fat feeding, J Clin Invest 2006, 116(1): 115-24
Wolf, I., Sadetzki, S., Catane, R., Karasik, A., Kaufman, B.: Diabetes mellitus and breast cancer 2005, 6(2): 103-11
Woolf, E., Brenner, O., Goldenberg, D., Levanon, D., and Groner, Y.: Runx3 regulates dendritic epidermal T cell development. Dev Biol 303: 703-14, 2007
Wyckoff, J. B., Wang, Y., Lin, E. Y., Li, J. F., Goswami, S., Stanley, E. R., Segall, J. E., Pollard, J. W., and Condeelis, J.: Direct visualization of macrophage-assisted tumor cell intravasation in mammary tumors. Cancer Res 67: 2649-56, 2007
Xu, D., Fu, J., Jin, L., Zhang, H., Zhou, C., Zou, Z., Zhao, J. M., Zhang, B., Shi, M., Ding, X., Tang, Z., Fu, Y. X., and Wang, F. S.: Circulating and liver resident CD4+ CD25+ regulatory T cells actively influence the antiviral immune response and disease progression in patients with hepatitis B. J Immunol 177: 739-47, 2006
Yakar, S., Nunez, N. P., Pennisi, P., Brodt, P., Sun, H., Fallavollita, L., Zhao, H., Scavo, L., Novosyadlyy, R., Kurshan, N., Stannard, B., East-Palmer, J., Smith, N. C., Perkins, S. N., Fuchs-Young, R., Barrett, J. C., Hursting, S. D., LeRoith, D.: Increased tumor growth in mice with diet-induced obesity: impact of ovarian hormones, Endocrinology 2006, 147(12):5826-34
Yamashiro, S., Takeya, M., Nishi, T., Kuratsu, J., Yoshimura, T., Ushio, Y., Takahashi, K.: Tumor-derived monocyte chemoattractant protein-1 induces intratumoral infiltration of monocyte-derived macrophage subpopulation in transplanted rat tumors. Am J Pathol 145: 856-67, 1994
Yang, D., Howard, O. M., Chen, Q., and Oppenheim, J. J.: Cutting edge: immature dendritic cells generated from monocytes in the presence of TGF-beta 1 express functional C—C chemokine receptor 6. J Immunol 163: 1737-41, 1999

We claim:

1. A method of identifying inflammation-related tissue macrophages (IRTM) within a cell sample, comprising contacting the cell sample with at least one first agent that specifically recognizes a cell surface marker specific for macrophages and at least one second agent that specifically recognizes a cell surface marker specific for dendritic cells and determining the presence of cells recognized by both the at least one first agent and the at least one second agent.

2. The method of claim 1, wherein the at least one first agent and/or the at least one second agent are antibodies or antigen-binding fragments thereof.

3. The method of claim 1, wherein the cell surface marker specific for macrophages is F4/80 and/or the cell surface marker specific for dendritic cells is CD11c.

4. The method of claim 1, wherein the IRTM is selected from a tumor-associated macrophage (TAM) and an adipose tissue macrophage (ATM).

5. A method of isolating TAM or ATM from a mixture of cells, comprising (a) contacting the cell sample with at least one first agent that specifically recognizes a cell surface marker specific for macrophages and at least one second agent that specifically recognizes a cell surface marker specific for dendritic cells, and (b) isolating cells recognized by both the at least one first agent and the at least one second agent.

6. The method of claim 5, wherein the at least one first agent and/or the at least one second agent are antibodies or antigen-binding fragments thereof.

7. The method of claim 5, wherein the cell surface marker specific for macrophages is F4/80 and/or the cell surface marker specific for dendritic cells is CD11c.

8. A method of diagnosing a proliferative disorder or staging a tumor in a subject, comprising determining the presence and/or activity of TAM in the subject.

9. The method of claim 8, wherein the determining step comprises contacting a sample of cells from the subject with at least one first agent that specifically recognizes a cell surface marker specific for macrophages and at least one second agent that specifically recognizes a cell surface marker specific for dendritic cells, and identifying cells recognized by both the at least one first agent and the at least one second agent.

10. The method of claim 9, wherein the at least one first agent and/or the at least one second agent are antibodies or antigen-binding fragments thereof.

11. The method of claim 9, wherein the cell surface marker specific for macrophages is F4/80 and/or the cell surface marker specific for dendritic cells is CD11c.

12. The method of claim 8, wherein the determining step comprises contacting a sample of cells from the subject with one or more agents that collectively specifically recognize two or more cell surface receptors expressed on TAM, and identifying cells recognized by the one or more agents.

13. A method of treating a tumor or inhibiting tolerogenesis in a subject, comprising modulating TAM viability or activity.

14. The method of claim 13, wherein modulating TAM viability or activity comprises at least one of selective removal of TAM from a tumor cell population or tumor sample, selectively killing TAM within a tumor cell population or tumor sample, and inhibiting TAM activity within a tumor cell population or tumor sample.

15. The method of claim 13, wherein inhibiting TAM activity comprises inhibiting secretion or activity of one or more TAM-secreted cytokine or TAM-secreted chemokine in the population or sample.

16. The method of claim 15, wherein inhibiting secretion or activity of one or more TAM-secreted cytokine or TAM-secreted chemokine comprises administering a TAM-secreted cytokine/chemokine binding agent and/or administering an antagonist of a TAM-secreted cytokine/chemokine.

17. The method of claim 16, wherein the TAM-secreted cytokine/chemokine binding agent is selected from an antibody or antigen-binding fragment, a receptor specific for the cytokine or chemokine, or a small molecule inhibitory to the activity of the cytokine/chemokine.

18. A method of treating an autoimmune disorder in a subject, comprising modulating TAM viability or activity.

19. The method of claim 18, wherein modulating TAM viability or activity comprises stimulating TAM activity.

20. The method of claim 19, wherein stimulating TAM activity comprises administering one or more compounds selected from the group consisting of a TAM agonist and an agonist of TAM-secreted cytokine/chemokine.

21. The method of claim 19, wherein stimulating TAM activity results in induction of at least one of FoxP3⁺ CD4⁺ T regulatory cells, IL-10⁺CD4⁺ T regulatory cells, and inflammatory TH₁₇ cells.

22. A method for selectively inducing growth and/or proliferation of at least one of FoxP3⁺ CD4⁺ T regulatory cells, IL-10⁺CD4⁺ Trl cells, and inflammatory TH₁₇ cells, comprising administering TAM to naïve T cells or otherwise exposing naïve T cells to TAM under conditions appropriate for normal cell growth.

23. The method of claim 22, further comprising administering one or more compounds selected from a TAM agonist and an agonist of TAM-secreted cytokine/chemokines.

24. The method of claim 22, further comprising isolating the induced FoxP3⁺ CD4⁺ T regulatory cells, IL-10⁺CD4⁺ Trl cells, and/or inflammatory TH₁₇ cells.

25. A method of treating an inflammatory disorder in a subject, comprising modulating IRTM viability or activity.

26. A method for selectively inducing growth and/or proliferation of FoxP3⁺ CD4⁺ T regulatory cells, IL-10⁺CD4⁺ Trl cells and/or inflammatory TH₁₇ cells comprising exposing naïve T cells to TAM and/or ATM under conditions appropriate for normal cell growth.

27. The method of claim 26, further comprising administering one or more compounds selected from a TAM agonist, an ATM agonist, an agonist of TAM-secreted cytokine/chemokines, and an agonist of ATM-secreted cytokine/chemokines.


SEQUENCE LISTING (2 sequences)

SEQ ID NO: 1 - protein, 4 aa, Artificial sequence
  sequence is synthesized
GSGS

SEQ ID NO: 2 - protein, 4 aa, Artificial sequence
  sequence is synthesized
GGGS